(19) United States
(12) Patent Application Publication
(10) Pub. No.: US 2010/0015141 A1
(43) Pub. Date: Jan. 21, 2010

(54) 4-PHENOXY-6-ARYL-1H-PYRAZOLO[3,4-D]PYRIMIDINE AND N-ARYL-6-ARYL-1H-PYRAZOLO[3,4-D]PYRIMIDIN-4-AMINE COMPOUNDS, THEIR USE AS MTOR KINASE AND PI3 KINASE INHIBITORS, AND THEIR SYNTHESES

(75) Inventors: Matthew Gregory Bursavich, Salt Lake City, UT (US); Pawel Wojciech Nowak, Woodcliff Lake, NJ (US); David Malwitz, Hopewell Junction, NY (US); Sabrina Lombardi, New Haven, CT (US); Adam Matthew Gilbert, Congers, NY (US); Nan Zhang, Bayside, NY (US); Semiramis Ayral-Kaloustian, Tarrytown, NY (US); James Thomas Anderson, Hillsdale, NJ (US); Natasja Brooijmans, New York, NY (US)

Correspondence Address: WYETH LLC;PATENT LAW GROUP, 5 GIRALDA FARMS, MADISON, NJ, 07940, US

(73) Assignee: Wyeth, Madison, NJ (US)

(21) Appl. No.: 12/506,291

(22) Filed: Jul. 21, 2009

Related U.S. Application Data
(60) Provisional application No. 61/082,371, filed on Jul. 21, 2008.

(51) Int. Cl. A61K 31/519; C07D 487/04; A61K 39/395; A61P 35/00

(52) U.S. Cl. 424/133.1; 544/262; 514/262.1

(57) ABSTRACT

The invention relates to 4,6-disubstituted-1H-pyrazolo[3,4-d]pyrimidin-4-amine compounds, including 4-phenoxy-6-aryl-1H-pyrazolo[3,4-d]pyrimidine and N-aryl-6-aryl-1H-pyrazolo[3,4-d]pyrimidin-4-amine compounds of the Formula I:

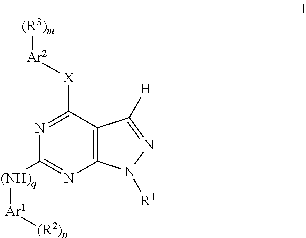

or a pharmaceutically acceptable salt thereof, wherein the constituent variables are as defined herein, compositions comprising the compounds, and methods for making and using the compounds.

### FIELD OF THE INVENTION

The invention relates to 4,6-disubstituted-1H-pyrazolo[3,4-d]pyrimidin-4-amine compounds, including 4-phenoxy-6-aryl-1H-pyrazolo[3,4-d]pyrimidine and N-aryl-6-aryl-1H-pyrazolo[3,4-d]pyrimidin-4-amine compounds, compositions comprising such a compound, methods of synthesizing such compounds, and methods for treating mTOR-related diseases comprising the administration of an effective amount of such a compound. The invention also relates to methods for treating PI3K-related diseases comprising the administration of an effective amount of such compounds including said 4-phenoxy-6-aryl-1H-pyrazolo[3,4-d]pyrimidine or N-aryl-6-aryl-1H-pyrazolo[3,4-d]pyrimidin-4-amine compounds.

### BACKGROUND OF THE INVENTION

Phosphatidylinositol (hereinafter abbreviated as “PI”) is one of the phospholipids in cell membranes. In recent years it has become clear that PI plays an important role also in intracellular signal transduction. It is well recognized in the art that PI (4,5) bisphosphate (PI(4,5)P2 or PIP2) is degraded into diacylglycerol and inositol (1,4,5) triphosphate by phospholipase C to induce activation of protein kinase C and intracellular calcium mobilization, respectively [M. J. Berridge et al., Nature, 312, 315 (1984); Y. Nishizuka, Science, 225, 1365 (1984)].

In the late 1980s, phosphatidylinositol-3 kinase (“PI3K”) was found to be an enzyme that phosphorylates the 3-position of the inositol ring of phosphatidylinositol [D. Whitman et al., Nature, 332, 664 (1988)]. When PI3K was discovered, it was originally considered to be a single enzyme. Recently however, it was clarified that a plurality of PI3K subtypes exists. Three major subtypes of PI3Ks have now been identified on the basis of their in vitro substrate specificity, and these three are designated class I (a & b), class II, and class III [B. Vanhaesebroeck, Trend in Biol. Sci., 22, 267(1997)].

The class Ia PI3K subtype has been most extensively investigated to date. Within the class Ia subtype there are three isoforms (α, β, & δ) that exist as hetero dimers of a catalytic 110-kDa subunit and regulatory subunits of 50-85 kDa. The regulatory subunits contain SH2 domains that bind to phosphorylated tyrosine residues within growth factor receptors or adaptor molecules and thereby localize PI3K to the inner cell membrane. At the inner cell membrane PI3K converts PIP2 to PIP3 (phosphatidylinositol-3,4,5-trisphosphate) that serves to localize the downstream effectors PDK1 and Akt to the inner cell membrane where Akt activation occurs. Activated Akt mediates a diverse array of effects including inhibition of apoptosis, cell cycle progression, response to insulin signaling, and cell proliferation. Class Ia PI3K subtypes also contain Ras binding domains (RBD) that allow association with activated Ras providing another mechanism for PI3K membrane localization. Activated, oncogenic forms of growth factor receptors, Ras, and even PI3K kinase have been shown to aberrantly elevate signaling in the PI3K/Akt/mTOR pathway resulting in cell transformation. As a central component of the PI3K/Akt/mTOR signaling pathway PI3K (particularly the class Ia α isoform) has become a major therapeutic target in cancer drug discovery.

Substrates for class I PI3Ks are PI, PI(4)P and PI(4,5)P2, with PI(4,5)P2 being the most favored. Class I PI3Ks are further divided into two groups, class Ia and class Ib, because of their activation mechanism and associated regulatory subunits. The class Ib PI3K is p110γ that is activated by interaction with G protein-coupled receptors. Interaction between p110γ and G protein-coupled receptors is mediated by regulatory subunits of 110, 87, and 84 kDa.

PI and PI(4)P are the known substrates for class II PI3Ks; PI(4,5)P2 is not a substrate for the enzymes of this class. Class II PI3Ks include PI3K C2α, C2β, and C2γ isoforms, which contain C2 domains at the C terminus, implying that their activity is regulated by calcium ions.

The substrate for class III PI3Ks is PI only. A mechanism for activation of the class III PI3Ks has not been clarified. Because each subtype has its own mechanism for regulating activity, it is likely that activation mechanism(s) depend on stimuli specific to each respective class of PI3K.

The compound PI103 (3-(4-(4-morpholinyl)pyrido[3′,2′:4,5]furo[3,2-d]pyrimidin-2-yl)phenol) inhibits PI3K_{α} and PI3K_{γ} as well as the mTOR complexes with IC₅₀ values of 2, 3, and 50-80 nM respectively. I.P. dosing in mice of this compound in human tumor xenograft models of cancer demonstrated activity against a number of human tumor models, including the glioblastoma (PTEN null U87MG), prostate (PC3), breast (MDA-MB-468 and MDA-MB-435) colon carcinoma (HCT 116); and ovarian carcinoma (SKOV3 and IGROV-1); (Raynaud et al, Pharmacologic Characterization of a Potent Inhibitor of Class I Phosphatidylinositide 3-Kinases, Cancer Res. 2007 67: 5840-5850).

The compound ZSTK474 (2-(2-difluoromethylbenzoimidazol-1-yl)-4,6-dimorpholino-1,3,5-triazine) inhibits PI3K_{α} and PI3K_{γ} but not the mTOR enzymes with IC₅₀ values of 16, 4.6 and >10,000 nM respectively (Dexin Kong and Takao Yamori, ZSTK474 is an ATP-competitive inhibitor of class I phosphatidylinositol 3 kinase isoforms, Cancer Science, 2007, 98:10 1638-1642). Chronic oral administration of ZSTK474 in mouse human xenograft cancer models, completely inhibited growth that originated from a non-small-cell lung cancer (A549), a prostate cancer (PC-3), and a colon cancer (WiDr) at a dose of 400 mg/kg. (Yaguchi et al, Antitumor Activity of ZSTK474, a New Phosphatidylinositol 3-Kinase Inhibitor, J. Natl. Cancer Inst. 98: 545-556).

The compound NVP-BEZ-235 (2-methyl-2-(4-(3-methyl-2-oxo-8-(quinolin-3-yl)-2,3-dihydro-1H-imidazo[4,5-c]quinolin-1-yl)phenyl)propanenitrile) inhibits both PI3K_{α} and PI3K_{γ} as well as the mTOR enzyme with IC₅₀ values 4, 5, and “nanomolar”. Testing in human tumor xenograft models of cancer demonstrated activity against human tumor models of prostrate (PC-3) and glioblastoma (U-87) cancer. It entered clinical trials in December of 2006 (Verheijen, J. C. and Zask, A., Phosphatidylinositol 3-kinase (PI3K) inhibitors as anticancer drugs, Drugs Fut. 2007, 32(6): 537-547).

The compound SF-1126 (a prodrug form of LY-294002, which is 2-(4-morpholinyl)-8-phenyl-4H-1-benzopyran-4-one) is “a pan-PI3K inhibitor”. It is active in preclinical mouse cancer models of prostrate, breast, ovarian, lung, multiple myeloma, and brain cancers. It began clinical trials in April, 2007 for the solid tumors endometrial, renal cell, breast, hormone refractory prostate, and ovarian cancers. (Verheijen, J. C. and Zask, A., Phosphatidylinositol 3-kinase (PI3K) inhibitors as anticancer drugs, Drugs Fut. 2007, 32(6): 537-547).

Exelixis Inc. (So. San Francisco, Calif.) recently filed INDs for XL-147 (a selective pan-PI3K inhibitor of unknown structure) and XL-765 (a mixed inhibitor of mTOR and PI3K of unknown structure) as anticancer agents. TargeGen's short-acting mixed inhibitor of PI3Kγ and δ, TG-100115, is in phase I/II trials for treatment of infarct following myocardial ischemia-reperfusion injury. Cerylid's antithrombotic PI3Kβ inhibitor CBL-1309 (structure unknown) has completed preclinical toxicology studies.

According to Verheijen, J. C. and Zask, A., Phosphatidylinositol 3-kinase (PI3K) inhibitors as anticancer drugs, Drugs Fut. 2007, 32(6): 537-547,
Although it seems clear that inhibition of the a isoform is essential for the antitumor activity of PI3K inhibitors, it is not clear whether a more selective inhibitor of a particular PI3K isoform may lead to fewer unwanted biological effects. It has recently been reported that non-PI3Kα class I isoforms (PI3Kβ, δ and γ) have the ability to induce oncogenic transformation of cells, suggesting that nonisoform-specific inhibitors may offer enhanced therapeutic potential over specific inhibitors.
Selectivity versus other related kinases is also an important consideration for the development of PI3K inhibitors. While selective inhibitors may be preferred in order to avoid unwanted side effects, there have been reports that inhibition of multiple targets in the PI3K/Akt pathway (e.g., PI3Kα and mTOR [mammalian target of rapamycin]) may lead to greater efficacy. It is possible that lipid kinase inhibitors may parallel protein kinase inhibitors in that nonselective inhibitors may also be brought forward to the clinic.

Mammalian Target of Rapamycin, mTOR, is a cell-signaling protein that regulates the response of tumor cells to nutrients and growth factors, as well as controlling tumor blood supply through effects on Vascular Endothelial Growth Factor, VEGF. Inhibitors of mTOR starve cancer cells and shrink tumors by inhibiting the effect of mTOR. All mTOR inhibitors bind to the mTOR kinase. This has at least two important effects. First, mTOR is a downstream mediator of the PI3K/Akt pathway. The PI3K/Akt pathway is thought to be over-activated in numerous cancers and may account for the widespread response from various cancers to mTOR inhibitors. The over-activation of the upstream pathway would normally cause mTOR kinase to be over-activated as well. However, in the presence of mTOR inhibitors, this process is blocked. The blocking effect prevents mTOR from signaling to downstream pathways that control cell growth. Over-activation of the PI3K/Akt kinase pathway is frequently associated with mutations in the PTEN gene, which is common in many cancers and may help predict what tumors will respond to mTOR inhibitors. The second major effect of mTOR inhibition is anti-angiogenesis, via the lowering of VEGF levels.

In lab tests, certain chemotherapy agents were found to be more effective in the presence of mTOR inhibitors. George, J. N., et al., Cancer Research, 61, 1527-1532, 2001. Additional lab results have shown that some rhabdomyosarcoma cells die in the presence of mTOR inhibitors. The complete functions of the mTOR kinase and the effects of mTOR inhibition are not completely understood.

There are three mTOR inhibitors, which have progressed into clinical trials. These compounds are Wyeth's Torisel, also known as 42-(3-hydroxy-2-(hydroxymethyl)-rapamycin 2-methylpropanoate, CCI-779 or Temsirolimus; Novartis' Everolimus, also known as 42-O-(2-hydroxyethyl)-rapamycin, or RAD 001; and Ariad's AP23573 also known as 42-(dimethylphopsinoyl)-rapamycin. The FDA has approved Torisel for the treatment of advanced renal cell carcinoma. In addition, Torisel is active in a NOS/SCID xenograft mouse model of acute lymphoblastic leukemia [Teachey et al, Blood, 107(3), 1149-1155, 2006]. On Mar. 30, 2009, the Food and Drug Administration (FDA) approved Everolimus (AFINITOR™) for the treatment of patients with advanced renal cell carcinoma. AP23573 has been given orphan drug and fast-track status by the FDA for treatment of soft-tissue and bone sarcomas.

The three mTOR inhibitors have non-linear, although reproducible pharmacokinetic profiles. Mean area under the curve (AUC) values for these drugs increase at a less than dose related way. The three compounds are all semi-synthetic derivatives of the natural macrolide antibiotic rapamycin. It would be desirable to find fully synthetic compounds, which inhibit mTOR that are more potent and exhibit improved pharmacokinetic behaviors.

As explained above, PI3K inhibitors and mTOR inhibitors are expected to be novel types of medicaments useful against cell proliferation disorders, especially as carcinostatic agents. Thus, it would be advantageous to have new PI3K inhibitors and mTOR inhibitors as potential treatment regimens for mTOR- and PI3K-related diseases. The instant invention is directed to these and other important ends.

### SUMMARY OF THE INVENTION

In one aspect, the invention provides compounds of the Formula I:

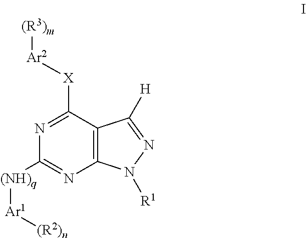

or a pharmaceutically acceptable salt thereof, wherein the constituent variables are as defined below.

In one aspect, the invention provides compounds of the Formula II:

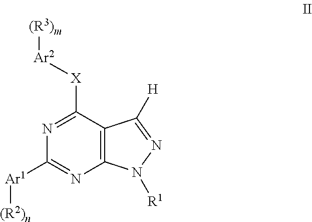

or a pharmaceutically acceptable salt thereof, wherein the constituent variables are as defined below.

In one aspect, the invention provides compounds of the Formula III:

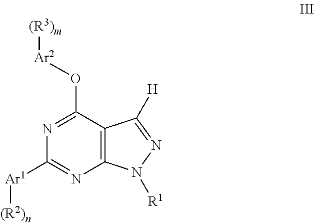

or a pharmaceutically acceptable salt thereof, wherein the constituent variables are as defined below.

In one aspect, the invention provides compounds of the Formula IV:

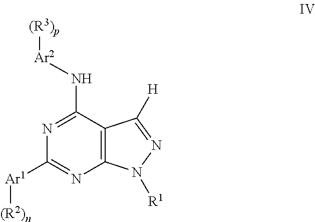

or a pharmaceutically acceptable salt thereof, wherein the constituent variables are as defined below.

In one aspect, the invention provides compounds of the Formula V:

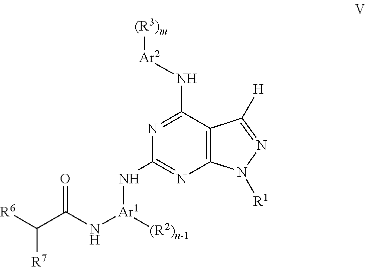

or a pharmaceutically acceptable salt thereof, wherein the constituent variables are as defined below.

In other aspects, the invention provides compositions comprising a compound of the invention, and methods for making compounds of the invention. In further aspects, the invention provides methods for inhibiting PI3K and mTOR in a subject, and methods for treating PI3K-related and mTOR-related disorders in a mammal in need thereof.

In other aspects, the invention provides further methods of synthesizing the compounds or pharmaceutically acceptable salts of compounds of the present Formulas I-V.

### DETAILED DESCRIPTION OF THE INVENTION

In one aspect, the invention provides compounds of the Formula I:

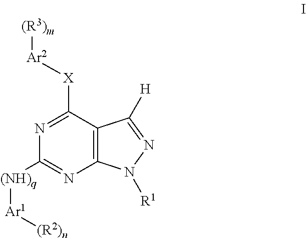

or a pharmaceutically acceptable salt thereof wherein;

q is 0 or 1 with the proviso that when q=1, then n is 1, 2, or 3 and at least one of R² is R⁶R⁷NC(O)NH—,

Ar¹ and Ar² are each independently phenyl, naphthyl, 1-oxo-2,3-dihydro-1H-isoindol-5-yl, or a nitrogen-containing mono- or bicyclic heteroaryl-;

X is —NH—, —N(C₁-C₆alkyl)-, —O—, or —S—;

R¹ is selected from: a) hydrogen; b) C₁-C₆alkyl- optionally substituted with from 1 to 3 substituents independently selected from: i) C₁-C₆alkoxy-, ii) (C₁-C₆alkyl)amino-, iii) di(C₁-C₆alkyl)amino-, iv) HC(O)—, v) HO₂C—, and vi) (C₁-C₆alkoxy)carbonyl-; c) C₁-C₆aminoalkyl- optionally substituted with from 1 to 3 substituents independently selected from: i) C₆-C₁₄aryl- optionally substituted with halogen, ii) (C₁-C₉heteroaryl)alkyl-, iii) (C₆-C₁₄aryl)alkyl-, iv) H₂N—C₁-C₆alkylene-, v) (C₁-C₆alkyl)amino-C₁-C₆alkylene-, and vi) di(C₁-C₆alkyl)amino-C₁-C₆alkylene-; d) carbonylamidoalkyl- optionally substituted with a substituent selected from: i) halogen, or ii) di(C₁-C₆alkyl)amino-; e) C₃-C₈cycloalkyl- optionally substituted with from 1 to 3 substituents independently selected from: i) C₁-C₆alkoxy-, ii) (C₁-C₆alkyl)amino-, iii) di(C₁-C₆alkyl)amino-, iv) HC(O)—, v) HO₂C—, and vi) (C₁-C₆alkoxy)carbonyl-; f) C₆-C₁₄aryl- optionally substituted with a substituent selected from: i) HO₂C—, ii) C₁-C₆hydroxylalkyl-, iii) R⁴R⁵NC(O)—, or iv) (C₁-C₆alkoxy)carbonyl-; g) C₁-C₉heterocycle- optionally substituted with from 1 to 3 substituents independently selected from: i) C₁-C₈acyl-, wherein the C₁-C₈acyl- is optionally substituted with a H₂N—, ii) C₁-C₆alkyl-, iii) (C₁-C₉heteroaryl)alkyl- wherein the ring portion of the (C₁-C₉heteroaryl)alkyl- group is optionally substituted with from 1 to 3 substituents independently selected from: (A) C₁-C₆alkylC(O)NH—, (B) halogen, (C) H₂N—, and (D) C₁-C₆alkyl-, iv) C₁-C₉heterocyclyl(C₁-C₆alkyl)-, wherein the ring portion of the C₁-C₉heterocyclyl(C₁-C₆alkyl)- group is optionally substituted by a (C₆-C₁₄aryl)alkyl-, v) (C₆-C₁₄aryl)alkyl-, wherein the ring portion of the (C₆-C₁₄aryl)alkyl- group is optionally substituted by 1 to 3 substituents independently selected from: (A) halogen, (B) C₁-C₆alkyl-, (C) di(C₁-C₆alkyl)amino-(C₁-C₆alkylene)-O—, and (D) and C₁-C₉heteroaryl-; and vi) (C₁-C₆alkoxy)carbonyl-; h) C₁-C₉heterocyclyl(C₁-C₆alkyl)- optionally substituted with a substituent selected from: i) C₁-C₆alkyl-, ii) C₃-C₈cycloalkyl-, iii) (C₁-C₆alkoxy)carbonyl-, iv) C₁-C₆alkylcarboxy-, v) (C₆-C₁₄aryl)alkyl- wherein the ring portion of the (C₆-C₁₄aryl)alkyl- group is optionally substituted with a substituent selected from: (A) halogen, (B) C₁-C₉heteroaryl-, or (C) di(C₁-C₆alkyl)amino-(C₁-C₆alkylene)-O—, vi) (C₁-C₉heteroaryl)alkyl- wherein the ring portion of the (C₁-C₉heteroaryl)alkyl- group is optionally substituted by a halogen, or vii) C₁-C₈acyl-, wherein the C₁-C₈acyl- is optionally substituted with from 1 to 3 independently selected halogens, i) (C₁-C₉heteroaryl)alkyl- wherein the ring portion of the (C₁-C₉heteroaryl)alkyl- is optionally substituted by 1 to 3 substituents independently selected from: i) R⁴R⁵NC(O)NH—, ii) (C₁-C₆alkoxy)carbonyl-, iii) HO₂C—, iv) hydroxyl, and v) R⁴R⁵NC(O)—; j) (C₆-C₁₄aryl)alkyl- wherein the ring portion of the (C₆-C₁₄aryl)alkyl- group is optionally by 1 to 3 substituents independently selected from: i) R⁴R⁵NC(O)NH—, ii) (C₁-C₆alkoxy)carbonyl-, iii) HO₂C—, iv) hydroxyl, v) and R⁴R⁵NC(O)—; k) C₁-C₆hydroxylalkyl-; l) C₁-C₆perfluoroalkyl-; or m) C₁-C₉heteroaryl- optionally substituted with a substituent selected from: i) HO₂C—, ii) C₁-C₆hydroxylalkyl-, iii) R⁴R⁵NC(O)—, or iv) (C₁-C₆alkoxy)carbonyl-;

R⁴ and R⁵ are each independently selected from: a) H; b) C₁-C₆alkyl- optionally substituted with a substituent selected from: i) C₁-C₆alkylC(O)NH—, ii) H₂N—, iii) (C₁-C₆alkyl)amino-, or iv) di(C₁-C₆alkyl)amino-; c) C₃-C₈cycloalkyl- optionally substituted with a substituent selected from: i) C₁-C₆alkylC(O)NH—, ii) H₂N—, iii) (C₁-C₆alkyl)amino-, or iv) or di(C₁-C₆alkyl)amino-; d) C₆-C₁₄aryl- optionally substituted with a substituent selected from: i) halogen, or ii) monocyclic C₁-C₆heterocycle- wherein the monocyclic C₁-C₆heterocycle- is optionally substituted with (C₁-C₆alkoxy)carbonyl-; e) C₁-C₉heteroaryl-; f) (C₁-C₉heteroaryl)alkyl-; g) C₁-C₉heterocyclyl(C₁-C₆alkyl)-; h) (C₆-C₁₄aryl)alkyl-, wherein the chain portion of the (C₆-C₁₄aryl)alkyl- group is optionally substituted by a hydroxyl; or i) monocyclic C₁-C₆heterocycle- optionally substituted with a (C₁-C₆alkoxy)carbonyl-;

or R⁴ and R⁵, when taken together with the nitrogen to which they are attached, form a 3- to 7-membered heterocycle- wherein up to two of the carbon atoms of the heterocycle- are optionally replaced with —N(H)—, —N(C₁-C₆alkyl)-, —N(C₆-C₁₄aryl)-, —S—, —SO—, —S(O)₂₋, or —O—;

R² and R³ are each independently selected from: a) C₁-C₈acyl-, b) C₁-C₆alkyl-, which is optionally substituted with from 1 to 3 substituents independently selected from: i) H₂N—, ii) (C₁-C₆alkyl)amino-, iii) di(C₁-C₆alkyl)amino-, and iv) C₁-C₉heterocyclyl-, c) (C₁-C₆alkyl)amido-, d) (C₁-C₆alkyl)carboxyl-, e) (C₁-C₆alkyl)carbonylamido-, f) C₁-C₆alkoxy- optionally substituted by C₁-C₆alkoxy- or C₁-C₉heteroaryl-, g) (C₁-C₆alkoxy)carbonyl-, h) (C₆-C₁₄aryl)alkyl-O—, wherein the ring portion of the (C₆-C₁₄aryl)alkyl-O— group is optionally substituted with from 1 to 3 substituents independently selected from: i) C₁-C₆alkoxy-, and ii) halogen, i) C₃-C₈cycloalkyl-, j) halogen, k) C₁-C₆haloalkyl-, l) C₁-C₉heterocyclyl- optionally substituted by C₁-C₆alkyl- or C₁-C₆hydroxylalkyl-, m) C₁-C₉heterocyclyl(C₁-C₆alkyl)- optionally substituted by C₁-C₆alkyl-, n) hydroxyl, o) C₁-C₆hydroxylalkyl-, p) C₁-C₆perfluoroalkyl-, q) C₁-C₆perfluoroalkyl-O—, r) R⁶R⁷N—, s) C₁-C₉heterocyclyl-, t) CN, u) HO₂C—, v) R⁶R⁷NC(O)—, w) C₁-C₉heterocyclyl-C(O)—, x) R⁶C(O)NH—, y) R⁶R⁷NS(O)₂—, z) R⁶R⁷NC(O)NHC(O)NH—, aa) R⁸OC(O)NHC(O)NH—, bb) C₁-C₆alkoxy-C₁-C₆alkylene-NH—C₁-C₆alkylene-, cc) C₁-C₆hydroxylalkyl-NH—C₁-C₆alkylene-, dd) amino(C₁-C₆alkyl)-NH—C₁-C₆alkylene-, ee) di(C₁-C₆alkyl)amino-C₁-C₆alkylene-NH—C₁-C₆alkylene-, C₁— ff) C₆hydroxylalkyl-NH—, gg) amino(C₁-C₆alkyl)-NH—, hh) (C₁-C₆alkyl) N-alkylamido-, ii) R⁶R⁷NC(O)NH—, jj) C₁-C₉heterocyclyl-C(O)NH—, kk) R⁸OC(O)NH—, ll) R⁸S(O)₂NH—, mm) R⁸S(O)₂—, nn) —C(═N—(OR⁶))—(NR⁶R⁷), or oo) O₂N—;

R⁶ and R⁷ are each independently selected from: H; C₁-C₆alkyl- optionally substituted with from 1 to 3 substituents independently selected from C₁-C₆alkoxy-, H₂N—, (C₁-C₆alkyl)amino-, di(C₁-C₆alkyl)amino-, C₆-C₁₄aryl-, C₁-C₉heterocyclyl- optionally substituted by C₁-C₆alkyl-, and C₁-C₉heteroaryl-; C₁-C₆alkoxy; C₁-C₉heteroaryl- optionally substituted with from 1 to 3 substituents independently selected from C₁-C₆alkyl- optionally substituted with H₂N—, (C₁-C₆alkyl)amino-, or di(C₁-C₆alkyl)amino-, C₁-C₉heterocyclyl(C₁-C₆alkyl)-, halogen, hydroxyl, H₂N—, O₂N—, H₂NSO₂—, HO₂C—, (C₁-C₆alkoxy)carbonyl-, (C₁-C₆alkoxy)C(O)NH—, (C₁-C₆alkyl)amino-, di(C₁-C₆alkyl)amino-, R⁹R¹⁰NC(O)—, R⁹O—, R⁹R¹⁰N—, R⁹R¹⁰NS(O)₂—, R⁹S(O)₂NR¹⁰—, R⁹R¹⁰NC(O)NH—, R⁹S—, R⁹S(O)—, R⁹S(O)₂—, R⁹C(O)—, C₁-C₉heterocyclyl- optionally substituted by C₁-C₆alkyl- or C₁-C₆hydroxylalkyl-, C₁-C₆hydroxylalkyl-, and perfluoro(C₁-C₆)alkyl-; C₁-C₆hydroxylalkyl-; C₁-C₉heterocyclyl-; C₆-C₁₄aryl- optionally substituted with from 1 to 3 substituents independently selected from C₁-C₆alkyl- optionally substituted with H₂N—, (C₁-C₆alkyl)amino-, or di(C₁-C₆alkyl)amino-, C₁-C₉heterocyclyl(C₁-C₆alkyl)-, halogen, hydroxyl, H₂N—, O₂N—, H₂NSO₂—, HO₂C—, (C₁-C₆alkoxy)carbonyl-, (C₁-C₆alkoxy)C(O)NH—, (C₁-C₆alkyl)amino-, di(C₁-C₆alkyl)amino-, R⁹R¹⁰NC(O)—, Z, wherein Z is R⁹O—, R⁹R¹⁰N—, R⁹R¹⁰NS(O)₂—, R⁹S(O)₂NR¹⁷—, R⁹R¹⁰NC(O)NH—, R⁹S—, R⁹S(O)—, R⁹S(O)₂—, R⁹C(O)—, C₁-C_{C}₉heterocyclyl- optionally substituted by C₁-C₆alkyl- or C₁-C₆hydroxylalkyl-, C₁-C₆hydroxylalkyl-, or perfluoro(C₁-C₆)alkyl-; and C₃-C₈cycloalkyl-;

or R⁶ and R⁷, when taken together with the nitrogen to which they are attached, form a 3- to 7-membered heterocycle- wherein up to two of the carbon atoms of the heterocycle- are optionally replaced with —N(H)—, —N(C₁-C₆alkyl)-, —N(C₆-C₁₄aryl)-, —S—, —SO—, —S(O)₂₋, or —O—;

R⁸ is selected from C₁-C₆alkyl-; C₆-C₁₄aryl-; (C₆-C₁₄aryl)alkyl-, optionally substituted by H₂N—; C₁-C₉heterocyclyl-; C₃-C₈cycloalkyl-; C₁-C₆hydroxylalkyl-; and C₁-C₆perfluoroalkyl-;

R⁹ and R¹⁰ are each independently selected from: H; C₁-C₆alkyl-; (C₁-C₆alkyl)amino-C₂-C₆alkylene-; di(C₁-C₆alkyl)amino-C₂-C₆alkylene-; C₂-C₆alkenyl; C₂-C₆alkynyl; C₆-C₁₄aryl-; (C₆-C₁₄aryl)alkyl-; C₃-C₈cycloalkyl-; C₁-C₆hydroxylalkyl-; C₁-C₉heteroaryl-; (C₁-C₉heteroaryl)alkyl-; C₁-C₉heterocyclyl-; and C₁-C₉heterocyclyl(C₁-C₆alkyl)-;

or R⁹ and R¹⁰, when taken together with the nitrogen to which they are attached, form a 3- to 7-membered heterocycle- wherein up to two of the carbon atoms of the heterocycle- are optionally replaced with —N(H)—, —N(C₁-C₆alkyl)-, —N(C₆-C₁₄aryl)-, —S—, —SO—, —S(O)₂₋, or —O— and the heterocycle is optionally substituted by H₂N—, (C₁-C₆alkyl)amino-, or di(C₁-C₆alkyl)amino-;

m and n are each independently 0, 1, 2, or 3;

except that 2-[(1-methyl-6-phenyl-1H-pyrazolo[3,4-d]pyrimidin-4-yl)amino]-benzoic acid, 2-[(1-ethyl-6-phenyl-1H-pyrazolo[3,4-d]pyrimidin-4-yl)amino]-benzoic acid, 2-[(1-propyl-6-phenyl-1H-pyrazolo[3,4-d]pyrimidin-4-yl)amino]-benzoic acid, 2-[(1-methyl-6-phenyl-1H-pyrazolo[3,4-d]pyrimidin-4-yl)amino]-benzoic acid methyl ester, 2-[(1-methyl-6-phenyl-1H-pyrazolo[3,4-d]pyrimidin-4-yl)amino]-benzoic acid ethyl ester, 1-methyl-6-phenyl-4-(phenylthio)-1H-pyrazolo[3,4-d]pyrimidine, 1-ethyl-6-phenyl-4-(phenylthio)-1H-pyrazolo[3,4-d]pyrimidine, and 1-propyl-6-phenyl-4-(phenylthio)-1H-pyrazolo[3,4-d]pyrimidine are excluded;

and when R¹ is phenyl, X is —NH—, and m is 2, then (R²)ₙ is not monofluoro.

In one aspect, the invention provides compounds of the Formula II:

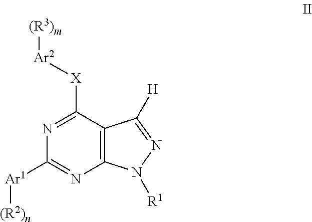

or a pharmaceutically acceptable salt thereof wherein; the constituent variables are as defined above for Formula I.

In one embodiment, X is —NH—.

In one aspect, the invention provides compounds of the Formula III:

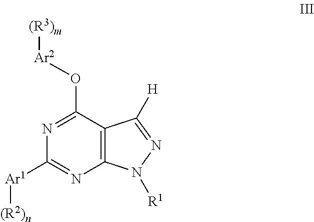

or pharmaceutically acceptable salts thereof, wherein the constituent variables are as defined above for Formula I.

Illustrative compounds of Formula III are set forth below:
N-(3,4-dimethoxyphenyl)-N,1-dimethyl-6-phenyl-1H-pyrazolo[3,4-d]pyrimidin-4-amine;
N-(4-methoxyphenyl)-N,1-dimethyl-6-phenyl-1H-pyrazolo[3,4-d]pyrimidin-4-amine;
4-(1H-indol-5-yloxy)-6-(3-methoxyphenyl)-1-methyl-1H-pyrazolo[3,4-d]pyrimidine;
6-(3,4-dimethoxyphenyl)-4-(1H-indol-5-yloxy)-1-methyl-1H-pyrazolo[3,4-d]pyrimidine;
4-[4-(1H-indol-5-yloxy)-1-methyl-1H-pyrazolo[3,4-d]pyrimidin-6-yl]-N,N-dimethylaniline;
4-(1H-indol-5-yloxy)-1-methyl-6-pyridin-3-yl-1H-pyrazolo[3,4-d]pyrimidine;
4-(1H-indol-5-yloxy)-1-methyl-6-[3-(trifluoromethyl)phenyl]-1H-pyrazolo[3,4-d]pyrimidine;
3-[4-(1H-indol-5-yloxy)-1-methyl-1H-pyrazolo[3,4-d]pyrimidin-6-yl]benzonitrile;
6-biphenyl-3-yl-4-(1H-indol-5-yloxy)-1-methyl-1H-pyrazolo[3,4-d]pyrimidine;
4-(3,4-dimethoxyphenoxy)-6-(3-methoxyphenyl)-1-methyl-1H-pyrazolo[3,4-d]pyrimidine;
4-(3,4-dimethoxyphenoxy)-6-(3,4-dimethoxyphenyl)-1-methyl-1H-pyrazolo[3,4-d]pyrimidine;
4-[4-(3,4-dimethoxyphenoxy)-1-methyl-1H-pyrazolo[3,4-d]pyrimidin-6-yl]-N,N-dimethylaniline;
6-biphenyl-3-yl-4-(3,4-dimethoxyphenoxy)-1-methyl-1H-pyrazolo[3,4-d]pyrimidine;
N-(4-{[6-(3-methoxyphenyl)-1-methyl-1H-pyrazolo[3,4-d]pyrimidin-4-yl]oxy}phenyl)acetamide;
N-(4-{[6-(3-hydroxyphenyl)-1-methyl-1H-pyrazolo[3,4-d]pyrimidin-4-yl]oxy}phenyl)acetamide;
N-[4-({6-[4-(dimethylamino)phenyl]-1-methyl-1H-pyrazolo[3,4-d]pyrimidin-4-yl}oxy)phenyl]acetamide;
N-[4-({1-methyl-6-[3-(trifluoromethyl)phenyl]-1H-pyrazolo[3,4-d]pyrimidin-4-yl}oxy)phenyl]acetamide;
N-{4-[(6-biphenyl-3-yl-1-methyl-1H-pyrazolo[3,4-d]pyrimidin-4-yl)oxy]phenyl}acetamide;
N-(4-{[6-(4-hydroxyphenyl)-1-methyl-1H-pyrazolo[3,4-d]pyrimidin-4-yl]oxy}phenyl)acetamide;
N-(4-{[6-(1H-indol-5-yl)-1-methyl-1H-pyrazolo[3,4-d]pyrimidin-4-yl]oxy}phenyl)acetamide;
6-biphenyl-4-yl-4-(1H-indol-5-yloxy)-1-methyl-1H-pyrazolo[3,4-d]pyrimidine;
4-(1H-indol-5-yloxy)-6-(6-methoxy-2-naphthyl)-1-methyl-1H-pyrazolo[3,4-d]pyrimidine;
4-(1H-indol-5-yloxy)-6-(4-isopropylphenyl)-1-methyl-1H-pyrazolo[3,4-d]pyrimidine;
4-(1H-indol-5-yloxy)-1-methyl-6-(1-methyl-1H-pyrazol-4-yl)-1H-pyrazolo[3,4-d]pyrimidine;
6-(3-chlorophenyl)-4-(1H-indol-5-yloxy)-1-methyl-1H-pyrazolo[3,4-d]pyrimidine;
4-(1H-indol-5-yloxy)-1-methyl-6-(3-nitrophenyl)-1H-pyrazolo[3,4-d]pyrimidine;
6-(3-furyl)-4-(1H-indol-5-yloxy)-1-methyl-1H-pyrazolo[3,4-d]pyrimidine;
6-{3-[(2-chlorobenzyl)oxy]phenyl}-4-(1H-indol-5-yloxy)-1-methyl-1H-pyrazolo[3,4-d]pyrimidine;
6-{4-[(3,5-dimethoxybenzyl)oxy]phenyl}-4-(1H-indol-5-yloxy)-1-methyl-1H-pyrazolo[3,4-d]pyrimidine;
6-{3-[(3,5-dimethoxybenzyl)oxy]phenyl}-4-(1H-indol-5-yloxy)-1-methyl-1H-pyrazolo[3,4-d]pyrimidine;
6-(3,5-dimethylisoxazol-4-yl)-4-(1H-indol-5-yloxy)-1-methyl-1H-pyrazolo[3,4-d]pyrimidine;
4-(3,4-dimethoxyphenoxy)-1-methyl-6-phenyl-1H-pyrazolo[3,4-d]pyrimidine;
N-{4-[(1-methyl-6-phenyl-1H-pyrazolo[3,4-d]pyrimidin-4-yl)oxy]phenyl}acetamide;
4-[(1-methyl-6-phenyl-1H-pyrazolo[3,4-d]pyrimidin-4-yl)oxy]benzamide; and
4-(1H-indol-5-yloxy)-1-methyl-6-phenyl-1H-pyrazolo[3,4-d]pyrimidine; or a pharmaceutically acceptable salt thereof.

In one aspect, the invention provides compounds of the Formula IV:

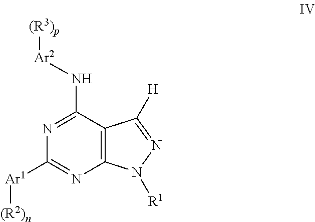

or pharmaceutically acceptable salts thereof, wherein:

Ar² is phenyl or indolyl;

R³ is independently selected from: a) C₁-C₈acyl-, b) C₁-C₆alkyl-, which is substituted with from 1 to 3 substituents independently selected from: i) H₂N—, ii) (C₁-C₆alkyl)amino-, iii) di(C₁-C₆alkyl)amino-, and iv) C₁-C₉heterocyclyl-, c) (C₁-C₆alkyl)amido-, d) (C₁-C₆alkyl)carboxyl-, e) (C₁-C₆alkyl)carbonylamido-, f) C₁-C₆alkoxy- optionally substituted by C₁-C₆alkoxy- or C₁-C₉heteroaryl-, g) (C₁-C₆alkoxy)carbonyl-, h) (C₆-C₁₄aryl)alkyl-O—, wherein the ring portion of the (C₆-C₁₄aryl)alkyl-O— group is optionally substituted with from 1 to 3 substituents independently selected from: i) C₁-C₆alkoxy-, and ii) halogen, i) C₃-C₈cycloalkyl-, j) C₁-C₆haloalkyl-, k) C₁-C₉heterocyclyl- optionally substituted by C₁-C₆alkyl- or C₁-C₆hydroxylalkyl-, l) C₁-C₉heterocyclyl(C₁-C₆alkyl)- optionally substituted by C₁-C₆alkyl-, m) hydroxyl, n) C₁-C₆hydroxylalkyl-, o) R⁶R⁷N—, p) C₁-C₉heterocyclyl-, q) CN, r) HO₂C—, s) H₂NC(O)— t) C₁-C₉heterocyclyl-C(O)—, u) R⁶C(O)NH—, v) R⁶R⁷NS(O)₂—, w) R⁶R⁷NC(O)NHC(O)NH—, x) R⁸OC(O)NHC(O)NH—, y) C₁-C₆alkoxy-C₁-C₆alkylene-NH—C₁-C₆alkylene-, z) C₁-C₆hydroxylalkyl-NH—C₁-C₆alkylene-, aa) amino(C₁-C₆alkyl)-NH—C₁-C₆alkylene-, bb) di(C₁-C₆alkyl)amino-C₁-C₆alkylene-NH—C₁-C₆alkylene-, cc) C₁-C₆hydroxylalkyl-NH—, dd) amino(C₁-C₆alkyl)-NH—, ee) (C₁-C₆alkyl) N-alkylamido-, ff) R⁶R⁷NC(O)NH—, gg)C₁-C₉heterocyclyl-C(O)NH—, hh) R⁸OC(O)NH—, ii) R⁸S(O)₂NH—, jj) R⁸S(O)₂—, kk) —C(═N—(OR⁶))—(NR⁶R⁷), or ll) O₂N—;

p is 1, 2, or 3;

and the remaining constituent variables are as defined above for Formula I.

Illustrative compounds of Formula IV are set forth below:
4-{[1-(1-benzylpiperidin-4-yl)-6-phenyl-1H-pyrazolo[3,4-d]pyrimidin-4-yl]amino}benzamide;
1-(1-benzylpiperidin-4-yl)-N-(3,4-dimethoxyphenyl)-6-phenyl-1H-pyrazolo[3,4-d]pyrimidin-4-amine;
3-{[1-(1-benzylpiperidin-4-yl)-6-phenyl-1H-pyrazolo[3,4-d]pyrimidin-4-yl]amino}benzamide;
1-(1-benzylpiperidin-4-yl)-N-1H-indol-5-yl-6-phenyl-1H-pyrazolo[3,4-d]pyrimidin-4-amine;
N-(3-{[1-(1-benzylpiperidin-4-yl)-6-phenyl-1H-pyrazolo[3,4-d]pyrimidin-4-yl]amino}phenyl)acetamide;
4-{[1-(1-benzylpiperidin-4-yl)-6-(3-hydroxyphenyl)-1H-pyrazolo[3,4-d]pyrimidin-4-yl]amino}benzamide;
3-{1-(1-benzylpiperidin-4-yl)-4-[(3,4-dimethoxyphenyl)amino]-1H-pyrazolo[3,4-d]pyrimidin-6-yl]phenol;
3-{[1-(1-benzylpiperidin-4-yl)-6-(3-hydroxyphenyl)-1H-pyrazolo[3,4-d]pyrimidin-4-yl]amino}benzamide;
3-[1-(1-benzylpiperidin-4-yl)-4-(1H-indol-5-ylamino)-1H-pyrazolo[3,4-d]pyrimidin-6-yl]phenol;
N-(3-{[1-(1-benzylpiperidin-4-yl)-6-(3-hydroxyphenyl)-1H-pyrazolo[3,4-d]pyrimidin-4-yl]amino}phenyl)acetamide;
4-[(1-methyl-6-phenyl-1H-pyrazolo[3,4-d]pyrimidin-4-yl)amino]benzamide;
4-[(6-phenyl-1-piperidin-4-yl-1H-pyrazolo[3,4-d]pyrimidin-4-yl)amino]benzamide;
N-(3,4-dimethoxyphenyl)-6-phenyl-1-piperidin-4-yl-1H-pyrazolo[3,4-d]pyrimidin-4-amine;
3-[(6-phenyl-1-piperidin-4-yl-1H-pyrazolo[3,4-d]pyrimidin-4-yl)amino]benzamide;
N-{3-[(6-phenyl-1-piperidin-4-yl-1H-pyrazolo[3,4-d]pyrimidin-4-yl)amino]phenyl}acetamide;
4-{[6-(3-hydroxyphenyl)-1-piperidin-4-yl-1H-pyrazolo[3,4-d]pyrimidin-4-yl]amino}benzamide;
3-{4-[(3,4-dimethoxyphenyl)amino]-1-piperidin-4-yl-1H-pyrazolo[3,4-d]pyrimidin-6-yl}phenol;
3-{[6-(3-hydroxyphenyl)-1-piperidin-4-yl-1H-pyrazolo[3,4-d]pyrimidin-4-yl]amino}benzamide;
3-[4-(1H-indol-5-ylamino)-1-piperidin-4-yl-1H-pyrazolo[3,4-d]pyrimidin-6-yl]phenyl;
N-(3-{[6-(3-hydroxyphenyl)-1-piperidin-4-yl-1H-pyrazolo[3,4-d]pyrimidin-4-yl]amino}phenyl)acetamide;
4-{[1-(1-benzylpiperidin-4-yl)-6-(3-nitrophenyl)-1H-pyrazolo[3,4-d]pyrimidin-4-yl]amino}benzamide;
4-({1-(1-benzylpiperidin-4-yl)-6-[4-(hydroxymethyl)phenyl]-1H-pyrazolo[3,4-d]pyrimidin-4-yl}amino)benzamide;
4-{[1-(1-benzylpiperidin-4-yl)-6-(3-methoxyphenyl)-1H-pyrazolo[3,4-d]pyrimidin-4-yl]amino}benzamide;
4-{[1-(1-benzylpiperidin-4-yl)-6-(4-hydroxyphenyl)-1H-pyrazolo[3,4-d]pyrimidin-4-yl]amino}benzamide;
4-({1-(1-benzylpiperidin-4-yl)-6-[3-(trifluoromethyl)phenyl]-1H-pyrazolo[3,4-d]pyrimidin-4-yl}amino)benzamide;
4-{[1-(1-benzylpiperidin-4-yl)-6-(2-hydroxyphenyl)-1H-pyrazolo[3,4-d]pyrimidin-4-yl]amino}benzamide;
1-(1-benzylpiperidin-4-yl)-N-(3,4-dimethoxyphenyl)-6-(3-nitrophenyl)-1H-pyrazolo[3,4-d]pyrimidin-4-amine;
(4-{1-(1-benzylpiperidin-4-yl)-4-[(3,4-dimethoxyphenyl)amino]-1H-pyrazolo[3,4-d]pyrimidin-6-yl}phenyl)methanol;
1-(1-benzylpiperidin-4-yl)-N-(3,4-dimethoxyphenyl)-6-(3-methoxyphenyl)-1H-pyrazolo[3,4-d]pyrimidin-4-amine;
4-{1-(1-benzylpiperidin-4-yl)-4-[(3,4-dimethoxyphenyl)amino]-1H-pyrazolo[3,4-d]pyrimidin-6-yl}phenol;
1-(1-benzylpiperidin-4-yl)-N-(3,4-dimethoxyphenyl)-6-[3-(trifluoromethyl)phenyl]-1H-pyrazolo[3,4-d]pyrimidin-4-amine;
2-{1-(1-benzylpiperidin-4-yl)-4-[(3,4-dimethoxyphenyl)amino]-1H-pyrazolo[3,4-d]pyrimidin-6-yl}phenol;
N,1-dimethyl-N,6-diphenyl-1H-pyrazolo[3,4-d]pyrimidin-4-amine;
4-[methyl(1-methyl-6-phenyl-1H-pyrazolo[3,4-d]pyrimidin-4-yl)amino]benzamide;
N-{4-[methyl(1-methyl-6-phenyl-1H-pyrazolo[3,4-d]pyrimidin-4-yl)amino]phenyl}acetamide;
4-[(6-biphenyl-4-yl-1-methyl-1H-pyrazolo[3,4-d]pyrimidin-4-yl)amino]benzamide;
4-{[6-(4-isopropylphenyl)-1-methyl-1H-pyrazolo[3,4-d]pyrimidin-4-yl]amino}benzamide;
4-{[1-methyl-6-(1-methyl-1H-pyrazol-4-yl)-1H-pyrazolo[3,4-d]pyrimidin-4-yl]amino}benzamide;
4-{[6-(3,5-dimethylisoxazol-4-yl)-1-methyl-1H-pyrazolo[3,4-d]pyrimidin-4-yl]amino}benzamide;
4-{[6-(3-chlorophenyl)-1-methyl-1H-pyrazolo[3,4-d]pyrimidin-4-yl]amino}benzamide;
4-{[6-(3-furyl)-1-methyl-1H-pyrazolo[3,4-d]pyrimidin-4-yl]amino}benzamide;
4-(1-methyl-6-(4-(3-methylureido)phenyl)-1H-pyrazolo[3,4-d]pyrimidin-4-ylamino)benzamide; and
4-(1-methyl-6-(3-(3-methylureido)phenyl)-1H-pyrazolo[3,4-d]pyrimidin-4-ylamino)benzamide; or a pharmaceutically acceptable salt thereof.

In one aspect, the invention provides compounds of the Formula V:

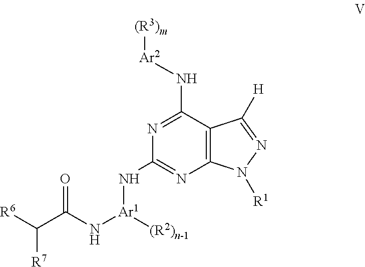

or a pharmaceutically acceptable salt thereof, wherein (n-1) is 0, 1, or 2 and the remaining constituent variables are as defined above for Formula I.

In one embodiment, Ar¹ and Ar² are phenyl.

In one embodiment, R¹ is C₁-C₆alkyl.

In one embodiment, R¹ is CH₃.

In one embodiment, (n-1) is 0.

In one embodiment, m is 1.

In one embodiment, R³ is H₂NC(O)—.

In one embodiment, R⁶ is C₆-C₁₄aryl- substituted with R⁹R¹⁰NC(O)—.

Illustrative compounds of Formula V are set forth below:
4-{[1-Methyl-6-({4-[(pyridin-3-ylcarbamoyl)amino]phenyl}amino)-1H-pyrazolo[3,4-d]pyrimidin-4-yl]amino}benzamide;
4-{[1-Methyl-6-({3-[(pyridin-3-ylcarbamoyl)amino]phenyl}amino)-1H-pyrazolo[3,4-d]pyrimidin-4-yl]amino}benzamide;
4-{[1-Methyl-6-({4-[({4-[(4-methylpiperazin-1-yl)carbonyl]phenyl}carbamoyl)amino]phenyl}amino)-1H-pyrazolo[3,4-d]pyrimidin-4-yl]amino}benzamide;
4-({6-[(4-{[(4-{[4-(Dimethylamino)piperidin-1-yl]carbonyl}phenyl)carbamoyl]amino}phenyl)amino]-1-methyl-1H-pyrazolo[3,4-d]pyrimidin-4-yl}amino)benzamide;
4-({6-[(4-{[(4-{[3-(Dimethylamino)pyrrolidin-1-yl]carbonyl}phenyl)carbamoyl]amino}phenyl)amino]-1-methyl-1H-pyrazolo[3,4-d]pyrimidin-4-yl}amino)benzamide;
4-[(1-Methyl-6-{[4-({[4-(morpholin-4-ylcarbonyl)phenyl]carbamoyl}amino)phenyl]amino}-1H-pyrazolo[3,4-d]pyrimidin-4-yl)amino]benzamide;
4-({[4-({4-[(4-Carbamoylphenyl)amino]-1-methyl-1H-pyrazolo[3,4-d]pyrimidin-6-yl}amino)phenyl]carbamoyl}amino)-N-(2-hydroxyethyl)-N-methylbenzamide;
4-[(6-{[4-({[2-(Dimethylamino)ethyl]carbamoyl}amino)phenyl]amino}-1-methyl-1H-pyrazolo[3,4-d]pyrimidin-4-yl)amino]benzamide;
4-({1-Methyl-6-[(4-{[(4-{[4-(propan-2-yl)piperazin-1-yl]carbonyl}phenyl)carbamoyl]amino}phenyl)amino]-1H-pyrazolo[3,4-d]pyrimidin-4-yl}amino)benzamide;
4-{[6-({4-[(Cyclopropylcarbamoyl)amino]phenyl}amino)-1-methyl-1H-pyrazolo[3,4-d]pyrimidin-4-yl]amino}benzamide;
4-{[1-Methyl-6-({4-[(propan-2-ylcarbamoyl)amino]phenyl}amino)-1H-pyrazolo[3,4-d]pyrimidin-4-yl]amino}benzamide;
N-Methyl-4-{[1-methyl-6-({4-[(pyridin-3-ylcarbamoyl)amino]phenyl}amino)-1H-pyrazolo[3,4-d]pyrimidin-4-yl]amino}benzamide;
4-({1-Methyl-6-[(4-{[(1-methylpiperidin-4-yl)carbamoyl]amino}phenyl)amino]-1H-pyrazolo[3,4-d]pyrimidin-4-yl}amino)benzamide;
4-({[4-({4-[(4-Carbamoylphenyl)amino]-1-methyl-1H-pyrazolo[3,4-d]pyrimidin-6-yl]amino)phenyl]carbamoyl}amino)-N-[2-(dimethylamino)ethyl]-N-methylbenzamide;
N,N-Dimethyl-4-{[1-methyl-6-({4-[(pyridin-3-ylcarbamoyl)amino]phenyl}amino)-1H-pyrazolo[3,4-d]pyrimidin-4-yl]amino}benzamide;
1-[4-({1-Methyl-4-[(1-oxo-2,3-dihydro-1H-isoindol-5-yl)amino]-1H-pyrazolo[3,4-d]pyrimidin-6-yl}amino)phenyl]-3-pyridin-3-ylurea; and
1-[4-({1-Methyl-4-[(2-methyl-1-oxo-2,3-dihydro-1H-isoindol-5-yl)amino]-1H-pyrazolo[3,4-d]pyrimidin-6-yl}amino)phenyl]-3-pyridin-3-ylurea; or a pharmaceutically acceptable salt thereof.

In other aspects, the invention provides pharmaceutical compositions comprising compounds or pharmaceutically acceptable salts of the compounds of any of the present Formulas I-V and a pharmaceutically acceptable carrier.

In other aspects, the invention provides that the pharmaceutically acceptable carrier suitable for oral administration and the composition comprises an oral dosage form.

In other aspects, the invention provides a composition comprising a compound of any of the Formulas I-V; a second compound selected from the group consisting of a topoisomerase I inhibitor, a MEK½ inhibitor, a HSP90 inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, hydroxyzine, glatiramer acetate, interferon beta-1a, interferon beta-1b, natalizumab, and lavendustin A; and a pharmaceutically acceptable carrier.

In other aspects, the second compound is Avastin.

In other aspects, the invention provides a method of treating a PI3K-related disorder, comprising administering to a mammal in need thereof a compound of any of the Formulas I-V in an amount effective to treat a PI3K-related disorder.

In other aspects, the PI3K-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

In other aspects, the PI3K-related disorder is cancer.

In other aspects, the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

In other aspects, the invention provides a method of treating an mTOR-related disorder, comprising administering to a mammal in need thereof a compound of any of the Formulas I-V in an amount effective to treat an mTOR-related disorder.

In other aspects, the mTOR-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

In other aspects, the mTOR-related disorder is cancer.

In other aspects, the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

In other aspects, the invention provides a method of treating advanced renal cell carcinoma, comprising administering to a mammal in need thereof a compound of any of the Formulas I-V in an amount effective to treat advanced renal cell carcinoma.

In other aspects, the invention provides a method of treating acute lymphoblastic leukemia, comprising administering to a mammal in need thereof a compound of any of the Formulas I-V in an amount effective to treat acute lymphoblastic leukemia.

In other aspects, the invention provides a method of treating acute malignant melanoma, comprising administering to a mammal in need thereof a compound of any of the Formulas I-V in an amount effective to treat malignant melanoma.

In other aspects, the invention provides a method of treating soft-tissue or bone sarcoma, comprising administering to a mammal in need thereof a compound of any of the Formulas I-V in an amount effective to treat soft-tissue or bone sarcoma.

In other aspects, the invention provides a method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof a composition comprising a compound of any of the Formulas I-V; a second compound selected from the group consisting of a topoisomerase I inhibitor, a MEK½ inhibitor, a HSP90 inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, hydroxyzine, glatiramer acetate, interferon beta-1a, interferon beta-1b, natalizumab, and lavendustin A; and a pharmaceutically acceptable carrier. in an amount effective to treat the cancer.

In other aspects, the invention provides a method of inhibiting mTOR in a subject, comprising administering to a subject in need thereof a compound of any of the Formulas I-V in an amount effective to inhibit mTOR.

In other aspects, the invention provides a method of inhibiting PI3K in a subject, comprising administering to a subject in need thereof a compound of any of the Formulas I-V in an amount effective to inhibit PI3K.

In other aspects, the invention provides a method of inhibiting mTOR and PI3K together in a subject, comprising administering to a subject in need thereof a compound of any of the Formulas I-V in an amount effective to inhibit mTOR and PI3K.

In other aspects, the invention provides a method of synthesizing a compound of Formula II, comprising:

reacting the 6-chloro-1H-pyrazolo[3,4-d]pyrimidine compound XV with the boronic acid R²—Ar¹(B(OH)₂

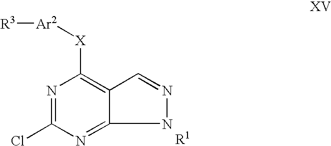

and a suitable catalyst, wherein Ar¹, Ar², X, and R¹-R³ are as defined above in formula I, thereby producing a compound of formula II:

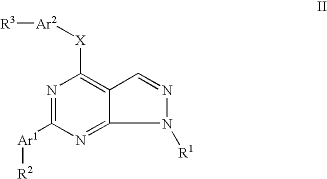

or a pharmaceutically acceptable salt thereof.

The method in which the compound of formula XV is prepared by a process comprising reacting a 4,6-dichloro-1H-pyrazolo[3,4-d]pyrimidine intermediate of formula VIII:

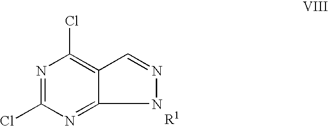

with phenols, arylmercaptans, heteroarylmercaptans, heteroarylamines, or anilines of the formula R³—Ar²—XH, thereby providing a mono chloro derivative of formula XV.

Representative “pharmaceutically acceptable salts” include but are not limited to, e.g., water-soluble and water-insoluble salts, such as the acetate, aluminum, amsonate (4,4-diaminostilbene-2,2-disulfonate), benzathine (N,N′-dibenzylethylenediamine), benzenesulfonate, benzoate, bicarbonate, bismuth, bisulfate, bitartrate, borate, bromide, butyrate, calcium, calcium edetate, camsylate (camphorsulfonate), carbonate, chloride, choline, citrate, clavulariate, diethanolamine, dihydrochloride, diphosphate, edetate, edisylate (camphorsulfonate), esylate (ethanesulfonate), ethylenediamine, fumarate, gluceptate (glucoheptonate), gluconate, glucuronate, glutamate, hexafluorophosphate, hexylresorcinate, hydrabamine (N,N′-bis(dehydroabietyl)ethylenediamine), hydrobromide, hydrochloride, hydroxynaphthoate, 1-hydroxy-2-naphthoate, 3-hydroxy-2-naphthoate, iodide, isothionate (2-hydroxyethanesulfonate), lactate, lactobionate, laurate, lauryl sulfate, lithium, magnesium, malate, maleate, mandelate, meglumine (1-deoxy-1-(methylamino)-D-glucitol), mesylate, methyl bromide, methylnitrate, methylsulfate, mucate, napsylate, nitrate, N-methylglucamine ammonium salt, oleate, oxalate, palmitate, pamoate (4,4′-methylenebis-3-hydroxy-2-naphthoate, or embonate), pantothenate, phosphate, picrate, polygalacturonate, potassium, propionate, p-toluenesulfonate, salicylate, sodium, stearate, subacetate, succinate, sulfate, sulfosaliculate, suramate, tannate, tartrate, teoclate (8-chloro-3,7-dihydro-1,3-dimethyl-1H-purine-2,6-dione), triethiodide, tromethamine (2-amino-2-(hydroxymethyl)-1,3-propanediol), valerate, and zinc salts.

Some compounds within the present invention possess one or more chiral centers, and the present invention includes each separate enantiomer of such compounds as well as mixtures of the enantiomers. Where multiple chiral centers exist in compounds of the present invention, the invention includes each combination as well as mixtures thereof. All chiral, diastereomeric, and racemic forms of a structure are intended, unless the specific stereochemistry or isomeric form is specifically indicated. It is well known in the art how to prepare optically active forms, such as by resolution of racemic forms or by synthesis from optically active starting materials.

An “effective amount” when used in connection a compound of the present invention of this invention is an amount effective for inhibiting mTOR or PI3K in a subject.

### Definitions

The following definitions are used in connection with the compounds of the present invention unless the context indicates otherwise. In general, the number of carbon atoms present in a given group is designated “Cₓ-C_{y}”, where x and y are the lower and upper limits, respectively. For example, a group designated as “C₁-C₆” contains from 1 to 6 carbon atoms. The carbon number as used in the definitions herein refers to carbon backbone and carbon branching, but does not include carbon atoms of the substituents, such as alkoxy substitutions and the like. Unless indicated otherwise, the nomenclature of substituents that are not explicitly defined herein are arrived at by naming from left to right the terminal portion of the functionality followed by the adjacent functionality toward the point of attachment. For example, the substituent “arylalkyloxycabonyl” refers to the group (C₆-C₁₄aryl)-(C₁-C₆alkyl)-O—C(O)—. It is understood that the above definitions are not intended to include impermissible substitution patterns (e.g., methyl substituted with 5 fluoro groups, two hydroxyl groups on a single carbon atom, a hydroxyl group on a non-aromatic double bond). Such impermissible substitution patterns are well known to the skilled artisan. In each of the below groups, when a subgroup is designated with a multiple occurrence, each occurrence is selected independently. For example, in di(C₁-C₆alkyl)amino- e.g. (C₁-C₆alkyl)₂N—, the C₁-C₆alkyl groups can be the same or different.

“Acyl-” refers to a group having a straight, branched, or cyclic configuration or a combination thereof, attached to the parent structure through a carbonyl functionality. Such groups may be saturated or unsaturated, aliphatic or aromatic, and carbocyclic or heterocyclic. The carbon count includes the carbonyl carbon atom. Examples of a C₁-C₈acyl- group include HC(O)—, acetyl-, benzoyl-, p-toluyl, nicotinoyl-, propionyl-, isobutyryl-, oxalyl-, and the like. Lower-acyl- refers to acyl groups containing one to four carbons. An acyl- group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₈acyl-, C₆-C₁₄aryl-, C₁-C₉heteroaryl-, or C₃-C₈cycloalkyl-.

“Alkenyl-” refer to a straight or branched chain unsaturated hydrocarbon containing at least one double bond. Where E- and/or Z-isomers are possible, the term “alkenyl” is intended to include all such isomers. Examples of a C₂-C₆alkenyl- group include, but are not limited to, ethylene, propylene, 1-butylene, 2-butylene, isobutylene, sec-butylene, 1-pentene, 2-pentene, isopentene, penta-1,4-dien-1-yl, 1-hexene, 2-hexene, 3-hexene, and isohexene. An alkenyl- group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₈acyl-, C₆-C₁₄aryl-, C₁-C₉heteroaryl-, and C₃-C₈cycloalkyl-.

“Alkoxy-” refers to the group R—O— where R is an alkyl group, as defined below. Exemplary C₁-C₆alkoxy- groups include but are not limited to methoxy, ethoxy, n-propoxy, 1-propoxy, n-butoxy and t-butoxy. An alkoxy group can be unsubstituted or substituted with one or more of the following groups: halogen, hydroxyl, C₁-C₆alkoxy-, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, C₁-C₆alkoxy-, HO₂C—, (C₁-C₆alkoxy)carbonyl-, C₁-C₈acyl-, C₆-C₁₄aryl-, C₁-C₉heteroaryl-, C₃-C₈cycloalkyl-, C₁-C₆haloalkyl-, C₁-C₆aminoalkyl-, (C₁-C₆alkyl)carboxy-, C₁-C₆carbonylamidoalkyl-, or O₂N—.

“(Alkoxy)carbonyl-” refers to the group alkyl-O—C(O)—. Exemplary (C₁-C₆alkoxy)carbonyl- groups include but are not limited to methoxy, ethoxy, n-propoxy, 1-propoxy, n-butoxy and t-butoxy. An (alkoxy)carbonyl- group can be unsubstituted or substituted with one or more of the following groups: halogen, hydroxyl, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, C₁-C₆alkoxy-, HO₂C—, (C₁-C₆alkoxy)carbonyl-, C₁-C₈acyl-, C₆-C₁₄aryl-, C₁-C₉heteroaryl-, C₃-C₈cycloalkyl-, C₁-C₆haloalkyl-, C₁-C₆aminoalkyl-, (C₁-C₆alkyl)carboxy, C₁-C₆carbonylamidoalkyl-, or O₂N—.

“Alkyl-” refers to a hydrocarbon chain that may be a straight chain or branched chain, containing the indicated number of carbon atoms, for example, a Cₗ-Cₗ₀alkyl- group may have from 1 to 10 (inclusive) carbon atoms in it. In the absence of any numerical designation, “alkyl” is a chain (straight or branched) having 1 to 6 (inclusive) carbon atoms in it. Examples of C₁-C₆alkyl- groups include, but are not limited to, methyl, ethyl, propyl, butyl, pentyl, hexyl, isopropyl, isobutyl, sec-butyl, tert-butyl, isopentyl, neopentyl, and isohexyl. An alkyl- group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₈acyl-, C₆-C₁₄aryl-, C₁-C₉heteroaryl-, C₃-C₈cycloalkyl-, C₁-C₆haloalkyl-, C₁-C₆aminoalkyl-, (C₁-C₆alkyl)carboxy-, C₁-C₆carbonylamidoalkyl-, or O₂N—.

“(Alkyl)amido-” refers to a —NHC(O)— group in which the nitrogen atom of said group is attached to an alkyl group, as defined above. Representative examples of a (C₁-C₆alkyl)amido- group include, but are not limited to, —C(O)NHCH₃, —C(O)NHCH₂CH₃, —C(O)NHCH₂CH₂CH₃, —C(O)NHCH₂CH₂CH₂CH₃, —C(O)NHCH₂CH₂CH₂CH₂CH₃, —C(O)NHCH(CH₃)₂, —C(O)NHCH₂CH(CH₃)₂, —C(O)NHCH(CH₃)CH₂CH₃, —C(O)NH—C(CH₃)₃ and —C(O)NHCH₂C(CH₃)₃.

“(Alkyl)amino-” refers to an NH— group, the nitrogen atom of said group being attached to an alkyl group, as defined above. Representative examples of an (C₁-C₆alkyl)amino- group include, but are not limited to —NHCH₃, —NHCH₂CH₃, —NHCH₂CH₂CH₃, —NHCH₂CH₂CH₂CH₃, —NHCH(CH₃)₂, —NHCH₂CH(CH₃)₂, —NHCH(CH₃)CH₂CH₃ and —NH—C(CH₃)₃. An (alkyl)amino group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₈acyl-, C₆-C₁₄aryl-, C₁-C₉heteroaryl-, C₃-C₈cycloalkyl-, C₁-C₆haloalkyl-, C₁-C₆aminoalkyl-, (C₁-C₆alkyl)carboxy-, C₁-C₆carbonylamidoalkyl-, or O₂N—.

“Alkylcarboxy-” refers to an alkyl group, defined above, attached to the parent structure through the oxygen atom of a carboxyl (C(O)—O—) functionality. Examples of (C₁-C₆alkyl)carboxy- include acetoxy, ethylcarboxy, propylcarboxy, and isopentylcarboxy.

“(Alkyl)carbonylamido-” refers to a —C(O)NH— group in which the carbonyl carbon atom of said group is attached to an alkyl group, as defined above. Representative examples of a (C₁-C₆alkyl)carbonylamido- group include, but are not limited to, —NHC(O)CH₃, —NHC(O)CH₂CH₃, —NHC(O)CH₂CH₂CH₃, —NHC(O)CH₂CH₂CH₂CH₃, —NHC(O)CH₂CH₂CH₂CH₂CH₃, —NHC(O)CH(CH₃)₂, —NHC(O)CH₂CH(CH₃)₂, —NHC(O)CH(CH₃)CH₂CH₃, —NHC(O)—C(CH₃)₃ and —NHC(O)CH₂C(CH₃)₃.

“-Alkylene-”, “-alkenylene-”, and “-alkynylene-” refer to alkyl, alkenyl and alkynyl groups, as defined above, having two points of attachment within a chemical structure. Examples of —C₁-C₆alkylene- include ethylene (—CH₂CH₂—), propylene (—CH₂CH₂CH₂—), and dimethylpropylene (—CH₂C(CH₃)₂CH₂—). Likewise, examples of —C₂-C₆alkenylene- include ethenylene (—CH═CH— and propenylene (—CH═CH—CH₂—). Examples of —C₂-C₆alkynylene- include ethynylene (—C≡C—) and propynylene (—C≡C—CH₂—).

“Alkylthio-” refers to the group R—S— where R is an alkyl group, as defined above, attached to the parent structure through a sulfur atom. Examples of C₁-C₆alkylthio- include methylthio, ethylthio, n-propylthio, i-propylthio, n-butylthio, i-butylthio, s-butylthio, t-butylthio, n-pentylthio and n-hexylthio.

“Alkynyl-” refers to a straight or branched chain unsaturated hydrocarbon containing at least one triple bond. Examples of a C₂-C₆alkynyl- group include, but are not limited to, acetylene, propyne, 1-butyne, 2-butyne, isobutyne, sec-butyne, 1-pentyne, 2-pentyne, isopentyne, penta-1,4-diyn-1-yl, 1-hexyne, 2-hexyne, 3-hexyne, and isohexyne. An alkynyl group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₈acyl-, C₆-C₁₄aryl-, C₁-C₉heteroaryl-, and C₃-C₈cycloalkyl-.

“Amido(aryl)-” refers to an aryl group, as defined below, wherein one of the aryl group's hydrogen atoms has been replaced with one or more H₂NC(O)— groups. Representative examples of an amido(C₆-C₁₄aryl)- group include 2-C(O)NH₂-phenyl, 3-C(O)NH₂-phenyl, 4-C(O)NH₂-phenyl, 1-C(O)NH₂-naphthyl, and 2-C(O)NH₂-naphthyl.

“Aminoalkyl-” refers to an alkyl group, as defined above, wherein one or more of the alkyl group's hydrogen atoms has been replaced with H₂N—. Representative examples of an C₁-C₆aminoalkyl- group include, but are not limited to —CH₂NH₂, —CH₂CH₂NH₂, —CH₂CH₂CH₂NH₂, —CH₂CH₂CH₂CH₂NH₂, —CH₂CH(NH₂)CH₃, —CH₂CH(NH₂)CH₂CH₃, —CH(NH₂)CH₂CH₃, —C(CH₃)₂(CH₂NH₂), —CH₂CH₂CH₂CH₂CH₂NH₂, and —CH₂CH₂CH(NH₂)CH₂CH₃. An aminoalkyl- group can be unsubstituted or substituted with one or two of the following groups: C₁-C₆alkoxy-, C₆-C₁₄aryl-, C₁-C₉heteroaryl-, C₃-C₈cycloalkyl-, and C₁-C₆alkyl-.

Aryl- refers to an aromatic hydrocarbon group. Examples of an C₆-C₁₄aryl- group include, but are not limited to, phenyl, 1-naphthyl, 2-naphthyl, 3-biphen-1-yl, anthryl, tetrahydronaphthyl, fluorenyl, indanyl, biphenylenyl, and acenaphthenyl. An aryl group can be monocyclic or polycyclic as long as at least one ring is aromatic and the point of attachment is at an aromatic carbon atom. An aryl group can be unsubstituted or substituted with one or more of the following groups: C₁-C₆alkyl-, halogen, haloalkyl-, hydroxyl, hydroxyl(C₁-C₆alkyl)-, H₂N—, aminoalkyl-, di(C₁-C₆alkyl)amino-, HO₂C—, (C₁-C₆alkoxy)carbonyl-, (C₁-C₆alkyl)carboxy-, di(C₁-C₆alkyl)amido-, H₂NC(O)—, (C₁-C₆alkyl)amido-, or O₂N—.

“(Aryl)alkyl-” refers to an alkyl group, as defined above, wherein one or more of the alkyl group's hydrogen atoms has been replaced with an aryl group as defined above. (C₆-C₁₄Aryl)alkyl- moieties include benzyl, benzhydryl, 1-phenylethyl, 2-phenylethyl, 3-phenylpropyl, 2-phenylpropyl, 1-naphthylmethyl, 2-naphthylmethyl and the like. An (aryl)alkyl- group can be unsubstituted or substituted with one or more of the following groups: halogen, H₂N—, hydroxyl, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₈acyl-, C₆-C₁₄aryl-, C₁-C₉heteroaryl-, C₃-C₈cycloalkyl-, C₁-C₆haloalkyl-, C₁-C₆aminoalkyl-, (C₁-C₆alkyl)carboxy-, C₁-C₆carbonylamidoalkyl-, or O₂N—.

“(Aryl)amino-” refers to a radical of formula (aryl)-NH—, wherein aryl is as defined above. Examples of (C₆-C₁₄aryl)amino- radicals include, but are not limited to, phenylamino (anilido), 1-naphthylamino, 2-naphthylamino, and the like. An (aryl)amino group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₈acyl-, C₆-C₁₄aryl-, C₁-C₉heteroaryl-, or C₃-C₈cycloalkyl-.

“(Aryl)oxy-” refers to the group Ar—O— where Ar is an aryl group, as defined above. Exemplary (C₆-C₁₄aryl)oxy- groups include but are not limited to phenyloxy, α-naphthyloxy, and β-naphthyloxy. An (aryl)oxy group can be unsubstituted or substituted with one or more of the following groups: C₁-C₆alkyl-, halogen, C₁-C₆haloalkyl-, hydroxyl, C₁-C₆hydroxylalkyl-, H₂N—, C₁-C₆aminoalkyl-, di(C₁-C₆alkyl)amino-, HO₂C—, (C₁-C₆alkoxy)carbonyl-, (C₁-C₆alkyl)carboxy, di(C₁-C₆alkyl)amido-, H₂NC(O)—, (C₁-C₆alkyl)amido-, or O₂N—.

“Cycloalkyl-” refers to a monocyclic saturated hydrocarbon ring. Representative examples of a C₃-C₈cycloalkyl- include, but are not limited to, cyclopropyl, cyclobutyl, cyclopentyl, cyclohexyl, cycloheptyl, and cyclooctyl. A cycloalkyl- can be unsubstituted or independently substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₈acyl-, C₆-C₁₄aryl-, C₁-C₉heteroaryl-, or C₃-C₈cycloalkyl-, C₁-C₆haloalkyl-, C₁-C₆aminoalkyl-, (C₁-C₆alkyl)carboxy-, C₁-C₆carbonylamidoalkyl-, or O₂N—. Additionally, each of any two hydrogen atoms on the same carbon atom of the carbocyclic ring can be replaced by an oxygen atom to form an oxo (═O) substituent or the two hydrogen atoms can be replaced by an alkylenedioxy group so that the alkylenedioxy group, when taken together with the carbon atom to which it is attached, form a 5- to 7-membered heterocycle- containing two oxygen atoms.

“Bicyclic cycloalkyl-” refers to a bicyclic saturated hydrocarbon ring system. Representative examples of a C₆-C₁₀bicyclic cycloalkyl- include, but are not limited to, cis-1-decalinyl, trans 2-decalinyl, cis-4-perhydroindanyl, and trans-7-perhydroindanyl. A bicyclic cycloalkyl- can be unsubstituted or independently substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₈acyl-, C₆-C₁₄aryl-, C₁-C₉heteroaryl-, or C₃-C₈cycloalkyl-, haloalkyl-, aminoalkyl-, (C₁-C₆alkyl)carboxy-, carbonylamidoalkyl-, or O₂N—. Additionally, each of any two hydrogen atoms on the same carbon atom of the bicyclic cycloalkyl- rings can be replaced by an oxygen atom to form an oxo (═O) substituent or the two hydrogen atoms can be replaced by an alkylenedioxy group so that the alkylenedioxy group, when taken together with the carbon atom to which it is attached, form a 5- to 7-membered heterocycle containing two oxygen atoms.

“Carbonylamidoalkyl-” refers to a primary carboxyamide (CONH₂), a secondary carboxyamide (CONHR′) or a tertiary carboxyamide (CONR′R″), where R′ and R″ are the same or different substituent groups selected from C₁-C₆alkyl-, C₂-C₆alkenyl, C₂-C₆alkynyl, C₆-C₁₄aryl-, C₁-C₉heteroaryl-, or C₃-C₈cycloalkyl-, attached to the parent compound by an —C₁-C₆alkylene- group as defined above. Exemplary C₁-C₆carbonylamidoalkyl- groups include but are not limited to NH₂C(O)—CH₂—, CH₃NHC(O)—CH₂CH₂—, (CH₃)₂NC(O)—CH₂CH₂CH₂—, CH₂═CHCH₂NCH(O)—CH₂CH₂CH₂CH₂—, HCCCH₂NHC(O)—CH₂CH₂CH₂CH₂CH₂—, C₆H₅NHC(O)—CH₂CH₂CH₂CH₂CH₂CH₂—, 3-pyridylNHC(O)—CH₂CH(CH₃)CH₂CH₂—, and cyclopropyl-CH₂NHC(O)—CH₂CH₂C(CH₃)₂CH₂—.

“Cycloalkenyl-” refers to non-aromatic carbocyclic rings with one or more carbon-to-carbon double bonds within the ring system. The “cycloalkenyl” may be a single ring or may be multi-ring. Multi-ring structures may be bridged or fused ring structures. Examples of C₃-C₁₀cycloalkenyl- groups include, but are not limited to, cyclopropenyl, cyclobutenyl, cyclopentenyl, cyclohexenyl, 4,4a-octalin-3-yl, and cyclooctenyl. A cycloalkenyl can be unsubstituted or independently substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl- , C₁-C₈acyl-, C₆-C₁₄aryl-, C₁-C₉heteroaryl-, or C₃-C₈cycloalkyl-, C₁-C₆haloalkyl-, C₁-C₆aminoalkyl-, (C₁-C₆alkyl)carboxy-, C₁-C₆carbonylamidoalkyl-, or O₂N— Additionally, each of any two hydrogen atoms on the same carbon atom of the cycloalkenyl rings may be replaced by an oxygen atom to form an oxo (═O) substituent or the two hydrogen atoms may be replaced by an alkylenedioxy group so that the alkylenedioxy group, when taken together with the carbon atom to which it is attached, form a 5- to 7-membered heterocycle containing two oxygen atoms.

“Di(alkyl)amido-” refers to a —NC(O)— group in which the nitrogen atom of said group is attached to two alkyl groups, as defined above. Each alkyl group can be independently selected. Representative examples of a di(C₁-C₆alkyl)amido- group include, but are not limited to, —C(O)N(CH₃)₂, —C(O)N(CH₂CH₃)₂, —C(O)N(CH₃)CH₂CH₃, —C(O)N(CH₂CH₂CH₂CH₃)₂, —C(O)N(CH₂CH₃)CH₂CH₂CH₃, —C(O)N(CH₃)CH(CH₃)₂, —C(O)N(CH₂CH₃)CH₂CH(CH₃)₂, —C(O)N(CH(CH₃)CH₂CH₃)₂, —C(O)N(CH₂CH₃)C(CH₃)₃ and —C(O)N(CH₂CH₃)CH₂C(CH₃)₃.

“Di(alkyl)amino-” refers to a nitrogen atom attached to two alkyl groups, as defined above. Each alkyl group can be independently selected. Representative examples of an di(C₁-C₆alkyl)amino- group include, but are not limited to, —N(CH₃)₂, —N(CH₂CH₃)(CH₃), —N(CH₂CH₃)₂, —N(CH₂CH₂CH₃)₂, —N(CH₂CH₂CH₂CH₃)₂, —N(CH(CH₃)₂)₂, —N(CH(CH₃)₂)(CH₃), —N(CH₂CH(CH₃)₂)₂, —NH(CH(CH₃)CH₂CH₃)₂, —N(C(CH₃)₃)₂, —N(C(CH₃)₃)(CH₃), and —N(CH₃)(CH₂CH₃). The two alkyl groups on the nitrogen atom, when taken together with the nitrogen to which they are attached, can form a 3- to 7-membered nitrogen containing heterocycle wherein up to two of the carbon atoms of the heterocycle can be replaced with —N(H)—, —N(C₁-C₆alkyl)-, —N(C₃-C₈cycloalkyl)-, —N(C₆-C₁₄aryl)-, —N(C₁-C₉heteroaryl)-, —N(C₁-C₆aminoalkyl)-, —N(C₆-C₁₄arylamino)-, —O—, —S—, —S(O)—, or —S(O)₂—.

“Halo” or “halogen” refers to fluorine, chlorine, bromine, or iodine.

“Haloalkyl-” refers to an alkyl group, as defined above, wherein one or more of the hydrogen atoms has been replaced with —F, —Cl, —Br, or —I. Each substitution can be independently selected. Representative examples of an C₁-C₆haloalkyl- group include, but are not limited to, —CH₂F, —CCl₃, —CF₃, CH₂CF₃, —CH₂Cl, —CH₂CH₂Br, —CH₂CH₂I, —CH₂CH₂CH₂F, —CH₂CH₂CH₂Cl, —CH₂CH₂CH₂CH₂Br, —CH₂CH₂CH₂CH₂I, —CH₂CH₂CH₂CH₂CH₂Br, —CH₂CH₂CH₂CH₂CH₂I, —CH₂CH(Br)CH₃, —CH₂CH(Cl)CH₂CH₃, —CH(F)CH₂CH₃ and —C(CH₃)₂(CH₂Cl).

“Heteroaryl-” refers to 5-10-membered mono and bicyclic aromatic groups containing at least one heteroatom selected from oxygen, sulfur and nitrogen, wherein any S can optionally be oxidized, and any N can optionally be quaternized with an C₁-C₆alkyl group. Examples of monocyclic C₁-C₉heteroaryl- radicals include, but are not limited to, oxazinyl, thiazinyl, diazinyl, triazinyl, thiadiazolyl, tetrazinyl, imidazolyl, tetrazolyl, isoxazolyl, furanyl, furazanyl, oxazolyl, thiazolyl, thiophenyl, pyrazolyl, triazolyl, pyrimidinyl, N-pyridyl, 2-pyridyl, 3-pyridyl and 4-pyridyl. Examples of bicyclic C₁-C₉heteroaryl- radicals include but are not limited to, benzimidazolyl, indolyl, isoquinolinyl, benzofuranyl, benzothiophenyl, indazolyl, quinolinyl, quinazolinyl, purinyl, benzisoxazolyl, benzoxazolyl, benzthiazolyl, benzodiazolyl, benzotriazolyl, isoindolyl, and indazolyl. The contemplated heteroaryl- rings or ring systems have a minimum of 5 members. Therefore, for example, C₁heteroaryl- radicals would include but are not limited to tetrazolyl, C₂heteroaryl- radicals include but are not limited to triazolyl, thiadiazolyl, and tetrazinyl, C₉heteroaryl- radicals include but are not limited to quinolinyl and isoquinolinyl. A heteroaryl- group can be unsubstituted or substituted with one or more of the following groups: C₁-C₆alkyl-, halogen, C₁-C₆haloalkyl-, hydroxyl, C₁-C₆hydroxylalkyl-, H₂N—, C₁-C₆aminoalkyl-, di(C₁-C₆alkyl)amino-, —COOH, (C₁-C₆alkoxy)carbonyl-, (C₁-C₆alkyl)carboxy-, di(C₁-C₆alkyl)amido-, H₂NC(O)—, (C₁-C₆alkyl)amido-, or O₂N—.

“(Heteroaryl)alkyl-” refers to an alkyl group, as defined above, wherein one or more of the alkyl group's hydrogen atoms has been replaced with a heteroaryl- group as defined above. Examples of (C₁-C₉heteroaryl)alkyl- moieties include 2-pyridylmethyl, 2-thiophenylethyl, 3-pyridylpropyl, 2-quinolinylmethyl, 2-indolylmethyl, and the like. A (heteroaryl)alkyl- group can be unsubstituted or substituted with one or more of the following groups: halogen, H₂N—, hydroxyl, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₈acyl-, C₆-C₁₄aryl-, C₁-C₉heteroaryl-, C₁-C₆haloalkyl-, C₁-C₆aminoalkyl-, (C₁-C₆alkyl)carboxy-, C₁-C₆carbonylamidoalkyl-, or O₂N—.

“(Heteroaryl)oxy-” refers to the group Het-O— where Het is a heteroaryl- group, as defined above. Exemplary (C₁-C₉heteroaryl)oxy- groups include but are not limited to pyridin-2-yloxy, pyridin-3-yloxy, pyrimidin-4-yloxy, and oxazol-5-yloxy. A (heteroaryl)oxy group can be unsubstituted or substituted with one or more of the following groups: C₁-C₆alkyl-, halogen, C₁-C₆haloalkyl-, hydroxyl, C₁-C₆hydroxylalkyl-, H₂N—, C₁-C₆aminoalkyl-, di(C₁-C₆alkyl)amino-, —COOH, (C₁-C₆alkoxy)carbonyl-, (C₁-C₆alkyl)carboxy-, di(C₁-C₆alkyl)amido-, H₂NC(O)—, (C₁-C₆alkyl)amido-, or O₂N—.

“Heteroatom” refers to a sulfur, nitrogen, or oxygen atom.

“Heterocycle” or “heterocyclyl-” refers to 3-10-membered monocyclic, fused bicyclic, and bridged bicyclic groups containing at least one heteroatom selected from oxygen, sulfur and nitrogen, wherein any S can optionally be oxidized, and any N can optionally be quaternized by a C₁-C₆alkyl group. A heterocycle may be saturated or partially saturated. Exemplary C₁-C₉heterocyclyl- groups include but are not limited to aziridine, oxirane, oxirene, thiirane, pyrroline, pyrrolidine, dihydrofuran, tetrahydrofuran, dihydrothiophene, tetrahydrothiophene, dithiolane, piperidine, 1,2,3,6-tetrahydropyridine-1-yl, tetrahydropyran, pyran, thiane, thiine, piperazine, oxazine, 5,6-dihydro-4H-1,3-oxazin-2-yl, 2,5-diazabicyclo[2.2.1]heptane, 2,5-diazabicyclo[2.2.2]octane, 3,6-diazabicyclo[3.1.1]heptane, 3,8-diazabicyclo[3.2.1]octane, 6-oxa-3,8-diazabicyclo[3.2.1]octane, 7-oxa-2,5-diazabicyclo[2.2.2]octane, 2,7-dioxa-5-azabicyclo[2.2.2]octane, 2-oxa-5-azabicyclo[2.2.1]heptane, 2-oxa-5-azabicyclo[2.2.2]octane, 3,6-dioxa-8-azabicyclo[3.2.1]octane, 3-oxa-6-azabicyclo[3.1.1]heptane, 3-oxa-8-azabicyclo[3.2.1]octane, 5,7-dioxa-2-azabicyclo[2.2.2]octane, 6,8-dioxa-3-azabicyclo[3.2.1]octane, 6-oxa-3-azabicyclo[3.1.1]heptane, 8-oxa-3-azabicyclo[3.2.1]octane, 8-oxa-3-azabicyclo[3.2.1]octan-3-yl, 2-methyl-2,5-diazabicyclo[2.2.1]heptane-5-yl, 1,3,3-trimethyl-6-azabicyclo[3.2.1]oct-6-yl, 4-methyl-3,4-dihydro-2H-1,4-benzoxazin-7-yl, thiazine, dithiane, and dioxane. The contemplated heterocycle rings or ring systems have a minimum of 3 members. Therefore, for example, C₁heterocyclyl- radicals would include but are not limited to oxaziranyl, diaziridinyl, and diazirinyl, C₂heterocyclyl- radicals include but are not limited to aziridinyl, oxiranyl, and diazetidinyl, C₉heterocyclyl- radicals include but are not limited to azecanyl, tetrahydroquinolinyl, and perhydroisoquinolinyl.

“Heterocyclyl(alkyl)-” refers to an alkyl group, as defined above, wherein one or more of the alkyl group's hydrogen atoms has been replaced with a heterocycle group as defined above. C₁-C₉Heterocyclyl(C₁-C₆alkyl)- moieties include 2-pyridylmethyl, 1-piperazinylethyl, 4-morpholinylpropyl, 6-piperazinylhexyl, and the like. A heterocyclyl(alkyl)- group can be unsubstituted or substituted with one or more of the following groups: halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₈acyl-, 4- to 7-membered monocyclic heterocycle, C₆-C₁₄aryl-, C₁-C₉heteroaryl-, or C₃-C₈cycloalkyl-.

“Hydroxylalkyl-” refers to an alkyl group, as defined above, wherein one or more of the alkyl group's hydrogen atoms has been replaced with hydroxyl groups. Examples of C₁-C₆hydroxylalkyl-moieties include, for example, —CH₂OH, —CH₂CH₂OH, —CH₂CH₂CH₂OH, —CH₂CH(OH)CH₂OH, —CH₂CH(OH)CH₃, —CH(CH₃)CH₂OH and higher homologs.

“Hydroxylalkenyl-” refers to an alkenyl group, defined above, and substituted on one or more sp³ carbon atoms with a hydroxyl group. Examples of C₃-C₆hydroxylalkenyl- moieties include chemical groups such as —CH═CHCH₂OH, —CH(CH═CH₂)OH, —CH₂CH═CHCH₂OH, —CH(CH₂CH═CH₂)OH, —CH═CHCH₂CH₂OH, —CH(CH═CHCH₃)OH, —CH═CHCH(CH₃)OH, —CH₂CH(CH═CH₂)OH, and higher homologs.

“Nitrogen-containing heteroaryl-” refers to 5-10-membered mono and bicyclic aromatic groups containing at least one nitrogen atom and optionally additional heteroatoms selected from oxygen and sulfur. Examples of nitrogen-containing monocyclic C₁-C₉heteroaryl- radicals include, but are not limited to, oxazinyl, thiazinyl, diazinyl, triazinyl, tetrazinyl, imidazolyl, tetrazolyl, isoxazolyl, furazanyl, oxazolyl, thiazolyl, pyrazolyl, triazolyl, pyrimidinyl, N-pyridyl, 2-pyridyl, 3-pyridyl and 4-pyridyl. Examples of nitrogen-containing bicyclic C₁-C₉heteroaryl- radicals include but are not limited to, benzimidazolyl, indolyl, isoquinolinyl, indazolyl, quinolinyl, quinazolinyl, purinyl, benzisoxazolyl, benzoxazolyl, benzthiazolyl, benzodiazolyl, benzotriazolyl, isoindolyl and indazolyl. A nitrogen-containing heteroaryl- group can be unsubstituted or substituted with one or more of the following groups: C₁-C₆alkyl-, halogen, C₁-C₆haloalkyl-, hydroxyl, C₁-C₆hydroxylalkyl-, H₂N—, C₁-C₆aminoalkyl-, di(C₁-C₆alkyl)amino-, HO₂C—, (C₁-C₆alkoxy)carbonyl-, (C₁-C₆alkyl)carboxy-, di(C₁-C₆alkyl)amido-, H₂NC(O)—, (C₁-C₆alkyl)amido-, or O₂N—.

“Perfluoroalkyl-” refers to alkyl group, defined above, having two or more fluorine atoms. Examples of a C₁-C₆perfluoroalkyl- group include CF₃, CH₂CF₃, CF₂CF₃ and CH(CF₃)₂.

The term “optionally substituted”, unless otherwise specified, as used herein means that at least one hydrogen atom of the optionally substituted group has been substituted with halogen, H₂N—, (C₁-C₆alkyl)amino-, di(C₁-C₆alkyl)amino-, (C₁-C₆alkyl)C(O)N(C₁-C₃alkyl)-, (C₁-C₆alkyl)carbonylamido-, HC(O)NH—, H₂NC(O)—, (C₁-C₆alkyl)NHC(O)—, di(C₁-C₆alkyl)NC(O)—, —CN, hydroxyl, C₁-C₆alkoxy-, C₁-C₆alkyl-, HO₂C—, (C₁-C₆alkoxy)carbonyl-, C₁-C₈acyl-, C₆-C₁₄aryl-, C₁-C₉heteroaryl-, or C₃-C₈cycloalkyl-.

A “subject” is a mammal, e.g., a human, mouse, rat, guinea pig, dog, cat, horse, cow, pig, or non-human primate, such as a monkey, chimpanzee, baboon or gorilla.

The compounds of the present invention exhibit an mTOR inhibitory activity and, therefore, can be utilized to inhibit abnormal cell growth in which mTOR plays a role. Thus, the compounds of the present invention are effective in the treatment of disorders with which abnormal cell growth actions of mTOR are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovary cancer, prostate cancer, lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

The compounds of the present invention exhibit a PI3 kinase inhibitory activity and, therefore, can be utilized in order to inhibit abnormal cell growth in which PI3 kinases play a role. Thus, the compounds of the present invention are effective in the treatment of disorders with which abnormal cell growth actions of PI3 kinases are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovary cancer, prostate cancer, lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

The compounds of the present invention may inhibit both mTOR and PI3 kinase simultaneously and, therefore, can be utilized in order to inhibit abnormal cell growth in which both mTOR and PI3 kinases simultaneously play a role. Thus, the compounds of the present invention are effective in the treatment of disorders with which abnormal cell growth actions of PI3 kinases are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the compounds of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovary cancer, prostate cancer, lung cancer, colon cancer, pancreas cancer, renal cancer, gastric cancer, brain tumor, advanced renal cell carcinoma, acute lymphoblastic leukemia, malignant melanoma, soft-tissue or bone sarcoma, etc.

For therapeutic use, the pharmacologically active compounds of any of the Formulas I-V will normally be administered as a pharmaceutical composition comprising as the (or an) essential active ingredient at least one such compound in association with a solid or liquid pharmaceutically acceptable carrier and, optionally, with pharmaceutically acceptable adjutants and excipients employing standard and conventional techniques.

The pharmaceutical compositions of this invention include suitable dosage forms for oral, parenteral (including subcutaneous, intramuscular, intradermal and intravenous) bronchial or nasal administration. Thus, if a solid carrier is used, the preparation may be made into tablets, placed in a hard gelatin capsule in powder or pellet form, or in the form of a troche or lozenge. The solid carrier may contain conventional excipients such as binding agents, fillers, lubricants used to make tablets, disintegrants, wetting agents and the like. The tablet may, if desired, be film coated by conventional techniques. If a liquid carrier is employed, the preparation may be in the form of a syrup, emulsion, soft gelatin capsule, sterile vehicle for injection, an aqueous or non-aqueous liquid suspension, or may be a dry product for reconstitution with water or other suitable vehicle before use. Liquid preparations may contain conventional additives such as suspending agents, emulsifying agents, wetting agents, non-aqueous vehicle (including edible oils), preservatives, as well as flavoring and/or coloring agents. For parenteral administration, a vehicle normally will comprise sterile water, at least in large part, although saline solutions, glucose solutions and like may be utilized. Injectable suspensions also may be used, in which case conventional suspending agents may be employed. Conventional preservatives, buffering agents and the like also may be added to the parenteral dosage forms. Particularly useful is the administration of a compound of any of the Formulas I-III directly in parenteral formulations. The pharmaceutical compositions are prepared by conventional techniques appropriate to the desired preparation containing appropriate amounts of the active ingredient, that is, the compound of any of the Formulas I-III according to the invention. See, for example, Remington: The Science and Practice of Pharmacy, 20th Edition. Baltimore, Md.: Lippincott Williams & Wilkins, 2000.

The dosage of the compounds of any of the Formulas I-V to achieve a therapeutic effect will depend not only on such factors as the age, weight and sex of the patient and mode of administration, but also on the degree of potassium channel activating activity desired and the potency of the particular compound being utilized for the particular disorder of disease concerned. It is also contemplated that the treatment and dosage of the particular compound may be administered in unit dosage form and that one skilled in the art would adjust the unit dosage form accordingly to reflect the relative level of activity. The decision as to the particular dosage to be employed (and the number of times to be administered per day is within the discretion of the physician, and may be varied by titration of the dosage to the particular circumstances of this invention to produce the desired therapeutic effect.

A suitable dose of a compound of any of the Formulas I-V or pharmaceutical composition thereof for a mammal, including man, suffering from, or likely to suffer from any condition as described herein is an amount of active ingredient from about 0.01 .mg/kg to 10 mg/kg body weight. For parenteral administration, the dose may be in the range of 0.1 .mg/kg to 1 mg/kg body weight for intravenous administration. For oral administration, the dose may be in the range about 0.1 .mg/kg to 5 mg/kg body weight. The active ingredient will preferably be administered in equal doses from one to four times a day. However, usually a small dosage is administered, and the dosage is gradually increased until the optimal dosage for the host under treatment is determined.

However, it will be understood that the amount of the compound actually administered will be determined by a physician, in the light of the relevant circumstances including the condition to be treated, the choice of compound of be administered, the chosen route of administration, the age, weight, and response of the individual patient, and the severity of the patient's symptoms.

The amount of the compound of the present invention or a pharmaceutically acceptable salt thereof that is effective for inhibiting mTOR or PI3K in a subject. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one compound of the present invention or a pharmaceutically acceptable salt thereof is administered, the effective dosage amounts correspond to the total amount administered.

In one embodiment, the compound of the present invention or a pharmaceutically acceptable salt thereof is administered concurrently with another therapeutic agent.

In one embodiment, a composition comprising an effective amount of a compound of the present invention or a pharmaceutically acceptable salt thereof and an effective amount of another therapeutic agent within the same composition can be administered.

Effective amounts of the other therapeutic agents are well known to those skilled in the art. However, it is well within the skilled artisan's purview to determine the other therapeutic agent's optimal effective amount range. The compound of the present invention or a pharmaceutically acceptable salt thereof and the other therapeutic agent can act additively or, in one embodiment, synergistically. In one embodiment, of the invention, where another therapeutic agent is administered to an animal, the effective amount of the compound of the present invention or a pharmaceutically acceptable salt thereof is less than its effective amount would be where the other therapeutic agent is not administered. In this case, without being bound by theory, it is believed that the compound of the present invention or a pharmaceutically acceptable salt thereof and the other therapeutic agent act synergistically.

Procedures used to synthesize the compounds of the present invention are described in Schemes 1-6 and are illustrated in the examples. Reasonable variations of the described procedures, which would be evident to one skilled in the art, are intended to be within the scope of the present invention:

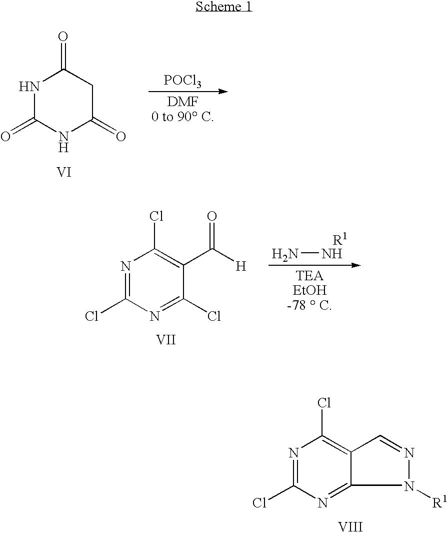

The key dichloro intermediate VIII may be prepared according to Scheme 1 by first reacting barbituric acid VI with POCl₃ in DMF then subjecting the chloroaldehyde VI to treatment with hydrazines H₂N—NH—R¹. The hydrazines can be purchased commercially or prepared synthetically via standard organic chemistry protocols.

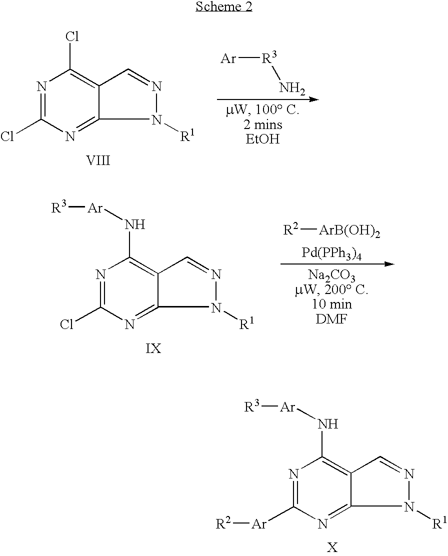

The synthesis of the 4-amino-6-aryl-1H-pyrazolo[3,4-d]pyrimidine compounds X may be accomplished according to Scheme 2 by first reacting the dichloride intermediate VIII with various amines under microwave conditions then subjecting the resulting 6-chloro-1H-pyrazolo[3,4-d]pyrimidine to Suzuki reaction with boronic acids also under microwave conditions.

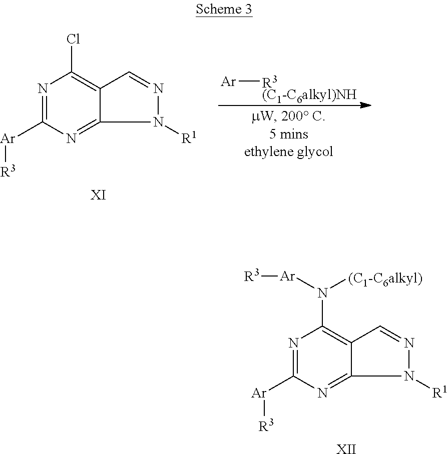

The 4-anilino-6-aryl-1H-pyrazolo[3,4-d]pyrimidine compounds XII may be prepared according to Scheme 3 by reacting the chloride intermediate XI with various disubstituted anilines under microwave conditions.

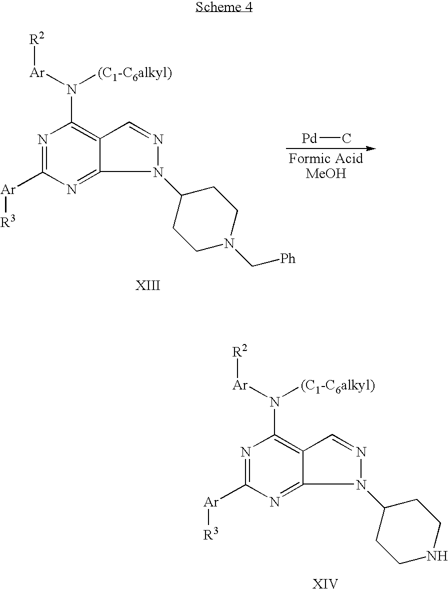

The 1-piperidinyl-4-amino-6-aryl-1H-pyrazolo[3,4-d]pyrimidine compounds XIV may be prepared according to Scheme 4 reacting the benzyl intermediate XIII with Pd—C under standard transfer hydrogenation conditions.

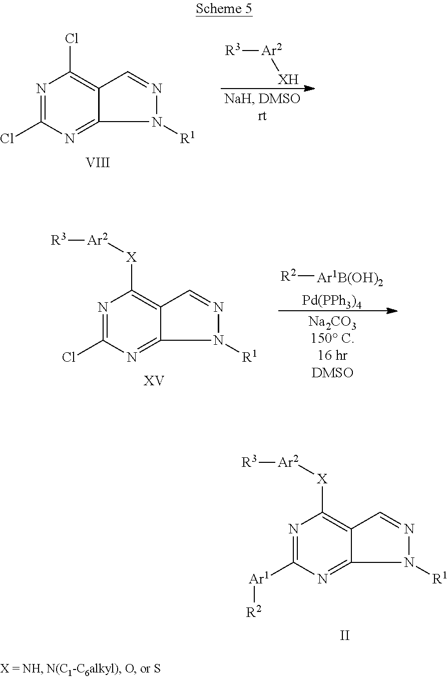

The 6-aryl-1H-pyrazolo[3,4-d]pyrimidine compounds II may be prepared according to Scheme 5 by first reacting the 4,6-dichloro-1H-pyrazolo[3,4-d]pyrimidine intermediate VIII with various phenols, heteroaryl phenols, arylmercaptans, heteroarylmercaptans, heteroarylamines, or anilines then subjecting the resulting 6-chloro-1H-pyrazolo[3,4-d]pyrimidine compounds XV to Suzuki reaction with the appropriate boronic acid.

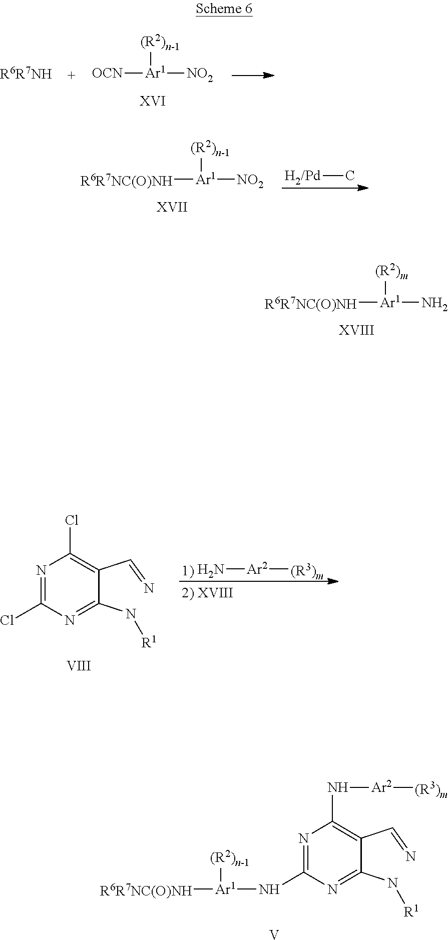

The 6-amino-1H-pyrazolo[3,4-d]pyrimidine compounds V (Formula I with X═NH, q=1, n>0, and one of R²═R⁶R⁷NC(O)NH—) may be prepared according to Scheme 6 by first reacting amine R⁶R⁷NH with isocyanate XVI to give the urea XVII, which is smoothly reduced to the amine. The dichloro intermediate VIII is selectively reacted with amine H₂NAr²(R³)ₘ at the 4-position of the 1H-pyrazolo[3,4-d]pyrimidine ring followed by reaction at the 6-position by amine XVIII. Various phenols, heteroaryl phenols, arylmercaptans, and heteroarylmercaptans could be used in place of amine XVIII to make other compounds of Formula I with q=1.

One of skill in the art will recognize that Schemes 1-6 can be adapted to produce the other compounds of Formulas I-V and pharmaceutically acceptable salts of compounds of Formulas I-V according to the present invention.

### EXAMPLES

The following abbreviations are used herein and have the indicated definitions: ACN is acetonitrile, ATP is adenosine triphosphate, Celite™ is flux-calcined diatomaceous earth. Celite™ is a registered trademark of World Minerals Inc. CHAPS is (3-[(3-cholamidopropyl)dimethyl ammonio]-1-propanesulfonic acid, DMF is N,N-dimethylformamide, and DMSO is dimethylsulfoxide. EDTA is ethylenediaminetetraacetic acid, EtOAc is ethyl acetate, and EtOH is ethanol. HEPES is 4-(2-hydroxyethyl)-1-piperazineethanesulfonic acid, GMF is glass microfiber, and HPLC is high-pressure liquid chromatography. Magnesol™ is a hydrated, synthetic, amorphous magnesium silicate. Magnesol™ is a registered trademark of the Dallas Group of America Inc. MeOH is methanol, MS is mass spectrometry, and PBS is phosphate-buffered saline (pH 7.4). TEA is NEt₃ or triethylamine, TFA is trifluoroacetic acid, THF is tetrahydrofuran, and TRIS is tris(hydroxymethyl)aminomethane.

### Synthetic Methods

The following methods outline the synthesis of the Examples in Table 1 of the present invention.

### Experimental for the Preparation of 4,6-Dichloro-1-methyl-1H-pyrazolo[3,4-d]pyrimidine (Scheme 1)

To a solution of POCl₃ (200 mL) in DMF (42 mL) cooled to 0° C. was slowly added barbituric acid (30 g) over 1.5 hrs. The mixture was then heated to reflux for 16 hrs and then removed from heating. The clear brown solution was added very slowly to a solution of ice water upon which a beige solid formed. The solid was filtered, dissolved in dichloromethane, washed with water, washed with a sat NaHCO₃ solution, dried (Na₂SO₄), and concentrated in vacuo to afford the desired chloroaldehyde.

To a solution of the chloroaldehyde (3.7 g, 17.5 mmol) dissolved in EtOH (50 mL) cooled to −78° C. was added methyl hydrazine (0.93 mL, 17.5 mmol) and TEA (8 mL). The mixture was stirred for 30 minutes at −78° C. then 2 hr at 0° C. The solution was concentrated in vacuo without heating. To the reduced volume solution EtOAc was added and the solution washed with a sat NaHCO₃ solution and concentrated in vacuo without heating. Filtration over a small silica gel plug (2:1 EtOAc:Hex) and concentration afforded the desired product as a yellow solid.

### Experimental for the Preparation of 4,6-Dichloro-1-(1-ethyl-piperidin-4-yl)-1H-pyrazolo[3,4-d]pyrimidine Compounds (Scheme 1)

To a solution of POCl₃ (200 mL) in DMF (42 mL) cooled to 0° C. was slowly added barbituric acid (30 g) over 1.5 hrs. The mixture was then heated to reflux for 16 hrs and then removed from heating. The clear brown solution was added very slowly to a solution of ice water upon which a beige solid formed. The solid was filtered, dissolved in DCM, washed with water, washed with a sat NaHCO₃ solution, dried (Na₂SO₄), and concentrated in vacuo to afford the desired chloroaldehyde.

To a solution of the chloroaldehyde (2.5 g, 11.6 mmol) dissolved in EtOH (40 mL) cooled to −78° C. was added N-benzyl-4-piperidinzyl-hydrazine (3.3 g, 11.6 mmol) and TEA (5 mL). The mixture was stirred for 30 minutes at −78° C. then 2 hr at 0° C. The solution was concentrated in vacuo without heating. To the reduced volume solution EtOAc and a sat NaHCO₃ solution was added and the solution filtered over Celite® and separated. The organic layer was dried (Na₂SO₄) and concentrated in vacuo without heating. Filtration over a small silica gel plug (EtOAc) and concentration afforded the desired product as a yellow solid.

### Experimental for the Preparation of (1-Substituted-6-Aryl-1H-pyrazolo[3,4-d]pyrimidin-4-yl)-Aryl-amine (Scheme 2)

To a solution of the dichloride (0.56 g, 3.31 mmol) dissolved in EtOH (4 mL) in a microwave conical vial was added the desired aniline (1 eq). The reaction heated under μW irradiation at 100 C for 2 minutes. The crude reaction then concentrated and the crude product used in the next step. The portion of the product used (0.76 mmol) was dissolved in DMF (3 mL) and added with the desired aryl boronic acid (1.14 mmol, 1.5 eq), Na₂CO₃ solution (0.76 mL, 2M, 1.6 mmol), and Pd(PPh₃)₄ (10 mgs) to the microwave conical vial. The reaction heated under μW irradiation at 200° C. for 10 minutes. The crude reaction then concentrated and purified via preparatory HPLC using a Gilson instrument.

### Experimental for the Preparation of (1-Substituted-6-Aryl-1H-pyrazolo[3,4-d]pyrimidin-4-yl)-Aryl-Substituted amine (Scheme 3)

To a solution of the chloride (30 mg, 0.122 mmol) dissolved in ethylene glycol (1 mL) added the desired N-substituted aniline (3 eq) in a microwave conical vial. The reaction heated under μW irradiation at 200 C for 5 minutes. The crude reaction then concentrated and purified via preparatory HPLC using a Gilson instrument.

### Experimental for the Preparation of 4-Substituted-4-yl-6-aryl-1-piperidin-4-yl-1H-pyrazolo[3,4-d]pyrimidine (Scheme 4)

To a solution of the benzyl piperidinyl substrate (25-50 mgs) dissolved in a MeOH/4% formic acid solution (2 mL) was added Pd/C (50 mgs). The mixture was stirred for 15 hrs filtered, concentrated and purified via preparatory HPLC using a Gilson instrument.

### Experimental for the Preparation of 1-Substituted-4-phenoxy-6-aryl-1H-pyrazolo[3,4-d]pyrimidine (Scheme 5)

To a solution of the dichloride (2.53 mmol) dissolved in DMSO (5 mL) was added by drops a solution of phenol (0.7 eq) and NaH (0.7 eq) that had stirred for 30 minutes. The reaction mixture was stirred for 5 hr at room temperature and then used as a DMSO solution for the next step. To the portion of the product used (0.25 mmol) was added with the desired aryl boronic acid (1.5 eq), Na₂CO₃ solution (2 eq), and Pd(PPh₃)₄ (catalytic amount). The reaction heated at 150° C. for 16 hrs. The crude reaction then filtered purified via preparatory HPLC using a Gilson instrument.

Preparatory HPLC Purification Procedure using a Gilson Instrument

The Gilson crude material was dissolved in 1.5 ml DMSO: 0.5 ml acetonitrile, filtered through a 0.45 μm GMF, and purified on a Gilson HPLC, using a Phenomenex LUNA C₁₈ column: 60 mm×21.20 mm I.D., 5 μm particle size: with ACN/water (containing 0.2% TFA or Et₃N) gradient elution. The appropriate fractions were analyzed by LC/MS as described below. Combining pure fractions and evaporating the solvent in a Speed-Vac isolated the title compound.

HPLC: Analytical Method and Parameters:

Instrument: HP Agilent 1100 LC/MS; UV Detector: Agilent 1100 Diode Array Detector; Mass Spectrometer Detector: Agilent MSD; Column: Waters Xterra MS C18 30 mm×2.1 mm i.d., 3.5 μm; Flow Rate: 1.00 ml/min; Run Time: 5.00 min; Gradient Elution: 0 min 90% water, 10% acetonitrile; 3 min 10% water, 90% acetonitrile; Column Temperature: 50° C.; UV Signals: 215 nm, 254 nm; MS Parameters: Mass Range 100-1000, Fragmentor 140, Gain EMV 1.0.

The 6-aryl-1H-pyrazolo[3,4-d]pyrimidine compounds in Table 1 were prepared according to the above procedures.

TABLE 1

| | | LC/MS | | | | |
|---|---|---|---|---|---|---|
| | | MW | Mol | Retention | HPLC | Synthetic |
| Example | Name | Observed | Ion | Time | Conditions | Method |
| 1 | 4-{[1-(1-benzylpiperidin-4-yl)- | 504.2 | M + H | 2.07 | std method | 2 |
| | 6-phenyl-1H-pyrazolo[3,4- | | | | w/formic | |
| | d]pyrimidin-4- | | | | | |
| | yl]amino}benzamide | | | | | |
| 2 | 1-(1-benzylpiperidin-4-yl)-N- | 521.3 | M + H | 2.24 | std method | 2 |
| | (3,4-dimethoxyphenyl)-6- | | | | w/formic | |
| | phenyl-1H-pyrazolo[3,4- | | | | | |
| | d]pyrimidin-4-amine | | | | | |
| 3 | 3-{[1-(1-benzylpiperidin-4-yl)- | 504.2 | M + H | 2.07 | std method | 2 |
| | 6-phenyl-1H-pyrazolo[3,4- | | | | w/formic | |
| | d]pyrimidin-4- | | | | | |
| | yl]amino}benzamide | | | | | |
| 4 | 1-(1-benzylpiperidin-4-yl)-N- | 500.2 | M + H | 2.25 | std method | 2 |
| | 1H-indol-5-yl-6-phenyl-1H- | | | | w/formic | |
| | pyrazolo[3,4-d]pyrimidin-4- | | | | | |
| | amine | | | | | |
| 5 | N-(3-{[1-(1-benzylpiperidin-4- | 518.3 | M + H | 2.16 | std method | 2 |
| | yl)-6-phenyl-1H-pyrazolo[3,4- | | | | w/formic | |
| | d]pyrimidin-4- | | | | | |
| | yl]amino}phenyl)acetamide | | | | | |
| 6 | 4-{[1-(1-benzylpiperidin-4-yl)- | 520.2 | M + H | 1.94 | std method | 2 |
| | 6-(3-hydroxyphenyl)-1H- | | | | w/formic | |
| | pyrazolo[3,4-d]pyrimidin-4- | | | | | |
| | yl]amino}benzamide | | | | | |
| 7 | 3-{1-(1-benzylpiperidin-4-yl)- | 537.3 | M + H | 2.08 | std method | 2 |
| | 4-[(3,4- | | | | w/formic | |
| | dimethoxyphenyl)amino]-1H- | | | | | |
| | pyrazolo[3,4-d]pyrimidin-6- | | | | | |
| | yl}phenol | | | | | |
| 8 | 3-{[1-(1-benzylpiperidin-4-yl)- | 520.2 | M + H | 1.96 | std method | 2 |
| | 6-(3-hydroxyphenyl)-1H- | | | | w/formic | |
| | pyrazolo[3,4-d]pyrimidin-4- | | | | | |
| | yl]amino}benzamide | | | | | |
| 9 | 3-[1-(1-benzylpiperidin-4-yl)- | 516.2 | M + H | 2.07 | std method | 2 |
| | 4-(1H-indol-5-ylamino)-1H- | | | | w/formic | |
| | pyrazolo[3,4-d]pyrimidin-6- | | | | | |
| | yl]phenol | | | | | |
| 10 | N-(3-{[1-(1-benzylpiperidin-4- | 534.3 | M + H | 2.05 | std method | 2 |
| | yl)-6-(3-hydroxyphenyl)-1H- | | | | w/formic | |
| | pyrazolo[3,4-d]pyrimidin-4- | | | | | |
| | yl]amino}phenyl)acetamide | | | | | |
| 11 | 4-[(1-methyl-6-phenyl-1H- | 343.1 | M − H | 2.25 | std method | 2 |
| | pyrazolo[3,4-d]pyrimidin-4- | | | | w/ NH4OAc | |
| | yl)amino]benzamide | | | | | |
| 12 | 4-[(6-phenyl-1-piperidin-4-yl- | 414.2 | M + H | 1.78 | std method | 4 |
| | 1H-pyrazolo[3,4-d]pyrimidin- | | | | w/formic | |
| | 4-yl)amino]benzamide | | | | | |
| 13 | N-(3,4-dimethoxyphenyl)-6- | 431.2 | M + H | 1.94 | std method | 4 |
| | phenyl-1-piperidin-4-yl-1H- | | | | w/formic | |
| | pyrazolo[3,4-d]pyrimidin-4- | | | | | |
| | amine | | | | | |
| 14 | 3-[(6-phenyl-1-piperidin-4-yl- | 414.2 | M + H | 1.8 | std method | 4 |
| | 1H-pyrazolo[3,4-d]pyrimidin- | | | | w/formic | |
| | 4-yl)amino]benzamide | | | | | |
| 15 | N-{3-[(6-phenyl-1-piperidin-4- | 428.2 | M + H | 1.88 | std method | 4 |
| | yl-1H-pyrazolo[3,4- | | | | w/formic | |
| | d]pyrimidin-4- | | | | | |
| | yl)amino]phenyl}acetamide | | | | | |
| 16 | 4-{[6-(3-hydroxyphenyl)-1- | 430.2 | M + H | 1.69 | std method | 4 |
| | piperidin-4-yl-1H- | | | | w/formic | |
| | pyrazolo[3,4-d]pyrimidin-4- | | | | | |
| | yl]amino}benzamide | | | | | |
| 17 | 3-{4[(3,4- | 447.2 | M + H | 1.84 | std method | 4 |
| | dimethoxyphenyl)amino]-1- | | | | w/formic | |
| | piperidin-4-yl-1H- | | | | | |
| | pyrazolo[3,4-d]pyrimidin-6- | | | | | |
| | yl}phenol | | | | | |
| 18 | 3-{[6-(3-hydroxyphenyl)-1- | 430.2 | M + H | 1.7 | std method | 4 |
| | piperidin-4-yl-1H- | | | | w/formic | |
| | pyrazolo[3,4-d]pyrimidin-4- | | | | | |
| | yl]amino}benzamide | | | | | |
| 19 | 3-[4-(1H-indol-5-ylamino)-1- | 426.2 | M + H | 1.83 | std method | 4 |
| | piperidin-4-yl-1H- | | | | w/formic | |
| | pyrazolo[3,4-d]pyrimidin-6- | | | | | |
| | yl]phenol | | | | | |
| 20 | N-(3-{[6-(3-hydroxyphenyl)-1- | 444.2 | M + H | 1.8 | std method | 4 |
| | piperidin-4-yl-1H- | | | | w/formic | |
| | pyrazolo[3,4-d]pyrimidin-4- | | | | | |
| | yl]amino}phenyl)acetamide | | | | | |
| 21 | 4-{[1-(1-benzylpiperidin-4-yl)- | 549.2 | M + H | 2.08 | std method | 2 |
| | 6-(3-nitrophenyl)-1H- | | | | w/formic | |
| | pyrazolo[3,4-d]pyrimidin-4- | | | | | |
| | yl]amino}benzamide | | | | | |
| 22 | 4-({1-(1-benzylpiperidin-4-yl)- | 534.3 | M + H | 1.87 | std method | 2 |
| | 6-[4-(hydroxymethyl)phenyl]- | | | | w/formic | |
| | 1H-pyrazolo[3,4-d]pyrimidin- | | | | | |
| | 4-yl}amino)benzamide | | | | | |
| 23 | 4-{[1-(1-benzylpiperidin-4-yl)- | 534.3 | M + H | 2 | std method | 2 |
| | 6-(3-methoxyphenyl)-1H- | | | | w/formic | |
| | pyrazolo[3,4-d]pyrimidin-4- | | | | | |
| | yl]amino}benzamide | | | | | |
| 24 | 4-{[1-(1-benzylpiperidin-4-yl)- | 520.2 | M + H | 1.85 | std method | 2 |
| | 6-(4-hydroxyphenyl)-1H- | | | | w/formic | |
| | pyrazolo[3,4-d]pyrimidin-4- | | | | | |
| | yl]amino}benzamide | | | | | |
| 25 | 4-({1-(1-benzylpiperidin-4-yl)- | 572.2 | M + H | 2.17 | std method | 2 |
| | 6-[3-(trifluoromethyl)phenyl]- | | | | w/formic | |
| | 1H-pyrazolo[3,4-d]pyrimidin- | | | | | |
| | 4-yl}amino)benzamide | | | | | |
| 26 | 4-{[1-(1-benzylpiperidin-4-yl)- | 520.2 | M + H | 1.97 | std method | 2 |
| | 6-(2-hydroxyphenyl)-1H- | | | | w/formic | |
| | pyrazolo[3,4-d]pyrimidin-4- | | | | | |
| | yl]amino}benzamide | | | | | |
| 27 | 1-(1-benzylpiperidin-4-yl)-N- | 566.2 | M + H | 2.24 | std method | 2 |
| | (3,4-dimethoxyphenyl)-6-(3- | | | | w/formic | |
| | nitrophenyl)-1H-pyrazolo[3,4- | | | | | |
| | d]pyrimidin-4-amine | | | | | |
| 28 | (4-{1-(1-benzylpiperidin-4-yl)- | 551.3 | M + H | 1.99 | std method | 2 |
| | 4-[(3,4- | | | | w/formic | |
| | dimethoxyphenyl)amino]-1H- | | | | | |
| | pyrazolo[3,4-d]pyrimidin-6- | | | | | |
| | yl}phenyl)methanol | | | | | |
| 29 | 1-(1-benzylpiperidin-4-yl)-N- | 551.3 | M + H | 2.21 | std method | 2 |
| | (3,4-dimethoxyphenyl)-6-(3- | | | | w/formic | |
| | methoxyphenyl)-1H- | | | | | |
| | pyrazolo[3,4-d]pyrimidin-4- | | | | | |
| | amine | | | | | |
| 30 | 4-{1-(1-benzylpiperidin-4-yl)- | 537.3 | M + H | 1.99 | std method | 2 |
| | 4-[(3,4- | | | | w/formic | |
| | dimethoxyphenyl)amino]-1H- | | | | | |
| | pyrazolo[3,4-d]pyrimidin-6- | | | | | |
| | yl}phenol | | | | | |
| 31 | 1-(1-benzylpiperidin-4-yl)-N- | 589.2 | M + H | 2.32 | std method | 2 |
| | (3,4-dimethoxyphenyl)-6-[3- | | | | w/formic | |
| | (trifluoromethyl)phenyl]-1H- | | | | | |
| | pyrazolo[3,4-d]pyrimidin-4- | | | | | |
| | amine | | | | | |
| 32 | 2-{1-(1-benzylpiperidin-4-yl)- | 537.3 | M + H | 2.13 | std method | 2 |
| | 4-[(3,4- | | | | w/formic | |
| | dimethoxyphenyl)amino]-1H- | | | | | |
| | pyrazolo[3,4-d]pyrimidin-6- | | | | | |
| | yl}phenol | | | | | |
| 33 | N,1-dimethyl-N,6-diphenyl- | 316.1 | M + H | 2.94 | std method | 3 |
| | 1H-pyrazolo[3,4-d]pyrimidin- | | | | w/formic | |
| | 4-amine | | | | | |
| 34 | 4-[methyl(1-methyl-6-phenyl- | 359.2 | M + H | 2.46 | std method | 3 |
| | 1H-pyrazolo[3,4-d]pyrimidin- | | | | w/formic | |
| | 4-yl)amino]benzamide | | | | | |
| 35 | N-{4-[methyl(1-methyl-6- | 373.2 | M + H | 2.59 | std method | 3 |
| | phenyl-1H-pyrazolo[3,4- | | | | w/formic | |
| | d]pyrimidin-4- | | | | | |
| | yl)amino]phenyl}acetamide | | | | | |
| 36 | N-(3,4-dimethoxyphenyl)-N,1- | 376.2 | M + H | 2.81 | std method | 3 |
| | dimethyl-6-phenyl-1H- | | | | w/formic | |
| | pyrazolo[3,4-d]pyrimidin-4- | | | | | |
| | amine | | | | | |
| 37 | N-(4-methoxyphenyl)-N,1- | 346.2 | M + H | 2.91 | std method | 3 |
| | dimethyl-6-phenyl-1H- | | | | w/formic | |
| | pyrazolo[3,4-d]pyrimidin-4- | | | | | |
| | amine | | | | | |
| 38 | 4-(1H-indol-5-yloxy)-6-(3- | 372.1 | M + H | 2.61 | std method | 5 |
| | methoxyphenyl)-1-methyl-1H- | | | | w/formic | |
| | pyrazolo[3,4-d]pyrimidine | | | | | |
| 39 | 6-(3,4-dimethoxyphenyl)-4- | 402.1 | M + H | 2.53 | std method | 5 |
| | (1H-indol-5-yloxy)-1-methyl- | | | | w/formic | |
| | 1H-pyrazolo[3,4-d]pyrimidine | | | | | |
| 40 | 4-[4-(1H-indol-5-yloxy)-1- | 385.2 | M + H | 2.64 | std method | 5 |
| | methyl-1H-pyrazolo[3,4- | | | | w/formic | |
| | d]pyrimidin-6-yl]-N,N- | | | | | |
| | dimethylaniline | | | | | |
| 41 | 4-(1H-indol-5-yloxy)-1- | 343.1 | M + H | 2.25 | std method | 5 |
| | methyl-6-pyridin-3-yl-1H- | | | | w/formic | |
| | pyrazolo[3,4-d]pyrimidine | | | | | |
| 42 | 4-(1H-indol-5-yloxy)-1- | 410.1 | M + H | 2.77 | std method | 5 |
| | methyl-6-[3- | | | | w/formic | |
| | (trifluoromethyl)phenyl]-1H- | | | | | |
| | pyrazolo[3,4-d]pyrimidine | | | | | |
| 43 | 3-[4-(1H-indol-5-yloxy)-1- | 367.1 | M + H | 2.61 | std method | 5 |
| | methyl-1H-pyrazolo[3,4- | | | | w/formic | |
| | d]pyrimidin-6-yl]benzonitrile | | | | | |
| 44 | 6-biphenyl-3-yl-4-(1H-indol-5- | 418.2 | M + H | 2.88 | std method | 5 |
| | yloxy)-1-methyl-1H- | | | | w/formic | |
| | pyrazolo[3,4-d]pyrimidine | | | | | |
| 45 | 4-(3,4-dimethoxyphenoxy)-6- | 393.1 | M + H | 2.6 | std method | 5 |
| | (3-methoxyphenyl)-1-methyl- | | | | w/formic | |
| | 1H-pyrazolo[3,4-d]pyrimidine | | | | | |
| 46 | 4-(3,4-dimethoxyphenoxy)-6- | 423.2 | M + H | 2.52 | std method | 5 |
| | (3,4-dimethoxyphenyl)-1- | | | | w/formic | |
| | methyl-1H-pyrazolo[3,4- | | | | | |
| | d]pyrimidine | | | | | |
| 47 | 4-[4-(3,4-dimethoxyphenoxy)- | 406.2 | M + H | 2.65 | std method | 5 |
| | 1-methyl-1H-pyrazolo[3,4- | | | | w/formic | |
| | d]pyrimidin-6-yl]-N,N- | | | | | |
| | dimethylaniline | | | | | |
| 48 | 6-biphenyl-3-yl-4-(3,4- | 439.2 | M + H | 2.89 | std method | 5 |
| | dimethoxyphenoxy)-1-methyl- | | | | w/formic | |
| | 1H-pyrazolo[3,4-d]pyrimidine | | | | | |
| 49 | N-(4-{[6-(3-methoxyphenyl)- | 390.1 | M + H | 2.45 | std method | 5 |
| | 1-methyl-1H-pyrazolo[3,4- | | | | w/formic | |
| | d]pyrimidin-4- | | | | | |
| | yl]oxy}phenyl)acetamide | | | | | |
| 50 | N-(4-{[6-(3-hydroxyphenyl)-1- | 376.1 | M + H | 2.17 | std method | 5 |
| | methyl-1H-pyrazolo[3,4- | | | | w/formic | |
| | d]pyrimidin-4- | | | | | |
| | yl]oxy}phenyl)acetamide | | | | | |
| 51 | N-[4-({6-[4- | 403.2 | M + H | 2.48 | std method | 5 |
| | (dimethylamino)phenyl]-1- | | | | w/formic | |
| | methyl-1H-pyrazolo[3,4- | | | | | |
| | d]pyrimidin-4- | | | | | |
| | yl}oxy)phenyl]acetamide | | | | | |
| 52 | N-[4-({1-methyl-6-[3- | 428.1 | M + H | 2.63 | std method | 5 |
| | (trifluoromethyl)phenyl]-1H- | | | | w/formic | |
| | pyrazolo[3,4-d]pyrimidin-4- | | | | | |
| | yl}oxy)phenyl]acetamide | | | | | |
| 53 | N-{4-[(6-biphenyl-3-yl-1- | | | | | 5 |
| | methyl-1H-pyrazolo[3,4- | | | | | |
| | d]pyrimidin-4- | | | | | |
| | yl)oxy]phenyl}acetamide | | | | | |
| 54 | N-(4-{[6-(4-hydroxyphenyl)-1- | 376.1 | M + H | 2.17 | std method | 5 |
| | methyl-1H-pyrazolo[3,4- | | | | w/formic | |
| | d]pyrimidin-4- | | | | | |
| | yl]oxy}phenyl)acetamide | | | | | |
| 55 | N-(4-{[6-(1H-indol-5-yl)-1- | | | | | 5 |
| | methyl-1H-pyrazolo[3,4- | | | | | |
| | d]pyrimidin-4- | | | | | |
| | yl]oxy}phenyl)acetamide | | | | | |
| 56 | 6-biphenyl-4-yl-4-(1H-indol-5- | 418.2 | M + H | 2.83 | std method | 5 |
| | yloxy)-1-methyl-1H- | | | | w/formic | |
| | pyrazolo[3,4-d]pyrimidine | | | | | |
| 57 | 4-(1H-indol-5-yloxy)-6-(6- | 422.2 | M + H | 2.72 | std method | 5 |
| | methoxy-2-naphthyl)-1- | | | | w/formic | |
| | methyl-1H-pyrazolo[3,4- | | | | | |
| | d]pyrimidine | | | | | |
| 58 | 4-(1H-indol-5-yloxy)-6-(4- | 384.2 | M + H | 2.77 | std method | 5 |
| | isopropylphenyl)-1-methyl- | | | | w/formic | |
| | 1H-pyrazolo[3,4-d]pyrimidine | | | | | |
| 59 | 4-(1H-indol-5-yloxy)-1- | 346.1 | M + H | 2.21 | std method | 5 |
| | methyl-6-(1-methyl-1H- | | | | w/formic | |
| | pyrazol-4-yl)-1H-pyrazolo[3,4- | | | | | |
| | d]pyrimidine | | | | | |
| 60 | 4-[(6-biphenyl-4-yl-1-methyl- | 421.2 | M + H | 2.6 | std method | 2 |
| | 1H-pyrazolo[3,4-d]pyrimidin- | | | | w/formic | |
| | 4-yl)amino]benzamide | | | | | |
| 61 | 4-{[6-(4-isopropylphenyl)-1- | 387.2 | M + H | 2.5 | std method | 2 |
| | methyl-1H-pyrazolo[3,4- | | | | w/formic | |
| | d]pyrimidin-4- | | | | | |
| | yl]amino}benzamide | | | | | |
| 62 | 4-{[1-methyl-6-(1-methyl-1H- | 349.1 | M + H | 1.92 | std method | 2 |
| | pyrazol-4-yl)-1H-pyrazolo[3,4- | | | | w/formic | |
| | d]pyrimidin-4- | | | | | |
| | yl]amino}benzamide | | | | | |
| 63 | 4-{[6-(3,5-dimethylisoxazol-4- | 362.1 | M − H | 2.07 | std method | 2 |
| | yl)-1-methyl-1H-pyrazolo[3,4- | | | | w/NH4OAc | |
| | d]pyrimidin-4- | | | | | |
| | yl]amino}benzamide | | | | | |
| 64 | 4-{[6-(3-chlorophenyl)-1- | 379.1 | M + H | 2.43 | std method | 2 |
| | methyl-1H-pyrazolo[3,4- | | | | w/formic | |
| | d]pyrimidin-4- | | | | | |
| | yl]amino}benzamide | | | | | |
| 65 | 4-{[6-(3-furyl)-1-methyl-1H- | 333.1 | M − H | 2.04 | std method | 2 |
| | pyrazolo[3,4-d]pyrimidin-4- | | | | w/NH4OAc | |
| | yl]amino}benzamide | | | | | |
| 66 | 6-(3-chlorophenyl)-4-(1H- | 376.1 | M + H | 2.72 | std method | 5 |
| | indol-5-yloxy)-1-methyl-1H- | | | | w/formic | |
| | pyrazolo[3,4-d]pyrimidine | | | | | |
| 67 | 4-(1H-indol-5-yloxy)-1- | 385.1 | M − H | 2.63 | std method | 5 |
| | methyl-6-(3-nitrophenyl)-1H- | | | | w/NH4OAc | |
| | pyrazolo[3,4-d]pyrimidine | | | | | |
| 68 | 6-(3-furyl)-4-(1H-indol-5- | 332.1 | M + H | 2.34 | std method | 5 |
| | yloxy)-1-methyl-1H- | | | | w/formic | |
| | pyrazolo[3,4-d]pyrimidine | | | | | |
| 69 | 6-{3-[(2- | 482.1 | M + H | 2.82 | std method | 5 |
| | chlorobenzyl)oxy]phenyl}-4- | | | | w/formic | |
| | (1H-indol-5-yloxy)-1-methyl- | | | | | |
| | 1H-pyrazolo[3,4-d]pyrimidine | | | | | |
| 70 | 6-{4-[(3,5- | 508.2 | M + H | 2.72 | std method | 5 |
| | dimethoxybenzyl)oxy]phenyl}- | | | | w/formic | |
| | 4-(1H-indol-5-yloxy)-1- | | | | | |
| | methyl-1H-pyrazolo[3,4- | | | | | |
| | d]pyrimidine | | | | | |
| 71 | 6-{3-[(3,5- | 508.2 | M + H | 2.71 | std method | 5 |
| | dimethoxybenzyl)oxy]phenyl}- | | | | w/formic | |
| | 4-(1H-indol-5-yloxy)-1- | | | | | |
| | methyl-1H-pyrazolo[3,4- | | | | | |
| | d]pyrimidine | | | | | |
| 72 | 6-(3,5-dimethylisoxazol-4-yl)- | 361.1 | M + H | 2.45 | std method | 5 |
| | 4-(1H-indol-5-yloxy)-1- | | | | w/formic | |
| | methyl-1H-pyrazolo[3,4- | | | | | |
| | d]pyrimidine | | | | | |
| 73 | 4-(3,4-dimethoxyphenoxy)-1- | 363.1 | M + H | 2.77 | std method | 5 |
| | methyl-6-phenyl-1H- | | | | w/formic | |
| | pyrazolo[3,4-d]pyrimidine | | | | | |
| 74 | N-{4-[(1-methyl-6-phenyl-1H- | | | | | 5 |
| | pyrazolo[3,4-d]pyrimidin-4- | | | | | |
| | yl)oxy]phenyl}acetamide | | | | | |
| 75 | 4-[(1-methyl-6-phenyl-1H- | 346.1 | M + H | 2.49 | std method | 5 |
| | pyrazolo[3,4-d]pyrimidin-4- | | | | w/formic | |
| | yl)oxy]benzamide | | | | | |
| 76 | 4-(1H-indol-5-yloxy)-1- | 342.1 | M + H | 2.71 | std method | 5 |
| | methyl-6-phenyl-1H- | | | | w/formic | |
| | pyrazolo[3,4-d]pyrimidine | | | | | |

^{a}HPLC Conditions: Instrument - Agilent 1100; Column: Thermo Aquasil C18, 50 × 2.1 mm, 5 μm;
Mobile Phase A: 0.1% Formic Acid in water; B: 0.1% Formic Acid in CAN; Flow Rate: 0.800 mL/min;
Column Temperature: 40° C.; Injection Volume: 5 μL; UV: monitor 215, 230, 254, 280, and
300 nm; Purity is reported at 254 nm unless otherwise noted.
Gradient Table:

| | |
|---|---|
| Time (min) | % B |
| 0 | 5 |
| 2.5 | 95 |
| 4.0 | 95 |
| 4.1 | 5 |
| 5.5 | 5 |

^{b}MS Conditions: Instrument: Agilent MSD; Ionization Mode: API-ES; Gas Temperature:
350° C.; Drying Gas: 11.0 L/min.; Nebulizer Pressure: 55 psig; Polarity: 50% positive,
50% negative; VCap: 3000 V (positive), 2500 V (negative); Fragmentor: 80 (positive), 120 (negative);
Mass Range: 100-1000 m/z; Threshold: 150; Step size: 0.15; Gain: 1; Peak width: 0.15 min.

### Preparation of 4-(1-methyl-6-(4-(3-methylureido)phenyl)-1H-pyrazolo[3,4-d]pyrimidin-4-ylamino)benzamide: (Example 77)

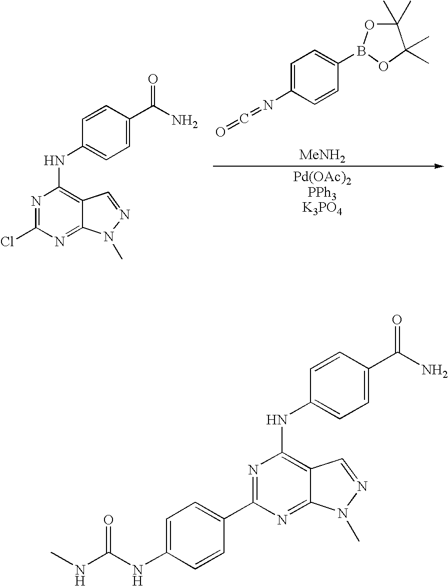

A solution of 4-isocyanatophenylboronic acid, pinacol ester (102 mg, 0.42 mmol), in dichloromethane (2 mL) was added to a solution of methylamine in THF (0.5 mL, 2.0 M). Resulting mixture sat for 5 minutes, then concentrated in vacuo. Residue dissolved in 1-methyl-2-pyrrolidinone (1 mL) and this solution added to a mixture of 4-(6-Chloro-1-methyl-1H-pyrazolo[3,4-d]pyrimidin-4-ylamino)benzamide (95 mg, 0.31 mmol), Pd(OAc)₂ (3 mg, 0.01 mmol), PPh₃ (10 mg, 0.04 mmol), and K₃PO₄ (203 mg, 0.96 mmol) in water (0.7 mL) in a microwave vial and then subjected to microwave conditions (165° C., 800 seconds), then (175° C., 30 minutes). The reaction mixture was cooled to room temperature. Saturated aqueous NaCl added (1 mL) and organic layer diluted with EtOH (3 mL) and resulting solution collected and concentrated. Crude material purified by preparative HPLC-MS (CH₃CN—H₂O-TFA) to give 4-(1-methyl-6-(4-(3-methylureido)phenyl)-1H-pyrazolo[3,4-d]pyrimidin-4-ylamino)benzamide, Yield: 21%. MS (m/z): 417.3 (M+1).

### Preparation of 1-methyl-3-(3-(4,4,5,5-tetramethyl-1,3,2-dioxaborolan-2-yl)phenyl)urea

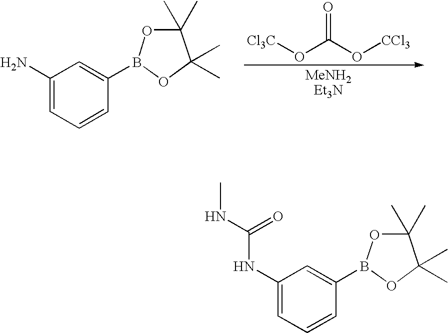

A solution of 3-(4,4,5,5-tetramethyl-1,3,2-dioxaborolan-2-yl)aniline (115 mg, 0.53 mmol) and triethylamine (0.146 ml, 1.05 mmol) in dichloromethane (2 mL) was added to a solution of triphosgene (51 mg, 0.17 mmol) in dichloromethane (2 mL) over a period of 30 seconds. Resulting mixture stirred for 5 minutes, then methylamine (0.525 ml, 1.05 mmol) added and this mixture stirred another 5 minutes. Reaction mixture concentrated and residue partitioned between EtOAc (5 mL) and H₂O (2 mL). Aqueous layer extracted with EtOAc (3×5 mL). EtOAc extracts combined, dried over Na₂SO₄, decanted, and concentrated. The product was used immediately.

### Preparation of 4-(1-methyl-6-(3-(3-methylureido)phenyl)-1H-pyrazolo[3,4-d]pyrimidin-4-ylamino)benzamide (Example 78)

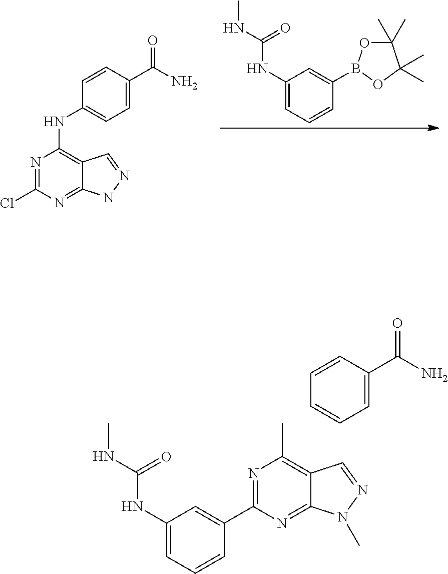

4-(6-Chloro-1-methyl-1H-pyrazolo[3,4-d]pyrimidin-4-ylamino)benzamide (121 mg, 0.40 mmol), 1-methyl-3-(3-(4,4,5,5-tetramethyl-1,3,2-dioxaborolan-2-yl)phenyl)urea (121 mg, 0.44 mmol), Pd(OAc)₂ (3.6 mg, 0.016 mmol), PPh₃ (12.6 mg, 0.048 mmol), and K₃PO₄ (255 mg, 1.20 mmol) were diluted with 1-methyl-2-pyrrolidinone (2.2 mL) and water (1 mL) in a microwave vial and subjected to microwave conditions (160° C., 5 minutes). The reaction mixture was cooled to room temperature. Saturated aqueous NaCl added (1 mL) and organic layer removed and diluted with MeOH (25 mL) and resulting solution filtered through Celite then the filtrate concentrated. The crude material was purified by preparative HPLC-MS (CH₃CN—H₂O—NH₄OH) to give 4-(1-methyl-6-(3-(3-methylureido)phenyl)-1H-pyrazolo[3,4-d]pyrimidin-4-ylamino)benzamide, Yield: 17%. MS (m/z): 417.2 (M+1).

### 4-{[1-Methyl-6-({4-[(pyridin-3-ylcarbamoyl)amino]phenyl}amino)-1H-pyrazolo[3,4-d]pyrimidin-4-yl]amino}benzamide (Example 79)

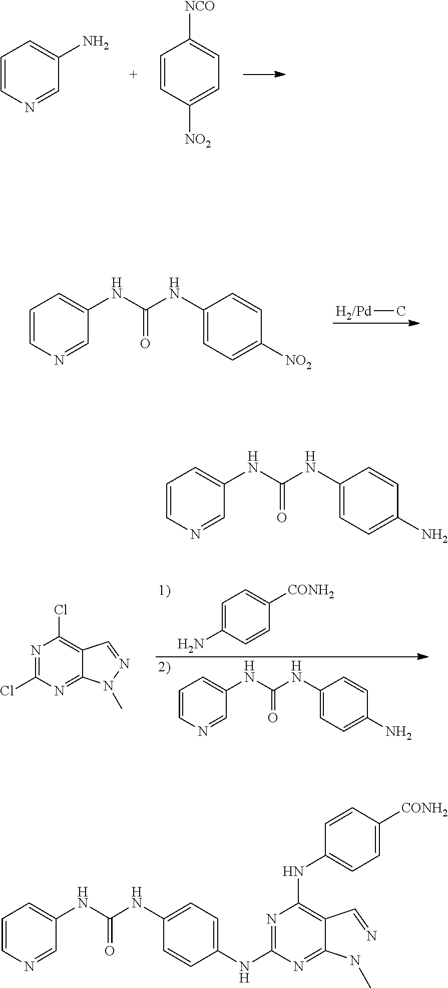

### Step A. 1-(3-Nitrophenyl)-3-pyridin-3-ylurea

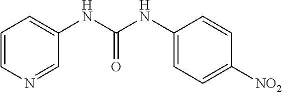

To a solution of 3-aminopyridine (564 mg, 6 mmol) in 30 mL of tetrahydrofuran was added 4-nitrophenyl isocyanate (998 mg, 6 mmol). The mixture was stirred at room temperature for 18 hours, and the product was filtered and dried to give 1.02 g (66%) of 1-(3-nitrophenyl)-3-pyridin-3-ylurea as a yellow solid. MS: m/z 259.1 (M+H).

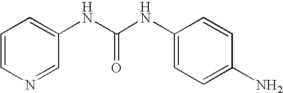

### Step B. 1-(3-Aminophenyl)-3-pyridin-3-ylurea

To a suspension of methyl 1-(3-nitrophenyl)-3-pyridin-3-ylurea (500 mg, 1.94 mmol) in 50 mL of methanol was added 200 mg 10% palladium on carbon. The mixture was hydrogenated using a −balloon for 2 hours, and filtered through Celite™. The filtrate was concentrated and the residue was chromatographed over silica gel, eluting with 5% methanol in ethyl acetate to give 261 mg (59%) of 1-(3-aminophenyl)-3-pyridin-3-ylurea as an off-white solid. MS: m/z 229.1 (M+H).

### Step C. 4-{[1-Methyl-6-({4-[(pyridin-3-ylcarbamoyl)amino]phenyl}amino)-1H-pyrazolo[3,4-d]pyrimidin-4-yl]amino}benzamide

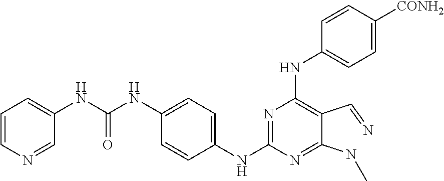

A mixture of 4,6-dichloro-1H-pyrazolo[3,4-d]pyrimidine (102 mg, 0.5 mmol) and 4-aminobenzamide (136 mg, 1.0 mmol) in 15 mL of ethanol was refluxed for 6 hours, and 1-(3-aminophenyl)-3-pyridin-3-ylurea (228 mg, 1.0 mmol) was added. The resulting mixture was refluxed for 3 days, and the solids formed were collected by filtration, and further purified by HPLC to give 28 mg (9.2%) of 4-{[1-methyl-6-({4-[(pyridin-3-ylcarbamoyl)amino]phenyl}amino)-1H-pyrazolo[3,4-d]pyrimidin-4-yl]amino}benzamide TFA salt as a tan solid. MS: m/z 495.1 (M+H).

The 6-amino-1H-pyrazolo[3,4-d]pyrimidine compounds in Table 2 were prepared according to the above procedures.

TABLE 2

| | | LC/MS | |
|---|---|---|---|
| | | MW | |
| Example | Name | Observed | Mol Ion |
| 80 | 4-{[1-Methyl-6-({3-[(pyridin-3- | 495.1 | M + H |
| | ylcarbamoyl)amino]phenyl}amino)-1H-pyrazolo[3,4- | | |
| | d]pyrimidin-4-yl]amino}benzamide | | |
| 81 | 4-{[1-Methyl-6-({4-[({4-[(4-methylpiperazin-1- | 620.1 | M + H |
| | yl)carbonyl]phenyl}carbamoyl)amino]phenyl}amino)- | | |
| | 1H-pyrazolo[3,4-d]pyrimidin-4-yl]amino}benzamide | | |
| 82 | 4-({6-[(4-{[(4-{[4-(Dimethylamino)piperidin-1- | 648.6 | M + H |
| | yl]carbonyl}phenyl)carbamoyl]amino}phenyl)amino]-1- | | |
| | methyl-1H-pyrazolo[3,4-d]pyrimidin-4- | | |
| | yl}amino)benzamide | | |
| 83 | 4-({6-[(4-{[(4-{[3-(Dimethylamino)pyrrolidin-1- | 634.3 | M + H |
| | yl]carbonyl}phenyl)carbamoyl]amino}phenyl)amino]-1- | | |
| | methyl-1H-pyrazolo[3,4-d]pyrimidin-4- | | |
| | yl}amino)benzamide | | |
| 84 | 4-[(1-Methyl-6-{[4-({[4-(morpholin-4- | 607.5 | M + H |
| | ylcarbonyl)phenyl]carbamoyl}amino)phenyl]amino}-1H- | | |
| | pyrazolo[3,4-d]pyrimidin-4-yl)amino]benzamide | | |
| 85 | 4-({[4-({4-[(4-Carbamoylphenyl)amino]-1-methyl-1H- | 595.2 | M + H |
| | pyrazolo[3,4-d]pyrimidin-6- | | |
| | yl}amino)phenyl]carbamoyl}amino)-N-(2-hydroxyethyl)- | | |
| | N-methylbenzamide | | |
| 86 | 4-[(6-{[4-({[2- | 489.3 | M + H |
| | (Dimethylamino)ethyl]carbamoyl}amino)phenyl]amino}- | | |
| | 1-methyl-1H-pyrazolo[3,4-d]pyrimidin-4- | | |
| | yl)amino]benzamide | | |
| 87 | 4-({1-Methyl-6-[(4-{[(4-{[4-(propan-2-yl)piperazin-1- | 646.3 | (M − H) |
| | yl]carbonyl}phenyl)carbamoyl]amino}phenyl)amino]- | | |
| | 1H-pyrazolo[3,4-d]pyrimidin-4-yl}amino)benzamide | | |
| 88 | 4-{[6-({4- | 458.3 | M + H |
| | [(Cyclopropylcarbamoyl)amino]phenyl}amino)-1- | | |
| | methyl-1H-pyrazolo[3,4-d]pyrimidin-4- | | |
| | yl]amino}benzamide | | |
| 89 | 4-{[1-Methyl-6-({4-[(propan-2- | 460.3 | M + H |
| | ylcarbamoyl)amino]phenyl}amino)-1H-pyrazolo[3,4- | | |
| | d]pyrimidin-4-yl]amino}benzamide | | |
| 90 | N-Methyl-4-{[1-methyl-6-({4-[(pyridin-3- | 509.2 | M + H |
| | ylcarbamoyl)amino]phenyl}amino)-1H-pyrazolo[3,4- | | |
| | d]pyrimidin-4-yl]amino}benzamide | | |
| 91 | 4-({1-Methyl-6-[(4-{[(1-methylpiperidin-4- | 515.3 | M + H |
| | yl)carbamoyl]amino}phenyl)amino]-1H-pyrazolo[3,4- | | |
| | d]pyrimidin-4-yl}amino)benzamide | | |

### 4-({[4-({4-[(4-Carbamoylphenyl)amino]-1-methyl-1H-pyrazolo[3,4-d]pyrimidin-6-yl}amino)phenyl]carbamoyl}amino)-N-[2-(dimethylamino)ethyl]-N-methylbenzamide (Example 92): MS: m/z 622.3 (M+H)

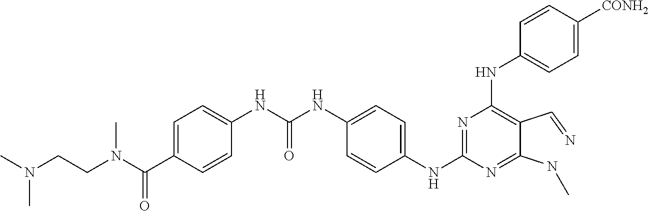

### N-(2-(Dimethylamino)ethyl)-N-methyl-4-nitrobenzamide

Into a solution of 4-nitrobenzoyl chloride (12 g, 64.7 mmol) in toluene (200 ml) was added dropwise N1,N1,N2-trimethylethane-1,2-diamine (10.09 mL, 78 mmol). The reaction mixture was vigorously stirred at room temperature for 14 hours, then suction filtered. The solid was partitioned between ethyl acetate and saturated NaHCO₃ aqueous solution. The organic layer was washed with saturated NaCl aqueous solution, dried over MgSO₄, suction filtered, concentrated and dried further in vacuo to give N-(2-(dimethylamino)ethyl)-N-methyl-4-nitrobenzamide (9.2 g, 36.6 mmol, 56.6%) as a white solid. MS (m/z): 252.2 (MH⁺).

### 4-Amino-N-(2-(dimethylamino)ethyl)-N-methylbenzamide

Into a solution of N-(2-(dimethylamino)ethyl)-N-methyl-4-nitrobenzamide (4 g, 15.92 mmol) in methanol (50 mL) was added Pd—C 10% (1 g, 0.940 mmol). The reaction flask was sealed with a rubber septa and a 2 L balloon of hydrogen gas was inserted. The reaction mixture was stirred under the hydrogen balloon pressure at room temperature for 14 hours. The resulting reaction mixture was suction filtered through a Celite™ bed. The filtrate was concentrated and dried further in vacuo to give 3.5 g of the desired product 4-amino-N-(2-(dimethylamino)ethyl)-N-methylbenzamide (3.5 g, 15.82 mmol, 99%) as a colorless gel. MS (m/z): 222.2 (MH+).

### N,N-Dimethyl-4-{[1-methyl-6-({4-[(pyridin-3-ylcarbamoyl)amino]phenyl}amino)-1H-pyrazolo[3,4-d]pyrimidin-4-yl]amino}benzamide (Example 93): MS: m/z 523.2 (M+H)

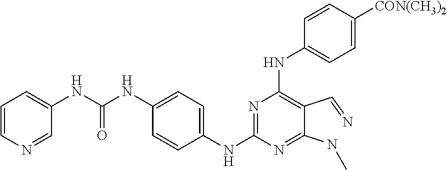

Preparation of 4-amino-N,N-dimethylbenzamide: To a solution of 4-nitrobenzoyl chloride (928 mg, 5 mmol) in THF (50 mL) was added dimethylamine (5.00 mL, 2M, 10.00 mmol) in THF. The mixture was stirred at room temperature for 2 hours and partitioned between EtOAc and saturated sodium carbonate solution. The organic phase was dried and filtered through Celite. Concentration led to the crude product, which was dissolved in methanol (50.0 mL). Pd—C (10%, 500 mg) was added, and the mixture was hydrogenated (with a balloon) for 3 hours. Filtration through Celite™ followed by concentration led to 522 mg of the desired product as a white solid.

### 1-[4-({1-Methyl-4-[(1-oxo-2,3-dihydro-1H-isoindol-5-yl)amino]-1H-pyrazolo[3,4-d]pyrimidin-6-yl}amino)phenyl]-3-pyridin-3-ylurea (Example 94): MS: m/z 507.2 (M+H)

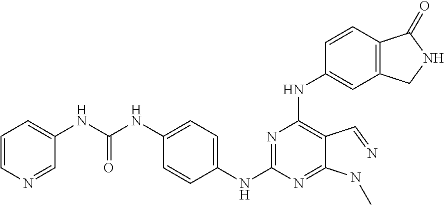

### Preparation of 5-amino-2,3-dihydro-1H-isoindol-1-one

A mixture of 2-methyl-4-nitrobenzoic acid (0.906 g, 5 mmol) in sulfurous dichloride (5.95 g, 50 mmol) was refluxed for 1 hour and concentrated. The residue was dissolved in methanol (30 mL, 741 mmol) and triethylamine (2 g, 19.76 mmol) was added. The mixture was stirred at room temperature for 1 hour, concentrated, and partitioned between ethyl acetate and water. The organic layer was separated, dried over MgSO4, and filtered through Magnesol™ to give 862 mg of methyl 2-methyl-4-benzoate as a tan solid.

A mixture of methyl 2-methyl-4-nitrobenzoate (696 mg, 3.57 mmol), 2,2′-azobis(2-methylpropionitrile (59 mg, 0.357 mmol), and N-bromosuccinimide (785 mg, 4.46 mmol) in CCl₄ (35 mL) in a sealed tube was flushed with N₂ for 5 minutes, and heated at 80° C. for 24 hours. The mixture was filtered through Celite™, and the filtrate was concentrated. To the residue was added 7 mL saturated ammonium in methanol. The mixture was stirred at room temperature for 2 hours, and concentrated. The residue was triturated with ethyl acetate, and the product was collected by filtration to give 100 mg of 5-nitro-2,3-dihydro-1H-isoindol-1-one as a light tan solid.

To a solution of 5-nitro-2,3-dihydro-1H-isoindol-1-one (60 mg, 0.337 mmol) in methanol (20 mL) was added 50 mg of 10% Pd on carbon. The mixture was hydrogenated (with a balloon) for 1 hour. Filtration through Celite™ followed by concentration led to 38 mg of 5-amino-2,3-dihydro-1H-isoindol-1-one as a tan solid.

### 1-[4-({1-Methyl-4-[(2-methyl-1-oxo-2,3-dihydro-1H-isoindol-5-yl)amino]-1H-pyrazolo[3,4-d]pyrimidin-6-yl}amino)phenyl]-3-pyridin-3-ylurea (Example 95): MS: m/z 521.2 (M+H)

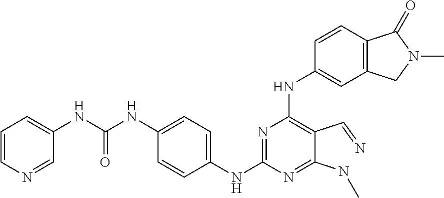

Preparation of 5-amino-2-methyl-2,3-dihydro-1H-isoindol-1-one was done analogously with the preparation of 5-amino-2,3-dihydro-1H-isoindol-1-one, using 33% methylamine in ethanol instead of saturated ammonium in methanol.

### Biological Evaluation—PI3K-Alpha and PI3K-Gamma Fluorescence Polarization Assay Protocols

The reaction buffer was 20 mM HEPES pH7.5, 2 mM MgCl₂, 0.05% CHAPS, and 0.01% βME (added fresh). The substrate solution was 40 μM PIP2 (diC8, Echelon, Salt Lake City Utah cat #P-4508, 1 mM in water) and 50 μM ATP in the reaction buffer. Nunc 384-well black polypropylene fluorescent plates were used for PI3K assays. The assay is run by putting 9.5 μl of freshly diluted enzyme in the reaction buffer per well, adding 0.5 μl of diluted drug or DMSO, and mixing. Then 10 μl of the substrate solution is added to each well to start the reaction. A final concentration of 20 μM PIP2 and 25 μM ATP in the reaction was used. Reactions were allowed to proceed for 30-60 minutes at room temperature. After 30-60 minutes, 20 μl of a solution of 10 nM TAMRA detector (Red detector probe-Echelon) and 2.5 uM of GST-murineGRP (1.5 mg/ml in 17% glycerol) was added per well to stop the reaction. The resulting solution was mixed well and allowed to stand for 90-110 minutes before reading plate. Assay Plates were read on Perkin-Elmer Envision plate readers with appropriate filters for Tamra [BODIPY-TMRI(1,3,4,5)P4]. Data obtained were used to calculate enzymatic activity and enzyme inhibition by inhibitor compounds. It is important to keep Red probe solutions dark. This procedure is adapted from Echelon Biosciences Inc procedure for their PI3-Kinase fluorescence polarization activity Assay kit Product number K-1100.

### mTOR Enzyme Assay

The routine human TOR assays with purified enzyme were performed in 96-well plates by DELFIA format as follows. Enzymes were first diluted in kinase assay buffer (10 mM HEPES (pH 7.4), 50 mM NaCl, 50 mM β-glycerophosphate, 10 mM MnCl₂, 0.5 mM DTT, 0.25 μM microcystin LR, and 100 μg/mL BSA). To each well, 12 μL of the diluted enzyme were mixed briefly with 0.5 μL test inhibitor or the control vehicle dimethylsulfoxide (DMSO). The kinase reaction was initiated by adding 12.5 μL kinase assay buffer containing ATP and His6-S6K to give a final reaction volume of 25 μL containing 800 ng/mL FLAG-TOR, 100 μM ATP and 1.25 μM His6-S6K. The reaction plate was incubated for 2 hours (linear at 1-6 hours) at room temperature with gentle shaking and then terminated by adding 25 μL Stop buffer (20 mM HEPES (pH 7.4), 20 mM EDTA, 20 mM EGTA). The DELFIA detection of the phosphorylated (Thr-389) His6-S6K was performed at room temperature using a monoclonal anti-P(T389)-p70S6K antibody (1A5, Cell Signaling) labeled with Europium-N1-ITC (Eu) (10.4 Eu per antibody, PerkinElmer). The DELFIA Assay buffer and Enhancement solution were purchased from PerkinElmer. 45 μL of the terminated kinase reaction mixture was transferred to a MaxiSorp plate (Nunc) containing 55 μL PBS. The His6-S6K was allowed to attach for 2 hours after which the wells were aspirated and washed once with PBS. 100 μL of DELFIA Assay buffer with 40 ng/mL Eu-P(T389)-S6K antibody was added. The antibody binding was continued for 1 hour with gentle agitation. The wells were then aspirated and washed 4 times with PBS containing 0.05% Tween-20 (PBST). 100 μL of DELFIA Enhancement solution was added to each well and the plates were read in a PerkinElmer Victor model plate reader. Data obtained were used to calculate enzymatic activity and enzyme inhibition by potential inhibitors

### In Vitro Cell Growth Assay

Cell lines used were human adenocarcinoma (LoVo), pancreatic (PC3), prostate (LNCap), breast (MDA468, MCF7), colon (HCT116), renal (HTB44 A498), and ovarian (OVCAR3) tumor cell lines. The tumor cells were plated in 96-well culture plates at approximately 3000 cells per well. One day following plating, various concentrations of inhibitors in DMSO were added to cells (final DMSO concentration in cell assays was 0.25%). Three days after drug treatment, viable cell densities were determined by cell mediated metabolic conversion of the dye MTS, a well-established indicator of cell proliferation in vitro. Cell growth assays were performed using kits purchased from Promega Corporation (Madison, Wis.), following the protocol provided by the vendor. Measuring absorbance at 490 nm generated MTS assay results. Compound effect on cell proliferation was assessed relative to untreated control cell growth. The drug concentration that conferred 50% inhibition of growth was determined as IC₅₀ (μM). IC₅₀ values of 20 nM to several μM were observed in the various tumor lines for compounds of this invention.

Table 3 shows the results of the described biological assays.

TABLE 3

| | PI3K-α | PI3K-β | PI3K-γ | PI3K-δ | |
|---|---|---|---|---|---|
| | avg | avg | avg | avg | mTOR avg |
| Example | IC₅₀ (nM) | IC₅₀ (nM) | IC₅₀ (nM) | IC₅₀ (nM) | IC₅₀ (nM) |
| 1 | 6108 | | 7000 | | |
| 2 | >10000 | | 5104 | | |
| 3 | 7281 | | 5022 | | |
| 4 | >10000 | | 10117 | | |
| 5 | 8308 | | 7000 | | |
| 6 | 3504 | | 7000 | | |
| 7 | >10000 | | 5918 | | |
| 8 | 6025 | | 7000 | | |
| 9 | >10000 | | 13062 | | |
| 10 | 6183 | | 4662 | | |
| 11 | 167 | | 1584 | | 950 |
| 13 | >10000 | | 7699 | | |
| 15 | >10000 | | 8297 | | |
| 17 | >10000 | | 5843 | | |
| 18 | >10000 | | 9716 | | |
| 19 | >10000 | | 5928 | | |
| 21 | 2814 | | 2460 | | |
| 23 | 2650 | | 2753 | | |
| 24 | 5446 | | 5249 | | |
| 25 | 3434 | | 2255 | | |
| 26 | 2248 | | 4000 | | |
| 30 | >5938 | | 3317 | | |
| 34 | 6700 | | >10000 | | 16000 |
| 35 | 1504 | | 7467 | | 4700 |
| 36 | >9750 | | | | |
| 38 | 298 | | 1144 | | 2500 |
| 39 | 160 | | 118 | | 2800 |
| 40 | 3778 | | >10000 | | |
| 41 | 215 | | 428 | | 7200 |
| 42 | 1210 | | 956 | | |
| 43 | 598 | | 1612 | | 8400 |
| 44 | 2872 | | >10000 | | |
| 45 | 653 | | 1292 | | >20,000 |
| 46 | 230 | 428 | 203 | 94 | >20,000 |
| 47 | 6044 | | >10000 | | |
| 49 | 258 | 1044 | 514 | 134 | >20,000 |
| 50 | 113 | 213 | 114 | 65 | 2100 |
| 51 | 4957 | | >10000 | | |
| 52 | 1332 | | 10500 | | |
| 53 | 4620 | | >10000 | | |
| 54 | 266 | | 398 | | 4900 |
| 55 | 387 | | 600 | | 570 |
| 56 | 11536 | | >10000 | | 16000 |
| 57 | 3216 | | >10000 | | 12000 |
| 58 | 3802 | | 10647 | | >20,000 |
| 59 | 2874 | | 2511 | | >20,000 |
| 60 | 8554 | | >10000 | | >20,000 |
| 61 | 2464 | | >10000 | | >20,000 |
| 62 | 603 | | 1587 | | 4800 |
| 63 | 75 | 2153 | 416 | 439 | 2800 |
| 64 | 40 | 1616 | 331 | 170 | 300 |
| 65 | 314 | | 1713 | | 1300 |
| 66 | 762 | | 1158 | | >20,000 |
| 67 | 660 | | 1256 | | 16000 |
| 68 | 1052 | | 1030 | | >20,000 |
| 69 | 10815 | | >10000 | | >20,000 |
| 72 | 580 | | 1348 | | >20,000 |
| 73 | 1761 | | >9600 | | 8200 |
| 74 | 637 | | 1344 | | 5500 |
| 75 | 1020 | | 1716 | | 9200 |
| 76 | 558 | | 570 | | 330 |
| 77 | 493 | | 2897 | | 1700 |
| 78 | 277 | | 3601 | | 775 |
| 79 | 1.0 | | 68 | | 13 |
| 80 | 529.0 | | 2009 | | 3450 |
| 81 | 0.4 | | 6 | | 17 |
| 82 | 0.2 | | 5 | | 7 |
| 83 | 0.3 | | 7 | | 5 |
| 84 | 0.7 | | 5 | | 7 |
| 85 | 0.4 | | 11 | | 9 |
| 86 | 10.0 | | 143 | | 2200 |
| 87 | 0.4 | | 5 | | 8 |
| 88 | 27.0 | | 263 | | 44 |
| 89 | 123.5 | | 523 | | 70 |
| 90 | 2.5 | | 75 | | 12 |
| 91 | 198.5 | | 3173 | | 2000 |
| 92 | <0.3 | | 6 | | 9 |
| 93 | 2.1 | | 75 | | 12 |
| 94 | 1.6 | | 11 | | 20 |
| 95 | 6.0 | | 148 | | 68 |

Throughout this application, various publications are referenced. The disclosures of these publications in their entireties are hereby incorporated by reference into this application in order to more fully describe the state of the art as known to those skilled therein as of the date of the invention described and claimed herein.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1) A compound of Formula I: 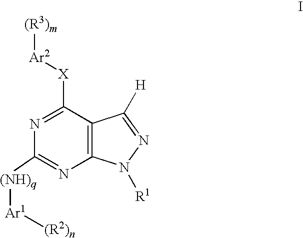
or a pharmaceutically acceptable salt thereof wherein;
q is 0 or 1 with the proviso that when q=1, then n is 1, 2, or 3 and at least one of R² is R⁶R⁷NC(O)NH—,
Ar¹ and Ar² are each independently phenyl, naphthyl, 1-oxo-2,3-dihydro-1H-isoindol-5-yl, or a nitrogen-containing mono- or bicyclic heteroaryl-;
X is —NH—, —N(C₁-C₆alkyl)-, —O—, or —S—;
R¹ is selected from: a) hydrogen; b) C₁-C₆alkyl- optionally substituted with from 1 to 3 substituents independently selected from: i) C₁-C₆alkoxy-, ii) (C₁-C₆alkyl)amino-, iii) di(C₁-C₆alkyl)amino-, iv) HC(O)—, v) HO₂C—, and vi) (C₁-C₆alkoxy)carbonyl-; c) C₁-C₆aminoalkyl- optionally substituted with from 1 to 3 substituents independently selected from: i) C₆-C₁₄aryl- optionally substituted with halogen, ii) (C₁-C₉heteroaryl)alkyl-, iii) (C₆-C₁₄aryl)alkyl-, iv) H₂N—C₁-C₆alkylene-, v) (C₁-C₆alkyl)amino-C₁-C₆alkylene-, and vi) di(C₁-C₆alkyl)amino-C₁-C₆alkylene-; d) carbonylamidoalkyl- optionally substituted with a substituent selected from: i) halogen, or ii) di(C₁-C₆alkyl)amino-; e) C₃-C₈cycloalkyl- optionally substituted with from 1 to 3 substituents independently selected from: i) C₁-C₆alkoxy-, ii) (C₁-C₆alkyl)amino-, iii) di(C₁-C₆alkyl)amino-, iv) HC(O)—, v) HO₂C—, and vi) (C₁-C₆alkoxy)carbonyl-; f) C₆-C₁₄aryl- optionally substituted with a substituent selected from: i) HO₂C—, ii) C₁-C₆hydroxylalkyl-, iii) R⁴R⁵NC(O)—, or iv) (C₁-C₆alkoxy)carbonyl-; g) C₁-C₉heterocycle- optionally substituted with from 1 to 3 substituents independently selected from: i) C₁-C₈acyl-, wherein the C₁-C₈acyl- is optionally substituted with a H₂N—, ii) C₁-C₆alkyl-, iii) (C₁-C₉heteroaryl)alkyl- wherein the ring portion of the (C₁-C₉heteroaryl)alkyl- group is optionally substituted with from 1 to 3 substituents independently selected from: (A) C₁-C₆alkylC(O)NH—, (B) halogen, (C) H₂N—, and (D) C₁-C₆alkyl-, iv) C₁-C₉heterocyclyl(C₁-C₆alkyl)-, wherein the ring portion of the C₁-C₉heterocyclyl(C₁-C₆alkyl)- group is optionally substituted by a (C₆-C₁₄aryl)alkyl-, v) (C₆-C₁₄aryl)alkyl-, wherein the ring portion of the (C₆-C₁₄aryl)alkyl- group is optionally substituted by 1 to 3 substituents independently selected from: (A) halogen, (B) C₁-C₆alkyl-, (C) di(C₁-C₆alkyl)amino-(C₁-C₆alkylene)-O—, and (D) and C₁-C₉heteroaryl-; and vi) (C₁-C₆alkoxy)carbonyl-; h) C₁-C₉heterocyclyl(C₁-C₆alkyl)- optionally substituted with a substituent selected from: i) C₁-C₆alkyl-, ii) C₃-C₈cycloalkyl-, iii) (C₁-C₆alkoxy)carbonyl-, iv) C₁-C₆alkylcarboxy-, v) (C₆-C₁₄aryl)alkyl- wherein the ring portion of the (C₆-C₁₄aryl)alkyl- group is optionally substituted with a substituent selected from: (A) halogen, (B) C₁-C₉heteroaryl-, or (C) di(C₁-C₆alkyl)amino-(C₁-C₆alkylene)-O—, vi) (C₁-C₉heteroaryl)alkyl- wherein the ring portion of the (C₁-C₉heteroaryl)alkyl- group is optionally substituted by a halogen, or vii) C₁-C₈acyl-, wherein the C₁-C₈acyl- is optionally substituted with from 1 to 3 independently selected halogens, i) (C₁-C₉heteroaryl)alkyl- wherein the ring portion of the (C₁-C₉heteroaryl)alkyl- is optionally substituted by 1 to 3 substituents independently selected from: i) R⁴R⁵NC(O)NH—, ii) (C₁-C₆alkoxy)carbonyl-, iii) HO₂C—, iv) hydroxyl, and v) R⁴R⁵NC(O)—; j) (C₆-C₁₄aryl)alkyl- wherein the ring portion of the (C₆-C₁₄aryl)alkyl- group is optionally by 1 to 3 substituents independently selected from: i) R⁴R⁵NC(O)NH—, ii) (C₁-C₆alkoxy)carbonyl-, iii) HO₂C—, iv) hydroxyl, v) and R⁴R⁵NC(O)—; k) C₁-C₆hydroxylalkyl-; l) C₁-C₆perfluoroalkyl-; or m) C₁-C₉heteroaryl- optionally substituted with a substituent selected from: i) HO₂C—, ii) C₁-C₆hydroxylalkyl-, iii) R⁴R⁵NC(O)—, or iv) (C₁-C₆alkoxy)carbonyl-;
R⁴ and R⁵ are each independently selected from: a) H; b) C₁-C₆alkyl- optionally substituted with a substituent selected from: i) C₁-C₆alkylC(O)NH—, ii) H₂N—, iii) (C₁-C₆alkyl)amino-, or iv) di(C₁-C₆alkyl)amino-; c) C₃-C₈cycloalkyl- optionally substituted with a substituent selected from: i) C₁-C₆alkylC(O)NH—, ii) H₂N—, iii) (C₁-C₆alkyl)amino-, or iv) or di(C₁-C₆alkyl)amino-; d) C₆-C₁₄aryl- optionally substituted with a substituent selected from: i) halogen, or ii) monocyclic C₁-C₆heterocycle- wherein the monocyclic C₁-C₆heterocycle- is optionally substituted with (C₁-C₆alkoxy)carbonyl-; e) C₁-C₉heteroaryl-; f) (C₁-C₉heteroaryl)alkyl-; g) C₁-C₉heterocyclyl(C₁-C₆alkyl)-; h) (C₆-C₁₄aryl)alkyl-, wherein the chain portion of the (C₆-C₁₄aryl)alkyl- group is optionally substituted by a hydroxyl; or i) monocyclic C₁-C₆heterocycle- optionally substituted with a (C₁-C₆alkoxy)carbonyl-;
or R⁴ and R⁵, when taken together with the nitrogen to which they are attached, form a 3- to 7-membered heterocycle- wherein up to two of the carbon atoms of the heterocycle- are optionally replaced with —N(H)—, —N(C₁-C₆alkyl)-, —N(C₆-C₁₄aryl)-, —S—, —SO—, —S(O)₂, or —O—;
R² and R³ are each independently selected from: a) C₁-C₈acyl-, b) C₁-C₆alkyl-, which is optionally substituted with from 1 to 3 substituents independently selected from: i) H₂N—, ii) (C₁-C₆alkyl)amino-, iii) di(C₁-C₆alkyl)amino-, and iv) C₁-C₉heterocyclyl-, c) (C₁-C₆alkyl)amido-, d) (C₁-C₆alkyl)carboxyl-, e) (C₁-C₆alkyl)carbonylamido-, f) C₁-C₆alkoxy- optionally substituted by C₁-C₆alkoxy- or C₁-C₉heteroaryl-, g) (C₁-C₆alkoxy)carbonyl-, h) (C₆-C₁₄aryl)alkyl-O—, wherein the ring portion of the (C₆-C₁₄aryl)alkyl-O— group is optionally substituted with from 1 to 3 substituents independently selected from: i) C₁-C₆alkoxy-, and ii) halogen, i) C₃-C₈cycloalkyl-, j) halogen, k) C₁-C₆haloalkyl-, l) C₁-C₉heterocyclyl- optionally substituted by C₁-C₆alkyl- or C₁-C₆hydroxylalkyl-, m) C₁-C₉heterocyclyl(C₁-C₆alkyl)- optionally substituted by C₁-C₆alkyl-, n) hydroxyl, o) C₁-C₆hydroxylalkyl-, p) C₁-C₆perfluoroalkyl-, q) C₁-C₆perfluoroalkyl-O—, r) R⁶R⁷N—, s) C₁-C₉heterocyclyl-, t) CN, u) HO₂C—, v) R⁶R⁷NC(O)—, w) C₁-C₉heterocyclyl-C(O)—, x) R⁶C(O)NH—, y) R⁶R⁷NS(O)₂—, z) R⁶R⁷NC(O)NHC(O)NH—, aa) R⁸OC(O)NHC(O)NH—, bb) C₁-C₆alkoxy-C₁-C₆alkylene-NH—C₁-C₆alkylene-, cc) C₁-C₆hydroxylalkyl-NH—C₁-C₆alkylene-, dd) amino(C₁-C₆alkyl)-NH—C₁-C₆alkylene-, ee) di(C₁-C₆alkyl)amino-C₁-C₆alkylene-NH—C₁-C₆alkylene-, C₁— ff) C₆hydroxylalkyl-NH—, gg) amino(C₁-C₆alkyl)-NH—, hh) (C₁-C₆alkyl) N-alkylamido-, ii) R⁶R⁷NC(O)NH—, jj) C₁-C₉heterocyclyl-C(O)NH—, kk) R⁸OC(O)NH—, ll) R⁸S(O)₂NH—, mm) R⁸S(O)₂—, nn) —C(═N—(OR⁶))—(NR⁶R⁷), or oo) O₂N—;
R⁶ and R⁷ are each independently selected from: H; C₁-C₆alkyl- optionally substituted with from 1 to 3 substituents independently selected from C₁-C₆alkoxy-, H₂N—, (C₁-C₆alkyl)amino-, di(C₁-C₆alkyl)amino-, C₆-C₁₄aryl-, C₁-C₉heterocyclyl- optionally substituted by C₁-C₆alkyl-, and C₁-C₉heteroaryl-; C₁-C₆alkoxy; C₁-C₉heteroaryl- optionally substituted with from 1 to 3 substituents independently selected from C₁-C₆alkyl- optionally substituted with H₂N—, (C₁-C₆alkyl)amino-, or di(C₁-C₆alkyl)amino-, C₁-C₉heterocyclyl(C₁-C₆alkyl)-, halogen, hydroxyl, H₂N—, O₂N—, H₂NSO₂—, HO₂C—, (C₁-C₆alkoxy)carbonyl-, (C₁-C₆alkoxy)C(O)NH—, (C₁-C₆alkyl)amino-, di(C₁-C₆alkyl)amino-, R⁹R¹⁰NC(O)—, R⁹O—, R⁹R¹⁰N—, R⁹R¹⁰NS(O)₂—, R⁹S(O)₂NR¹⁰—, R⁹R¹⁰NC(O)NH—, R⁹S—, R⁹S(O)—, R⁹S(O)₂—, R⁹C(O)—, C₁-C₉heterocyclyl- optionally substituted by C₁-C₆alkyl- or C₁-C₆hydroxylalkyl-, C₁-C₆hydroxylalkyl-, and perfluoro(C₁-C₆)alkyl-; C₁-C₆hydroxylalkyl-; C₁-C₉heterocyclyl-; C₆-C₁₄aryl- optionally substituted with from 1 to 3 substituents independently selected from C₁-C₆alkyl- optionally substituted with H₂N—, (C₁-C₆alkyl)amino-, or di(C₁-C₆alkyl)amino-, C₁-C₉heterocyclyl(C₁-C₆alkyl)-, halogen, hydroxyl, H₂N—, O₂N—, H₂NSO₂—, HO₂C—, (C₁-C₆alkoxy)carbonyl-, (C₁-C₆alkoxy)C(O)NH—, (C₁-C₆alkyl)amino-, di(C₁-C₆alkyl)amino-, R⁹R¹⁰NC(O)—, Z, wherein Z is R⁹O—, R⁹R¹⁰N—, R⁹R¹⁰NS(O)₂—, R⁹S(O)₂NR¹⁷—, R⁹R¹⁰NC(O)NH—, R⁹S—, R⁹S(O)—, R⁹S(O)₂—, R⁹C(O)—, C₁-C₉heterocyclyl- optionally substituted by C₁-C₆alkyl- or C₁-C₆hydroxylalkyl-, C₁-C₆hydroxylalkyl-, or perfluoro(C₁-C₆)alkyl-; and C₃-C₈cycloalkyl-;
or R⁶ and R⁷, when taken together with the nitrogen to which they are attached, form a 3- to 7-membered heterocycle- wherein up to two of the carbon atoms of the heterocycle- are optionally replaced with —N(H)—, —N(C₁-C₆alkyl)-, —N(C₆-C₁₄aryl)-, —S—, —SO—, —S(O)_{2—}, or —O—;
R⁸ is selected from C₁-C₆alkyl-; C₆-C₁₄aryl-; (C₆-C₁₄aryl)alkyl-, optionally substituted by H₂N—; C₁-C₉heterocyclyl-; C₃-C₈cycloalkyl-; C₁-C₆hydroxylalkyl-; and C₁-C₆perfluoroalkyl-;
R⁹ and R¹⁰ are each independently selected from: H; C₁-C₆alkyl-; (C₁-C₆alkyl)amino-C₂-C₆alkylene-; di(C₁-C₆alkyl)amino-C₂-C₆alkylene-; C₂-C₆alkenyl; C₂-C₆alkynyl; C₆-C₁₄aryl-; (C₆-C₁₄aryl)alkyl-; C₃-C₈cycloalkyl-; C₁-C₆hydroxylalkyl-; C₁-C₉heteroaryl-; (C₁-C₉heteroaryl)alkyl; C₁-C₉heterocyclyl-; and C₁-C₉heterocyclyl(C₁-C₆alkyl)-;
or R⁹ and R¹⁰, when taken together with the nitrogen to which they are attached, form a 3- to 7-membered heterocycle- wherein up to two of the carbon atoms of the heterocycle- are optionally replaced with —N(H)—, —N(C₁-C₆alkyl)-, —N(C₆-C₁₄aryl)-, —S—, —SO—, —S(O)₂, or —O— and the heterocycle is optionally substituted by H₂N—, (C₁-C₆alkyl)amino-, or di(C₁-C₆alkyl)amino-;
m and n are each independently 0, 1, 2, or 3;
except that 2-[(1-methyl-6-phenyl-1H-pyrazolo[3,4-d]pyrimidin-4-yl)amino]-benzoic acid, 2-[(1-ethyl-6-phenyl-1H-pyrazolo[3,4-d]pyrimidin-4-yl)amino]-benzoic acid, 2-[(1-propyl-6-phenyl-1H-pyrazolo[3,4-d]pyrimidin-4-yl)amino]-benzoic acid, 2-[(1-methyl-6-phenyl-1H-pyrazolo[3,4-d]pyrimidin-4-yl)amino]-benzoic acid methyl ester, 2-[(1-methyl-6-phenyl-1H-pyrazolo[3,4-d]pyrimidin-4-yl)amino]-benzoic acid ethyl ester, 1-methyl-6-phenyl-4-(phenylthio)-1H-pyrazolo[3,4-d]pyrimidine, 1-ethyl-6-phenyl-4-(phenylthio)-1H-pyrazolo[3,4-d]pyrimidine, and 1-propyl-6-phenyl-4-(phenylthio)-1H-pyrazolo[3,4-d]pyrimidine are excluded;
and when R¹ is phenyl, X is —NH—, and m is 2, then (R²)ₙ is not monofluoro.

2) Compounds of claim 1 of the Formula II: 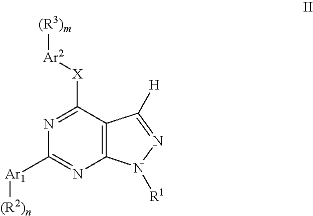
or a pharmaceutically acceptable salt thereof wherein; the constituent variables are as defined above for claim 1.

3) Compounds of claim 1 of the Formula III: 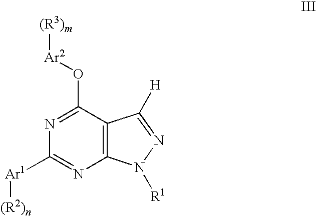
or pharmaceutically acceptable salts thereof, wherein the constituent variables are as defined above in claim 1.

4) Compounds of claim 1 of the Formula IV: 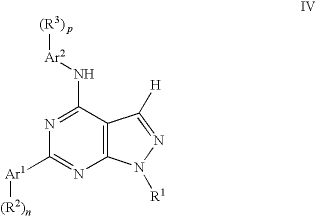
or pharmaceutically acceptable salts thereof, wherein:
Ar² is phenyl or indolyl;
R³ is independently selected from: a) C₁-C₈acyl-, b) C₁-C₆alkyl-, which is substituted with from 1 to 3 substituents independently selected from: i) H₂N—, ii) (C₁-C₆alkyl)amino-, iii) di(C₁-C₆alkyl)amino-, and iv) C₁-C₉heterocyclyl-, c) (C₁-C₆alkyl)amido-, d) (C₁-C₆alkyl)carboxyl-, e) (C₁-C₆alkyl)carbonylamido-, f) C₁-C₆alkoxy- optionally substituted by C₁-C₆alkoxy- or C₁-C₉heteroaryl-, g) (C₁-C₆alkoxy)carbonyl-, h) (C₆-C₁₄aryl)alkyl-O—, wherein the ring portion of the (C₆-C₁₄aryl)alkyl-O— group is optionally substituted with from 1 to 3 substituents independently selected from: i) C₁-C₆alkoxy-, and ii) halogen, i) C₃-C₈cycloalkyl-, j) C₁-C₆haloalkyl-, k) C₁-C₉heterocyclyl- optionally substituted by C₁-C₆alkyl- or C₁-C₆hydroxylalkyl-, l) C₁-C₉heterocyclyl(C₁-C₆alkyl)- optionally substituted by C₁-C₆alkyl-, m) hydroxyl, n) C₁-C₆hydroxylalkyl-, o) R⁶R⁷N—, p) C₁-C₉heterocyclyl-, q) CN, r) HO₂C—, s) H₂NC(O)— t) C₁-C₉heterocyclyl-C(O)—, u) R⁶C(O)NH—, v) R⁶R⁷NS(O)₂—, w) R⁶R⁷NC(O)NHC(O)NH—, x) R⁸OC(O)NHC(O)NH—, y) C₁-C₆alkoxy-C₁-C₆alkylene-NH—C₁-C₆alkylene-, z) C₁-C₆hydroxylalkyl-NH—C₁-C₆alkylene-, aa) amino(C₁-C₆alkyl)-NH—C₁-C₆alkylene-, bb) di(C₁-C₆alkyl)amino-C₁-C₆alkylene-NH—C₁-C₆alkylene-, cc) C₁-C₆hydroxylalkyl-NH—, dd) amino(C₁-C₆alkyl)-NH—, ee) (C₁-C₆alkyl) N-alkylamido-, ff) R⁶R⁷NC(O)NH—, gg) C₁-C₉heterocyclyl-C(O)NH—, hh) R⁸OC(O)NH—, ii) R⁸S(O)₂NH—, jj) R⁸S(O)₂—, kk) —C(═N—(OR⁶))—(NR⁶R⁷), or ll) O₂N—;
p is 1, 2, or 3;
and the remaining constituent variables are as defined above in claim 1

5) Compounds of claim 1 of the Formula V: 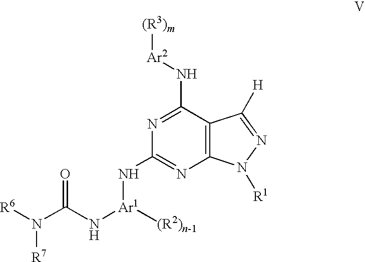
or a pharmaceutically acceptable salt thereof, wherein (n-1) is 0, 1, or 2 and the remaining constituent variables are as defined above in claim 1.

6) Compounds of claim 1 wherein, X is —NH—.

7) Compounds of claim 5 wherein, Ar¹ and Ar² are phenyl.

8) Compounds of claim 7 wherein, R¹ is C₁-C₆alkyl.

9) Compounds of claim 8 wherein, R¹ is CH₃.

10) Compounds of claim 9 wherein, (n-1) is 0.

11) Compounds of claim 10 wherein, m is 1.

12) Compounds of claim 11 wherein, R³ is H₂NC(O)—.

13) Compounds of claim 12 wherein, R⁶ is C₆-C₁₄aryl- substituted with R⁹R¹⁰NC(O)—.

14) A compound selected from the group consisting of:
N-(3,4-dimethoxyphenyl)-N,1-dimethyl-6-phenyl-1H-pyrazolo[3,4-d]pyrimidin-4-amine;
N-(4-methoxyphenyl)-N,1-dimethyl-6-phenyl-1H-pyrazolo[3,4-d]pyrimidin-4-amine;
4-(1H-indol-5-yloxy)-6-(3-methoxyphenyl)-1-methyl-1H-pyrazolo[3,4-d]pyrimidine;
6-(3,4-dimethoxyphenyl)-4-(1H-indol-5-yloxy)-1-methyl-1H-pyrazolo[3,4-d]pyrimidine;
4-[4-(1H-indol-5-yloxy)-1-methyl-1H-pyrazolo[3,4-d]pyrimidin-6-yl]-N,N-dimethylaniline;
4-(1H-indol-5-yloxy)-1-methyl-6-pyridin-3-yl-1H-pyrazolo[3,4-d]pyrimidine;
4-(1H-indol-5-yloxy)-1-methyl-6-[3-(trifluoromethyl)phenyl]-1H-pyrazolo[3,4-d]pyrimidine;
3-[4-(1H-indol-5-yloxy)-1-methyl-1H-pyrazolo[3,4-d]pyrimidin-6-yl]benzonitrile;
6-biphenyl-3-yl-4-(1H-indol-5-yloxy)-1-methyl-1H-pyrazolo[3,4-d]pyrimidine;
4-(3,4-dimethoxyphenoxy)-6-(3-methoxyphenyl)-1-methyl-1H-pyrazolo[3,4-d]pyrimidine;
4-(3,4-dimethoxyphenoxy)-6-(3,4-dimethoxyphenyl)-1-methyl-1H-pyrazolo[3,4-d]pyrimidine;
4-[4-(3,4-dimethoxyphenoxy)-1-methyl-1H-pyrazolo[3,4-d]pyrimidin-6-yl]-N,N-dimethylaniline;
6-biphenyl-3-yl-4-(3,4-dimethoxyphenoxy)-1-methyl-1H-pyrazolo[3,4-d]pyrimidine;
N-(4-{[6-(3-methoxyphenyl)-1-methyl-1H-pyrazolo[3,4-d]pyrimidin-4-yl]oxy}phenyl)acetamide;
N-(4-{[6-(3-hydroxyphenyl)-1-methyl-1H-pyrazolo[3,4-d]pyrimidin-4-yl]oxy}phenyl)acetamide;
N-[4-({6-[4-(dimethylamino)phenyl]-1-methyl-1H-pyrazolo[3,4-d]pyrimidin-4-yl}oxy)phenyl]acetamide;
N-[4-({1-methyl-6-[3-(trifluoromethyl)phenyl]-1H-pyrazolo[3,4-d]pyrimidin-4-yl}oxy)phenyl]acetamide;
N-{4-[(6-biphenyl-3-yl-1-methyl-1H-pyrazolo[3,4-d]pyrimidin-4-yl)oxy]phenyl}acetamide;
N-(4-{[6-(4-hydroxyphenyl)-1-methyl-1H-pyrazolo[3,4-d]pyrimidin-4-yl]oxy}phenyl)acetamide;
N-(4-{[6-(1H-indol-5-yl)-1-methyl-1H-pyrazolo[3,4-d]pyrimidin-4-yl]oxy}phenyl)acetamide;
6-biphenyl-4-yl-4-(1H-indol-5-yloxy)-1-methyl-1H-pyrazolo[3,4-d]pyrimidine;
4-(1H-indol-5-yloxy)-6-(6-methoxy-2-naphthyl)-1-methyl-1H-pyrazolo[3,4-d]pyrimidine;
4-(1H-indol-5-yloxy)-6-(4-isopropylphenyl)-1-methyl-1H-pyrazolo[3,4-d]pyrimidine;
4-(1H-indol-5-yloxy)-1-methyl-6-(1-methyl-1H-pyrazol-4-yl)-1H-pyrazolo[3,4-d]pyrimidine;
6-(3-chlorophenyl)-4-(1H-indol-5-yloxy)-1-methyl-1H-pyrazolo[3,4-d]pyrimidine;
4-(1H-indol-5-yloxy)-1-methyl-6-(3-nitrophenyl)-1H-pyrazolo[3,4-d]pyrimidine;
6-(3-furyl)-4-(1H-indol-5-yloxy)-1-methyl-1H-pyrazolo[3,4-d]pyrimidine;
6-{3-[(2-chlorobenzyl)oxy]phenyl}-4-(1H-indol-5-yloxy)-1-methyl-1H-pyrazolo[3,4-d]pyrimidine;
6-{4-[(3,5-dimethoxybenzyl)oxy]phenyl}-4-(1H-indol-5-yloxy)-1-methyl-1H-pyrazolo[3,4-d]pyrimidine;
6-{3-[(3,5-dimethoxybenzyl)oxy]phenyl}-4-(1H-indol-5-yloxy)-1-methyl-1H-pyrazolo[3,4-d]pyrimidine;
6-(3,5-dimethylisoxazol-4-yl)-4-(1H-indol-5-yloxy)-1-methyl-1H-pyrazolo[3,4-d]pyrimidine;
4-(3,4-dimethoxyphenoxy)-1-methyl-6-phenyl-1H-pyrazolo[3,4-d]pyrimidine;
N-{4-[(1-methyl-6-phenyl-1H-pyrazolo[3,4-d]pyrimidin-4-yl)oxy]phenyl}acetamide;
4-[(1-methyl-6-phenyl-1H-pyrazolo[3,4-d]pyrimidin-4-yl)oxy]benzamide; and
4-(1H-indol-5-yloxy)-1-methyl-6-phenyl-1H-pyrazolo[3,4-d]pyrimidine; and pharmaceutically acceptable salts thereof.

15) A compound selected from the group consisting of:
4-{[1-(1-benzylpiperidin-4-yl)-6-phenyl-1H-pyrazolo[3,4-d]pyrimidin-4-yl]amino}benzamide;
1-(1-benzylpiperidin-4-yl)-N-(3,4-dimethoxyphenyl)-6-phenyl-1H-pyrazolo[3,4-d]pyrimidin-4-amine;
3-{[1-(1-benzylpiperidin-4-yl)-6-phenyl-1H-pyrazolo[3,4-d]pyrimidin-4-yl]amino}benzamide;
1-(1-benzylpiperidin-4-yl)-N-1H-indol-5-yl-6-phenyl-1H-pyrazolo[3,4-d]pyrimidin-4-amine;
N-(3-{[1-(1-benzylpiperidin-4-yl)-6-phenyl-1H-pyrazolo[3,4-d]pyrimidin-4-yl]amino}phenyl)acetamide;
4-{[1-(1-benzylpiperidin-4-yl)-6-(3-hydroxyphenyl)-1H-pyrazolo[3,4-d]pyrimidin-4-yl]amino}benzamide;
3-{1-(1-benzylpiperidin-4-yl)-4-[(3,4-dimethoxyphenyl)amino]-1H-pyrazolo[3,4-d]pyrimidin-6-yl}phenol;
3-{[1-(1-benzylpiperidin-4-yl)-6-(3-hydroxyphenyl)-1H-pyrazolo[3,4-d]pyrimidin-4-yl]amino}benzamide;
3-[1-(1-benzylpiperidin-4-yl)-4-(1H-indol-5-ylamino)-1H-pyrazolo[3,4-d]pyrimidin-6-yl]phenol;
N-(3-{[1-(1-benzylpiperidin-4-yl)-6-(3-hydroxyphenyl)-1H-pyrazolo[3,4-d]pyrimidin-4-yl]amino}phenyl)acetamide;
4-[(1-methyl-6-phenyl-1H-pyrazolo[3,4-d]pyrimidin-4-yl)amino]benzamide;
4-[(6-phenyl-1-piperidin-4-yl-1H-pyrazolo[3,4-d]pyrimidin-4-yl)amino]benzamide;
N-(3,4-dimethoxyphenyl)-6-phenyl-1-piperidin-4-yl-1H-pyrazolo[3,4-d]pyrimidin-4-amine;
3-[(6-phenyl-1-piperidin-4-yl-1H-pyrazolo[3,4-d]pyrimidin-4-yl)amino]benzamide;
N-{3-[(6-phenyl-1-piperidin-4-yl-1H-pyrazolo[3,4-d]pyrimidin-4-yl)amino]phenyl}acetamide;
4-{[6-(3-hydroxyphenyl)-1-piperidin-4-yl-1H-pyrazolo[3,4-d]pyrimidin-4-yl]amino}benzamide;
3-{4-[(3,4-dimethoxyphenyl)amino]-1-piperidin-4-yl-1H-pyrazolo[3,4-d]pyrimidin-6-yl}phenol;
3-{[6-(3-hydroxyphenyl)-1-piperidin-4-yl-1H-pyrazolo[3,4-d]pyrimidin-4-yl]amino}benzamide;
3-[4-(1H-indol-5-ylamino)-1-piperidin-4-yl-1H-pyrazolo[3,4-d]pyrimidin-6-yl]phenol;
N-(3-{[6-(3-hydroxyphenyl)-1-piperidin-4-yl-1H-pyrazolo[3,4-d]pyrimidin-4-yl]amino}phenyl)acetamide;
4-{[1-(1-benzylpiperidin-4-yl)-6-(3-nitrophenyl)-1H-pyrazolo[3,4-d]pyrimidin-4-yl]amino}benzamide;
4-({1-(1-benzylpiperidin-4-yl)-6-[4-(hydroxymethyl)phenyl]-1H-pyrazolo[3,4-d]pyrimidin-4-yl}amino)benzamide;
4-{[1-(1-benzylpiperidin-4-yl)-6-(3-methoxyphenyl)-1H-pyrazolo[3,4-d]pyrimidin-4-yl]amino}benzamide;
4-{[1-(1-benzylpiperidin-4-yl)-6-(4-hydroxyphenyl)-1H-pyrazolo[3,4-d]pyrimidin-4-yl]amino}benzamide;
4-({1-(1-benzylpiperidin-4-yl)-6-[3-(trifluoromethyl)phenyl]-1H-pyrazolo[3,4-d]pyrimidin-4-yl}amino)benzamide;
4-{[1-(1-benzylpiperidin-4-yl)-6-(2-hydroxyphenyl)-1H-pyrazolo[3,4-d]pyrimidin-4-yl]amino}benzamide;
1-(1-benzylpiperidin-4-yl)-N-(3,4-dimethoxyphenyl)-6-(3-nitrophenyl)-1H-pyrazolo[3,4-d]pyrimidin-4-amine;
(4-{1-(1-benzylpiperidin-4-yl)-4-[(3,4-dimethoxyphenyl)amino]-1H-pyrazolo[3,4-d]pyrimidin-6-yl}phenyl)methanol;
1-(1-benzylpiperidin-4-yl)-N-(3,4-dimethoxyphenyl)-6-(3-methoxyphenyl)-1H-pyrazolo[3,4-d]pyrimidin-4-amine;
4-{1-(1-benzylpiperidin-4-yl)-4-[(3,4-dimethoxyphenyl)amino]-1H-pyrazolo[3,4-d]pyrimidin-6-yl}phenol;
1-(1-benzylpiperidin-4-yl)-N-(3,4-dimethoxyphenyl)-6-[3-(trifluoromethyl)phenyl]-1H-pyrazolo[3,4-d]pyrimidin-4-amine;
2-{1-(1-benzylpiperidin-4-yl)-4-[(3,4-dimethoxyphenyl)amino]-1H-pyrazolo[3,4-d]pyrimidin-6-yl}phenol;
N,1-dimethyl-N,6-diphenyl-1H-pyrazolo[3,4-d]pyrimidin-4-amine;
4-[methyl(1-methyl-6-phenyl-1H-pyrazolo[3,4-d]pyrimidin-4-yl)amino]benzamide;
N-{4-[methyl(1-methyl-6-phenyl-1H-pyrazolo[3,4-d]pyrimidin-4-yl)amino]phenyl}acetamide;
4-[(6-biphenyl-4-yl-1-methyl-1H-pyrazolo[3,4-d]pyrimidin-4-yl)amino]benzamide;
4-{[6-(4-isopropylphenyl)-1-methyl-1H-pyrazolo[3,4-d]pyrimidin-4-yl]amino}benzamide;
4-{[1-methyl-6-(1-methyl-1H-pyrazol-4-yl)-1H-pyrazolo[3,4-d]pyrimidin-4-yl]amino}benzamide;
4-{[6-(3,5-dimethylisoxazol-4-yl)-1-methyl-1H-pyrazolo[3,4-d]pyrimidin-4-yl]amino}benzamide;
4-{[6-(3-chlorophenyl)-1-methyl-1H-pyrazolo[3,4-d]pyrimidin-4-yl]amino}benzamide;
4-{[6-(3-furyl)-1-methyl-1H-pyrazolo[3,4-d]pyrimidin-4-yl]amino}benzamide;
4-(1-methyl-6-(4-(3-methylureido)phenyl)-1H-pyrazolo[3,4-d]pyrimidin-4-ylamino)benzamide;
4-(1-methyl-6-(3-(3-methylureido)phenyl)-1H-pyrazolo[3,4-d]pyrimidin-4-ylamino)benzamide; and
pharmaceutically acceptable salts thereof.

16) A compound selected from the group consisting of:
4-{[1-Methyl-6-({4-[(pyridin-3-ylcarbamoyl)amino]phenyl}amino)-1H-pyrazolo[3,4-d]pyrimidin-4-yl]amino}benzamide;
4-{[1-Methyl-6-({3-[(pyridin-3-ylcarbamoyl)amino]phenyl}amino)-1H-pyrazolo[3,4-d]pyrimidin-4-yl]amino}benzamide;
4-{[1-Methyl-6-({4-[({4-[(4-methylpiperazin-1-yl)carbonyl]phenyl}carbamoyl)amino]phenyl}amino)-1H-pyrazolo[3,4-d]pyrimidin-4-yl]amino}benzamide;
4-({6-[(4-{[(4-{[4-(Dimethylamino)piperidin-1-yl]carbonyl}phenyl)carbamoyl]amino}phenyl)amino]-1-methyl-1H-pyrazolo[3,4-d]pyrimidin-4-yl}amino)benzamide;
4-({6-[(4-{[(4-{[3-(Dimethylamino)pyrrolidin-1-yl]carbonyl}phenyl)carbamoyl]amino}phenyl)amino]-1-methyl-1H-pyrazolo[3,4-d]pyrimidin-4-yl}amino)benzamide;
4-[(1-Methyl-6-{[4-({[4-(morpholin-4-ylcarbonyl)phenyl]carbamoyl}amino)phenyl]amino}-1H-pyrazolo[3,4-d]pyrimidin-4-yl)amino]benzamide;
4-({[4-({4-[(4-Carbamoylphenyl)amino]-1-methyl-1H-pyrazolo[3,4-d]pyrimidin-6-yl}amino)phenyl]carbamoyl}amino)-N-(2-hydroxyethyl)-N-methylbenzamide;
4-[(6-{[4-({[2-(Dimethylamino)ethyl]carbamoyl}amino)phenyl]amino}-1-methyl-1H-pyrazolo[3,4-d]pyrimidin-4-yl)amino]benzamide;
4-({1-Methyl-6-[(4-{[(4-{[4-(propan-2-yl)piperazin-1-yl]carbonyl}phenyl)carbamoyl]amino}phenyl)amino]-1H-pyrazolo[3,4-d]pyrimidin-4-yl}amino)benzamide;
4-{[6-({4-[(Cyclopropylcarbamoyl)amino]phenyl}amino)-1-methyl-1H-pyrazolo[3,4-d]pyrimidin-4-yl]amino}benzamide;
4-{[1-Methyl-6-({4-[(propan-2-ylcarbamoyl)amino]phenyl}amino)-1H-pyrazolo[3,4-d]pyrimidin-4-yl]amino}benzamide;
N-Methyl-4-{[1-methyl-6-({4-[(pyridin-3-ylcarbamoyl)amino]phenyl}amino)-1H-pyrazolo[3,4-d]pyrimidin-4-yl]amino}benzamide;
4-({1-Methyl-6-[(4-{[(1-methylpiperidin-4-yl)carbamoyl]amino}phenyl)amino]-1H-pyrazolo[3,4-d]pyrimidin-4-yl}amino)benzamide;
4-({[4-({4-[(4-Carbamoylphenyl)amino]-1-methyl-1H-pyrazolo[3,4-d]pyrimidin-6-yl}amino)phenyl]carbamoyl}amino)-N-[2-(dimethylamino)ethyl]-N-methylbenzamide;
N,N-Dimethyl-4-{[1-methyl-6-({4-[(pyridin-3-ylcarbamoyl)amino]phenyl}amino)-1H-pyrazolo[3,4-d]pyrimidin-4-yl]amino}benzamide;
1-[4-({1-Methyl-4-[(1-oxo-2,3-dihydro-1H-isoindol-5-yl)amino]-1H-pyrazolo[3,4-d]pyrimidin-6-yl}amino)phenyl]-3-pyridin-3-ylurea; and
1-[4-({1-Methyl-4-[(2-methyl-1-oxo-2,3-dihydro-1H-isoindol-5-yl)amino]-1H-pyrazolo[3,4-d]pyrimidin-6-yl}amino)phenyl]-3-pyridin-3-ylurea; and pharmaceutically acceptable salts thereof.

17) A composition comprising a compound of claim 1 and a pharmaceutically acceptable carrier.

18) The composition of claim 17, wherein the pharmaceutically acceptable carrier suitable for oral administration and the composition comprises an oral dosage form.

19) A composition comprising a compound of claim 1; a second compound selected from the group consisting of a topoisomerase I inhibitor, a MEK ½ inhibitor, a HSP90 inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, hydroxyzine, glatiramer acetate, interferon beta-1a, interferon beta-1b, natalizumab and lavendustin A; and a pharmaceutically acceptable carrier.

20) The composition of claim 20, wherein the second compound is Avastin.

21) A method of treating a PI3K-related disorder, comprising administering to a mammal in need thereof a compound of claim 1 in an amount effective to treat a PI3K-related disorder.

22) The method of claim 21, wherein the PI3K-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

23) The method of claim 22, wherein the PI3K-related disorder is cancer.

24) The method of claim 23, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

25) A method of treating an mTOR-related disorder, comprising administering to a mammal in need thereof a compound of claim 1 in an amount effective to treat an mTOR-related disorder.

26) The method of claim 25, wherein the mTOR-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

27) The method of claim 26, wherein the mTOR-related disorder is cancer.

28) The method of claim 27, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

29) A method of treating advanced renal cell carcinoma, comprising administering to a mammal in need thereof a compound of claim 1 in an amount effective to treat advanced renal cell carcinoma.

30) A method of treating acute lymphoblastic leukemia, comprising administering to a mammal in need thereof a compound of claim 1 in an amount effective to treat acute lymphoblastic leukemia.

31) A method of treating acute malignant melanoma, comprising administering to a mammal in need thereof a compound of claim 1 in an amount effective to treat malignant melanoma.

32) A method of treating soft-tissue or bone sarcoma, comprising administering to a mammal in need thereof a compound of claim 1 in an amount effective to treat soft-tissue or bone sarcoma.

33) A method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof the composition of claim 20 in an amount effective to treat the cancer.

34) A method of inhibiting mTOR in a subject, comprising administering to a subject in need thereof a compound of claim 1 in an amount effective to inhibit mTOR.

35) A method of inhibiting PI3K in a subject, comprising administering to a subject in need thereof a compound of claim 1 in an amount effective to inhibit PI3K.

36) A method of inhibiting mTOR and PI3K together in a subject, comprising administering to a subject in need thereof a compound of claim 1 in an amount effective to inhibit mTOR and PI3K.

37) A method of synthesizing a compound of claim 2 comprising reacting a 6-chloro-1H-pyrazolo[3,4-d]pyrimidine compound of formula XV with a boronic acid of formula R²—Ar¹(B(OH)₂ 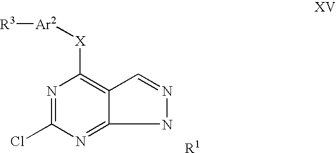
and a suitable catalyst, in which formulae Ar¹, Ar², X, and R¹-R³ are as defined above in claim 1, thereby producing a compound of formula II: 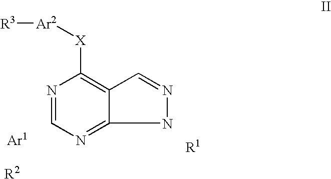
or a pharmaceutically acceptable salt thereof.

38) A method of claim 37 in which the compound of formula XV is prepared by a process comprising reacting a 4,6-dichloro-1H-pyrazolo[3,4-d]pyrimidine intermediate of formula VIII: 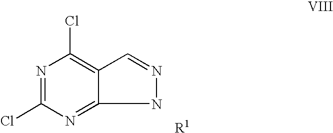
with phenols, arylmercaptans, heteroarylmercaptans, heteroarylamines, or anilines of the formula R³—Ar²—XH, thereby providing a mono chloro derivative of formula XV.
